(19) United States
(12) Patent Application Publication
(10) Pub. No.: US 2005/0042632 A1
(43) Pub. Date: Feb. 24, 2005

(54) Antibodies having specificity for nucleic acids

(75) Inventor: Susan F. Radka, Arvada, CO (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Sirna Therapeutics, Inc., Boulder, CO (US)

(21) Appl. No.: 10/820,622

(22) Filed: Apr. 8, 2004

Related U.S. Application Data
(63) Continuation-in-part of application No. 10/366,191, filed on Feb. 12, 2003.
(60) Provisional application No. 60/356,298, filed on Feb. 13, 2002.

(51) Int. Cl. C12Q 1/68; C07H 21/04; C12N 5/06; C07K 16/18

(52) U.S. Cl. 435/6; 435/69.1; 435/320.1; 435/326; 530/388.1; 536/23.53

(57) ABSTRACT

The present invention relates to antibodies, antibody conjugates, and compositions thereof, methods of antibody synthesis, and applications of antibodies useful for detecting the presence of nucleic acid molecules, including short interfering RNA (siRNA) molecules, in vivo, such as in a clinical setting. The antibodies of the invention are also useful as screening agents which allow the selection of candidate therapeutic molecules for optimum bioavailability and/or activity, and as delivery agents for cell and tissue specific delivery of nucleic acid molecules.

This application is a continuation-in-part of U.S. patent application Ser. No. 10/366,191, filed Feb. 12, 2003, which claims the benefit of U.S. Provisional Application No. 60/356,298 filed Feb. 13, 2002, both of which are hereby incorporated by reference herein in their entireties, including the drawings.

### FIELD OF THE INVENTION

The present invention relates to antibodies, antibody-conjugates, compositions, methods of synthesis, and applications thereof. One application of the invention relates to monoclonal antibodies useful for detecting the presence of target nucleic acid molecules in vivo such as in a clinical setting. The antibodies of the invention are also useful as screening agents, which allow the selection of candidate therapeutic molecules for optimum bioavailability and/or activity, and as delivery agents for cell- and tissue-specific delivery of nucleic acid molecules.

### BACKGROUND OF THE INVENTION

As therapeutic nucleic acid molecules begin to enter clinical trials, the need for methods of in vitro nucleic acid analysis and in vivo nucleic acid detection is essential. Thus, as nucleic acid therapeutics progress through preclinical studies and into clinical trials, there is a need for reagents capable of detecting such molecules in fluids (e.g., whole blood, plasma, spinal fluid and the like), cells, tissues, tissue samples and the like. For preclinical studies, addition of tags such as 2′-bromo-deoxyuridine (BrdU) and fluorescein to the nucleic acid molecule during synthesis is useful for localization purposes. In clinical trials, however, nucleic acid molecules administered therapeutically do not carry such tags. Therefore, other methods of detection and analysis need to be developed in order to assay nucleic acid-based therapeutics in a bioanalytical clinical setting.

Antibodies are highly specific and efficient analytical tools that can be used in biomedical research. Modern researchers have capitalized on this bioanalytical tool through a variety of modification techniques, including antibody engineering using recombinant DNA methods. The use of antibodies has expanded from simple diagnostic assays to the detection of molecular structures, the elucidation of gene function, the localization of gene products, and the rapid screening of biological effectors for drug discovery and testing. The use of such antibodies with fluorescent or enzymatic tags, in concert with advances in microscopy, has resulted in improved enzyme-linked immunosorbent assay (ELISA) systems. The use of ELISA based microarrays with antibodies promises to transform current paradigms of disease research and the search for new therapeutic compounds. Moreover, antibodies can serve not only as powerful research tools, but also as therapeutic compounds when conjugated with modifications such as radioisotopes and/or other chemotherapeutic compounds.

In recent years, antibodies have become well characterized through experimentation and manipulation. The typical antibody is a tetrameric molecule comprising two copies of a heavy chain (H) polypeptide which is approximately 440 amino acids long and two copies of a light-chain (L) polypeptide which is about 220 amino acids long. Each antibody-based H and L polypeptide contains a variable region and a constant region. At the terminus of each arm of the Y-shaped antibody exists a site comprising the variable termini of the H and L subunits, which together bind to a specific and unique site on an antigen, otherwise known as an epitope. Antibody technology has developed from the production and use of polyclonal antibody mixtures to the production of specific monoclonal antibodies through cell fusion techniques using, for example, mice spleen cells and cancer (myeloma) cells, to modern engineering of uniquely designed mono and divalent antibodies. Chimeric antibodies are created when the antigen-binding component of a one antibody, such as a mouse antibody, is fused to the effector component of another antibody, for example a human antibody, using genetic engineering. Monoclonal antibodies originally raised in mice, rabbits, pigs, sheep, cows, horses or the like can also be “humanized” by exchanging surface-exposed amino acids, which can be determined through molecular biological (e.g., sequencing), crystallographic and molecular modeling techniques, found on the non-human antibody to those more often found in human antibodies. Also, mice have been developed that harbor human antibody-producing elements and major histocompatibility complexes (MHCs) in place of the corresponding murine elements and complexes, such that immunization of these mice leads to the direct generation of human antibodies in the mouse. Antibodies can also be fused with a variety of other proteins that can modulate both antibody activity and localization for specific applications.

The application of short interfering RNA (siRNA) mediated RNA interference (RNAi) technology to modulation of gene expression via mRNA reduction in a research setting has been followed by the development of potential siRNA based therapeutics. As this technology proceeds through preclinical and clinical testing, there is a need for reagents and assays to detect and quantitate their presence in target organs, and other tissues. Monoclonal antibodies (mAbs) directed against epitopes on siRNA or siNA offer advantages for tissue localization over direct labeling of a strand with a fluorescent probe. With direct detection, there is no need to synthesize additional fluor-labeled siRNA or siNA for tissue localization. In addition, in vivo detection of siRNA or siNA that is destined for preclinical and clinical testing is not subject to possible changes or enhancement of tissue distribution as a result of labeling. Applicant has developed a series of mAbs directed against 2′ fluorouridine, a nucleotide modification used for in vivo stabilization of certain chemically modified siRNA and siNA constructs. These mAbs are capable of detecting siNA by immunohistochemistry in liver sections from mice dosed subcutaneously with a siNA duplex directed against Hepatitis B.

The antibodies described herein are unique and distinct from previously described antibodies. Furthermore, antibodies of the present invention are useful in a variety of applications, including but not limited to bioanalytical assays supporting clinical trials, screening candidate therapeutic molecules for optimum bioavailability and/or activity in vivo, and the in vivo delivery of certain nucleic acid molecules such as short interfering RNA (siRNA) to specific cells or tissues.

### SUMMARY OF THE INVENTION

The present invention features antibodies and antibody conjugates and compositions to facilitate the analysis of nucleic acid molecules, such as short interfering nucleic acid (siNA), in a biological system (e.g., cells, tissues, and organs) and in various organisms (e.g., humans). The antibodies provided by the instant invention also provide a useful tool for screening populations of nucleic acid molecules (e.g., siNA, siRNA, ssRNA, dsRNA, antisense, aptamers, ribozymes, enzymatic nucleic acids, DNAzymes, decoys, triplex-forming oligonucleotides, 2′,5′-oligoadenylate chimeras) to determine optimum biological activity or bioavailability. The present invention also encompasses the site-specific delivery of nucleic acid molecules (e.g., siNA, siRNA, ssRNA, dsRNA, antisense, aptamers, ribozymes, enzymatic nucleic acids, DNAzymes, decoys, triplex-forming oligonucleotides, 2′,5′-oligoadenylate chimeras) to particular cells and/or tissues. In general, the antibody-based transporters described are designed to be used either individually or as part of a multi-component system, with or without degradable linkers. The compounds of the invention described herein represent a useful bioanalytical tool for the analysis of therapeutic nucleic acid molecules (e.g., siNA, siRNA, ssRNA, dsRNA, antisense, aptamers, ribozymes, enzymatic nucleic acids, DNAzymes, decoys, triplex-forming oligonucleotides, 2′,5′-oligoadenylate chimeras) in a clinical setting, as they can be used to screen for therapeutic nucleic acid molecules in vivo, and can be used to improve delivery of certain nucleic acid molecules (e.g., siNA, siRNA, ssRNA, dsRNA, antisense, aptamers, ribozymes, enzymatic nucleic acids, DNAzymes, decoys, triplex-forming oligonucleotides, 2′,5′-oligoadenylate chimeras) to a number of cell types originating from different tissues.

In one embodiment, the invention features a monoclonal antibody (mAb) having binding affinity for nucleic acid molecules (e.g., siNA, siRNA, ssRNA, dsRNA, antisense, aptamers, ribozymes, enzymatic nucleic acids, DNAzymes, decoys, triplex-forming oligonucleotides, 2′,5′-oligoadenylate chimeras) comprising a 2′-deoxy-2′-C-allyl Uridine nucleoside and/or nucleotide. In another embodiment, the monoclonal antibody (mAb) having binding affinity for nucleic acid molecules comprising a 2′-deoxy-2′-C-allyl Uridine nucleoside and/or nucleotide is a murine IgG1ₖ antibody.

In one embodiment, the invention features a monoclonal antibody (mAb) having binding affinity for nucleic acid molecules (e.g., siNA, siRNA, ssRNA, dsRNA, antisense, aptamers, ribozymes, enzymatic nucleic acids, DNAzymes, decoys, triplex-forming oligonucleotides, 2′,5′-oligoadenylate chimeras) comprising a 2′-deoxy-2′-fluoro Uridine nucleoside and/or nucleotide. In another embodiment, the monoclonal antibody (mAb) having binding affinity for nucleic acid molecules comprising a 2′-deoxy-2′-fluoro Uridine nucleoside and/or nucleotide is a murine IgG_{2b} antibody.

In one embodiment, the invention features a monoclonal antibody (mAb) having binding affinity for nucleic acid molecules (e.g., siNA, siRNA, ssRNA, dsRNA, antisense, aptamers, ribozymes, enzymatic nucleic acids, DNAzymes, decoys, triplex-forming oligonucleotides, 2′,5′-oligoadenylate chimeras) comprising a terminal cap moiety (see for example FIG. 7). In one embodiment, the invention features a monoclonal antibody (mAb) having binding affinity for nucleic acid molecules (e.g., siNA, siRNA, ssRNA, dsRNA, antisense, aptamers, ribozymes, enzymatic nucleic acids, DNAzymes, decoys, triplex-forming oligonucleotides, 2′,5′-oligoadenylate chimeras) comprising a non-nucleotide moiety. The non-nucleotide moiety can comprise an abasic moiety, polyether, polyamine, polyamide, peptide, carbohydrate, lipid, polyhydrocarbon, or other polymeric compounds (e.g. polyethylene glycols such as those having between 2 and 100 ethylene glycol units). Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO 89/02439; Usman et al., International Publication No. WO 95/06731; Dudycz et al., International Publication No. WO 95/11910 and Ferentz and Verdine, J. Am. Chem. Soc. 1991, 113:4000, all hereby incorporated by reference herein.

In another embodiment, the invention features a monoclonal antibody (mAb) having binding affinity for nucleic acid molecules (e.g., siNA, siRNA, ssRNA, dsRNA, antisense, aptamers, ribozymes, enzymatic nucleic acids, DNAzymes, decoys, triplex-forming oligonucleotides, 2′,5′-oligoadenylate chimeras) comprising a nucleoside, nucleotide, or non-nucleotide having Formula I: 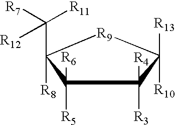
wherein each R3, R4, R5, R6, R7, R8, R10, R11 and R12 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, heterocycloalkyl, heterocycloalkaryl, aminoalkylamino, polyaklylamino, substituted silyl, or group having Formula II below; R9 is O, S, CH2, S═O, CHF, or CF2, and R13 is H or a nucleosidic base such as adenine, guanine, uracil, cytosine, thymine, 2-aminoadenosine, 5-methylcytosine, 2,6-diaminopurine, or any other non-naturally occurring base that can be employed to form a stable duplex with RNA or a non-nucleosidic base such as phenyl, naphthyl, 3-nitropyrrole, 5-nitroindole, nebularine, pyridone, pyridinone, or any other non-naturally occurring universal base that can be employed to form a stable duplex with RNA.

In another embodiment, the invention features a monoclonal antibody (mAb) having binding affinity for nucleotides and/or non-nucleotides of Formula I further comprising an internucleotide linkage having Formula II: 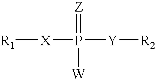
wherein each R1 and R2 is independently any nucleotide, non-nucleotide, or polynucleotide which can be naturally occurring or chemically modified, each X and Y is independently O, S, N, alkyl, or substituted alkyl, each Z and W is independently O, S, N, alkyl, substituted alkyl, O-alkyl, S-alkyl, alkaryl, or aralkyl, and wherein W, X, Y and Z are not all O.

In one embodiment, the invention features a monoclonal antibody (mAb) having binding affinity for a nucleic acid molecule comprising siNA sequences described in McSwiggen et al., U.S. Ser. Nos. 10/665,951 and 10/720,448, both incorporated by reference herein.

In one embodiment, the invention features a monoclonal antibody (mAb) having binding affinity for a nucleic acid molecule comprising SEQ ID NO: 1. In another embodiment, the invention features a monoclonal antibody (mAb) having binding affinity for a nucleic acid molecule comprising SEQ ID NO: 3. In yet another embodiment, the invention features a monoclonal antibody (mAb) having binding affinity for a nucleic acid molecule comprising SEQ ID NO: 16. In another embodiment, the invention features a monoclonal antibody (mAb) having binding affinity for a nucleic acid molecule comprising SEQ ID NO: 17.

In one embodiment, the invention features a method for generating a monoclonal antibody (mAb) having binding affinity for nucleic acid molecules comprising a 2′-deoxy-2′-C-allyl Uridine nucleoside and/or nucleotide comprising: (a) conjugating a polynucleotide comprising a 2′-deoxy-2′-C-allyl Uridine nucleoside and/or nucleotide to a carrier protein to form a polynucleotide-protein conjugate; (b) immunizing a mammal with the conjugate from (a); (c) obtaining antibody-producing cells from the immunized mammal of (b); (d) fusing the cells obtained in step (c) with myeloma cells under conditions suitable for generating a hybridoma; and (e) isolating and using the supernatant from the hybridoma of (d) in a fusion screening assay suitable for isolating the monoclonal antibody. In one embodiment, the mammal used for immunization is a mouse. In one embodiment, the 2′-deoxy-2′-C-allyl Uridine nucleotide is a 2′-deoxy-2′-C-allyl Uridine 5′-phosphate. In one embodiment, the nucleic acid molecule comprises a siNA, siRNA, ssRNA, dsRNA, antisense, aptamer, ribozyme, enzymeatic nucleic acid, DNAzyme, decoy, triplex-forming oligonucleotide, or 2′,5′-oligoadenylate chimera molecule.

In one embodiment, the invention features a method for generating a monoclonal antibody (mAb) having binding affinity for nucleic acid molecules comprising a 2′-deoxy-2′-fluoro Uridine nucleoside and/or nucleotide comprising: (a) conjugating a polynucleotide comprising a 2′-deoxy-2′-fluoro Uridine nucleoside and/or nucleotide to a carrier protein to form a polynucleotide-protein conjugate; (b) immunizing a mammal with the conjugate from (a); (c) obtaining antibody-producing cells from the immunized mammal of (b); (d) fusing the cells obtained in step (c) with myeloma cells under conditions suitable for generating a hybridoma; and (e) isolating and using the supernatant from the hybridoma of (d) in a fusion screening assay suitable for isolating the monoclonal antibody. In one embodiment, the mammal used for immunization is a mouse. In one embodiment, the 2-deoxy-2′-fluoro Uridine nucleotide is a 2′-deoxy-2′-fluoro Uridine 5′-phosphate. In one embodiment, the nucleic acid molecule comprises a siNA, siRNA, ssRNA, dsRNA, antisense, aptamer, ribozyme, enzymeatic nucleic acid, DNAzyme, decoy, triplex-forming oligonucleotide, or 2′,5′-oligoadenylate chimera molecule.

In one embodiment, the invention features a method for generating a monoclonal antibody (mAb) having binding affinity for nucleic acid molecules comprising a non-nucleotide moiety (e.g. abasic moiety) comprising: (a) conjugating a polynucleotide comprising a non-nucleotide moiety (e.g., abasic moiety) to a carrier protein to form a polynucleotide-protein conjugate; (b) immunizing a mammal with the conjugate from (a); (c) obtaining antibody-producing cells from the immunized mammal of (b); (d) fusing the cells obtained in step (c) with myeloma cells under conditions suitable for generating a hybridoma; and (e) isolating and using the supernatant from the hybridoma of (d) in a fusion screening assay suitable for isolating the monoclonal antibody. In one embodiment, the mammal used for immunization is a mouse. In one embodiment, the non-nucleotide moiety comprises a terminal cap moiety (see, for example, FIG. 7). In one embodiment, the nucleic acid molecule comprises a siNA, siRNA, ssRNA, dsRNA, antisense, aptamer, ribozyme, enzymeatic nucleic acid, DNAzyme, decoy, triplex-forming oligonucleotide, or 2′,5′-oligoadenylate chimera molecule.

In another embodiment, the invention features a method for generating a monoclonal antibody (mAb) having binding affinity for nucleic acid molecules comprising a nucleoside and/or nucleotide having Formula I comprising: (a) conjugating a polynucleotide comprising a nucleoside and/or nucleotide having Formula I to a carrier protein, to form a polynucleotide-protein conjugate; (b) immunizing a mammal with the conjugate from (a); (c) obtaining antibody-producing cells from the immunized mammal of (b); (d) fusing the cells obtained in step (c) with myeloma cells under conditions suitable for generating a hybridoma; and (e) isolating and using the supernatant from the hybridoma of (d) in a fusion screening assay suitable for isolating the monoclonal antibody. In one embodiment, the mammal used for immunization is a mouse. In one embodiment, the nucleotide having Formula I is a nucleotide comprising a 5′-phosphate or biotin moiety. In one embodiment, the nucleic acid molecule comprises a siNA, siRNA, ssRNA, dsRNA, antisense, aptamer, ribozyme, enzymeatic nucleic acid, DNAzyme, decoy, triplex-forming oligonucleotide, or 2′,5′-oligoadenylate chimera molecule.

In another embodiment, the invention features a method for generating a monoclonal antibody (mAb) having binding affinity for a nucleic acid molecule having SEQ ID NO: 1, 3, 16, or 17 comprising: (a) conjugating a nucleic acid molecule having SEQ ID NO: 1, 3, 16, or 17 to a carrier protein, to form a nucleic acid-protein conjugate; (b) immunizing a mammal with the conjugate from (a); (c) obtaining antibody-producing cells from the immunized mammal of (b); (d) fusing the cells obtained in step (c) with myeloma cells under conditions suitable for generating a hybridoma; and (e) isolating and using supernatant from the hybridoma of (d) in a fusion screening assay suitable for isolating the monoclonal antibody. In one embodiment, the mammal used for immunization is a mouse.

Examples of suitable carrier proteins include, but are not limited to, bovine serum albumin (BSA) and keyhole limpet hemocyanin (KLH).

Monoclonal antibodies of the invention can be used to detect the presence of one or more target nucleic acid molecules in a biological system, including, but not limited to, a tissue, a cell or a cell lysate. Detection of target nucleic acid molecules can be used, for example, to determine bioavailabilty of said target molecule. Examples of target nucleic acid molecules include, but are not limited to, nucleic acid molecules having SEQ ID NOS: 1, 3, 16, or 17 and/or having at least one 2′-deoxy-2′-C-allyl Uridine nucleotide. In another embodiment, the target nucleic acid molecule comprises a siNA, siRNA, ssRNA, dsRNA, antisense, aptamer, ribozyme, enzymeatic nucleic acid, DNAzyme, decoy, triplex-forming oligonucleotide, or 2′,5′-oligoadenylate chimera molecule.

The invention also features mAb conjugates. The monoclonal antibodies of the present invention can be conjugated to a predetermined compound or molecule that is capable of interacting with the target nucleic acid molecule in the system and providing a detectable signal or response. Compounds and molecules known in the art that can be used in these applications include, but are not limited to, antibodies, labeled antibodies, allozymes, aptamers, labeled nucleic acid probes, molecular beacons, fluorescent molecules, radioisotopes, polysaccharides, and other compounds capable of interacting with the target nucleic acid molecule and generating a detectable signal upon target interaction, and the like. Examples of such compounds are further described in U.S. Ser. No. 09/800,594, filed on Mar. 6, 2001, which is incorporated herein by reference in its entirety.

In another embodiment, polyethylene glycol (PEG) can be covalently attached to the mAb compounds of the present invention. The attached PEG can be any molecular weight, for example, from about 2000 to about 50,000 daltons (Da).

In one embodiment, the invention features a method of detecting the presence of a nucleic acid molecule having a 2′-deoxy-2′-C-allyl Uridine nucleotide in a patient or subject, comprising: (a) obtaining a biological sample from the patient or subject; and (b) contacting the biological sample of (a) with a monoclonal antibody or monoclonal antibody conjugate of the invention under conditions suitable for detecting the presence of the nucleic acid molecule in the patient or subject. In one embodiment, the nucleic acid molecule is a siNA molecule. In another embodiment, the nucleic acid molecule comprises a siRNA, ssRNA, dsRNA, antisense, aptamer, ribozyme, enzymeatic nucleic acid, DNAzyme, decoy, triplex-forming oligonucleotide, or 2′,5′-oligoadenylate chimera molecule.

In one embodiment, the invention features a method of detecting the presence of a nucleic acid molecule having a 2′-deoxy-2′-fluoro Uridine nucleotide in a patient or subject comprising: (a) obtaining a biological sample from the patient or subject; and (b) contacting the biological sample of (a) with a monoclonal antibody or monoclonal antibody conjugate of the invention under conditions suitable for detecting the presence of the nucleic acid molecule in the patient or subject. In one embodiment, the nucleic acid molecule is a siNA molecule. In another embodiment, the nucleic acid molecule comprises a siRNA, ssRNA, dsRNA, antisense, aptamer, ribozyme, enzymeatic nucleic acid, DNAzyme, decoy, triplex-forming oligonucleotide, or 2′,5′-oligoadenylate chimera molecule.

In one embodiment, the invention features a method of detecting the presence of a nucleic acid molecule comprising a non-nucleotide moiety in a patient or subject comprising: (a) obtaining a biological sample from the patient or subject; and (b) contacting the biological sample of (a) with a monoclonal antibody or monoclonal antibody conjugate of the invention under conditions suitable for detecting the presence of the nucleic acid molecule in the patient or subject. In one embodiment, the nucleic acid molecule is a siNA molecule. In another embodiment, the nucleic acid molecule comprises a siRNA, ssRNA, dsRNA, antisense, aptamer, ribozyme, enzymeatic nucleic acid, DNAzyme, decoy, triplex-forming oligonucleotide, or 2′,5′-oligoadenylate chimera molecule.

In another embodiment, the invention features a method of detecting the presence of a nucleic acid molecule comprising a nucleoside and/or nucleotide having Formula I in a patient or subject comprising: (a) obtaining a biological sample from the patient or subject; and (b) contacting the biological sample of (a) with a monoclonal antibody or monoclonal antibody conjugate of the invention under conditions suitable for detecting the presence of the nucleic acid molecule in the patient or subject. In one embodiment, the nucleic acid molecule is a siNA molecule. In another embodiment, the nucleic acid molecule comprises a siRNA, ssRNA, dsRNA, antisense, aptamer, ribozyme, enzymeatic nucleic acid, DNAzyme, decoy, triplex-forming oligonucleotide, or 2′,5′-oligoadenylate chimera molecule.

In another embodiment, the invention features a method of detecting the presence of a nucleic acid molecule having SEQ ID NO: 1, 3, 16, or 17 in a patient or subject comprising: (a) obtaining a biological sample from the patient or subject; and (b) contacting the biological sample of (a) with a monoclonal antibody or monoclonal antibody conjugate of the invention under conditions suitable for detecting the presence of the nucleic acid molecule in the patient or subject.

In one embodiment, the invention features a method for determining the level of a nucleic acid molecule in a mammal comprising: (a) administering the candidate nucleic acid molecule to the mammal; (b) obtaining a biological sample from the mammal; (c) contacting the biological sample of (b) with a monoclonal antibody or monoclonal antibody conjugate of the invention under conditions suitable for detecting the presence of the nucleic acid molecule in the sample, and (d) assaying for the nucleic acid molecule in the sample under conditions suitable to determine the level of the nucleic acid molecule in the biological sample and/or mammal. In one embodiment, the mammal is non-human (e.g., including, but not limited to, a mouse, rat, rabbit, or pig). In another embodiment, the mammal is a human. In another embodiment, the nucleic acid molecule comprises a siNA, siRNA, ssRNA, dsRNA, antisense, aptamer, ribozyme, enzymeatic nucleic acid, DNAzyme, decoy, triplex-forming oligonucleotide, or 2′,5′-oligoadenylate chimera molecule.

Examples of biological samples include, but are not limited to, samples derived from cells and/or tissues, such as serum, blood, urine, and cell lysates.

The invention also features monoclonal antibodies and monoclonal antibody conjugates that direct the in vivo localization of target nucleic acid molecules. In one embodiment, the invention features a mAb that directs the in vivo localization of a target nucleic acid molecule. In another embodiment, the invention features a mAb conjugate that directs the in vivo localization of a target nucleic acid molecule. In one embodiment, the mAb conjugate comprises a mAb of the invention and one or more conjugated groups or moieties capable of directing the in vivo localization of a target nucleic acid molecule. Such conjugated groups or moieties can include proteins, peptides, polypeptides, receptor ligands, lipids, phospholipids, carbohydrates, polycations, polyethylene glycols, or other polymers or molecules, such as biologically active molecules that facilitate bioavailability or enable cell or tissue specific localization of pendant molecules described above. Interaction of the conjugated mAb with a nucleic acid molecule is expected to facilitate pharmacokinetics and the in vivo bioavailability of a nucleic acid molecule to a particular cell and/or tissue type of an organism, such as a patient or subject. In one embodiment, the target nucleic acid molecule comprises a siNA, siRNA, ssRNA, dsRNA, antisense, aptamer, ribozyme, enzymeatic nucleic acid, DNAzyme, decoy, triplex-forming oligonucleotide, or 2′,5′-oligoadenylate chimera molecule.

Another embodiment of the invention encompasses mAb conjugates comprising targeting components that increase the transport of other impermeable and/or lipophilic compounds into cells. Targeting components can include ligands for cell surface receptors including, but not limited to, peptides and proteins, glycolipids, lipids, carbohydrates, and their synthetic variants, (e.g., including, but not limited to, ligands for asialoglycoprotein (ASGPr) receptors or folate receptors). Methods for generating conjugated antibodies are known in the art and are described in, for example, Thorpe et al., U.S. Pat. No. 6,342,221, incorporated by reference herein.

The compounds or conjugates and methods of the present invention are useful for introducing nucleotides, nucleosides, nucleic acid molecules, lipids, peptides, proteins, and/or non-nucleosidic small molecules into a cell. For example, the invention can be used for nucleotide, nucleoside, nucleic acid, lipids, peptides, proteins, and/or non-nucleosidic small molecule delivery where the corresponding target site of action exists intracellularly.

In one specific embodiment, the compounds of the present invention are conjugates of molecules that interact with ASGPr, RGD peptide, or folate receptors, and also provide a number of features that allow the efficient delivery and subsequent release of conjugated compounds across biological membranes. The compounds can utilize chemical linkages between galactose, galactosamine, or N-acetyl galactosamine substituents and the compound to be delivered which are of such length that they interact preferentially with ASGPr receptors. In one embodiment, the chemical linkages between RGD, folate, galactose, galactosamine, or N-acetyl galactosamine substituents and the compound to be delivered can be designed as degradable linkages, for example, by utilizing a phosphate linkage that is proximal to a nucleophile, such as a hydroxyl group, or utilizing a nucleic acid linker comprising ribonucleotides. Deprotonation of the hydroxyl group or an equivalent group, as a result of pH or interaction with a nuclease, can result in nucleophilic attack of the phosphate resulting in a cyclic phosphate intermediate that can then be hydrolyzed. This cleavage mechanism is analogous to RNA cleavage in the presence of a base or RNA nuclease. Alternately, other degradable linkages can be selected that respond to various factors, for example, UV irradiation (photolabile linker), cellular nucleases, pH, or temperature. The use of degradable linkages allows the delivered compound to be released in a predetermined system, for example, in the cytoplasm of a cell, or in a particular cellular organelle. Non-limiting examples of such linkers are described in Vargeese et al., U.S. Ser. No. 10/201,394, incorporated by reference herein.

In one embodiment, polyethylene glycol (PEG) can be covalently attached to mAb compounds of the present invention. The attached PEG can be any molecular weight, for example, from about 2,000 to about 50,000 daltons (Da).

The monoclonal antibodies of the invention can be conjugated directly to the described compounds to form a mAb conjugate or can be conjugated via a linker, such as a linker described herein.

In one embodiment, a patient or subject contemplated by the invention is a patient or subject treated with a therapeutic nucleic acid molecule having a 2′-deoxy-2′-C-allyl Uridine, 2′-deoxy-2′-fluoro Uridine nucleotide, or non-nucleotide moiety. Such a therapeutic nucleic acid molecule can include, but is not limited to, an enzymatic nucleic acid molecule or ribozyme, allozyme, antisense nucleic acid, 2-5A antisense chimera, triplex forming oligonucleotide, decoy RNA, dsRNA, siRNA, siNA, aptamer, and/or antisense nucleic acid comprising nucleic acid-cleaving chemical groups, which comprise a 2′-deoxy-2′-C-allyl Uridine, 2′-deoxy-2′-fluoro Uridine nucleotide, or non-nucleotide moiety.

In one embodiment, a patient or subject contemplated by the invention is a patient or subject treated with a therapeutic nucleic acid molecule comprising a nucleoside and/or nucleotide having Formula I. Such a therapeutic nucleic acid molecule can include, but is not limited to, an enzymatic nucleic acid molecule, allozyme, antisense nucleic acid, 2-5A antisense chimera, triplex forming oligonucleotide, decoy RNA, dsRNA, siRNA, siNA, aptamer, and/or antisense nucleic acid comprising nucleic acid-cleaving chemical groups, which comprise a nucleoside and/or nucleotide having Formula I.

In one embodiment, a patient or subject contemplated by the invention is a patient or subject treated with a therapeutic nucleic acid molecule comprising SEQ ID NO: 1, 3, 16, or 17.

In one embodiment, the invention features a method for screening candidate nucleic acid molecules for bioavailability in a mammal comprising: (a) administering the candidate nucleic acid molecule to the mammal; (b) obtaining a biological sample from the mammal, including, but not limited to, a blood sample or tissue sample; and (c) contacting the biological sample of (b) with a monoclonal antibody or monoclonal antibody conjugate of the invention under conditions suitable for detecting the presence of the nucleic acid molecule in the sample. In one embodiment, the mammal is non-human (e.g., including, but not limited to, a mouse, rat, rabbit, or pig). In another embodiment, the mammal is a human. In one embodiment, the candidate nucleic acid molecule is a siNA molecule.

The methods of the present invention can be used to determine the effect that various modifications to the structure of a nucleic acid molecule can have on bioavailability of the nucleic acid molecule. Such modifications include, but are not limited to, complete or partial chemical modification of a nucleic acid backbone, sugar, base, or any combination thereof, and/or conjugation of the nucleic acid molecule with various substituent groups or biologically active molecules that modulate the distribution, pharmacokinetics, pharmacodynamics, and/or bioavailability of the nucleic acid molecule. Non-limiting examples of such chemical modifications and conjugates are described in McSwiggen et al., WO 03/70918 and Vargeese et al., U.S. Ser. No. 10/201,394.

The compounds of the invention, including, but not limited to, monoclonal antibodies and conjugates thereof, can be administered directly to a patient or subject, can be added directly to cells or tissues isolated from a patient or subject, or can be complexed with cationic lipids, packaged within liposomes, or otherwise delivered to target cells or tissues. In one embodiment, the invention features a composition comprising a monoclonal antibody compound, i.e., monoclonal antibody or monoclonal antibody conjugate, and a pharmaceutically acceptable carrier, for example, any of the carriers described for the compositions and formulations discussed herein.

The mAb compound(s) and compositions thereof can be locally administered to relevant tissues ex vivo, or in vivo through injection or infusion pump, with or without their incorporation into biopolymers. The mAb compounds and compositions of the instant invention, individually, or in combination or conjunction with other drugs, can be used to treat various diseases or conditions such as cancer (including, but not limited to, breast, lung, prostate, colorectal, brain, esophageal, bladder, pancreatic, cervical, head and neck, and ovarian cancer, melanoma, lymphoma, glioma, and multidrug resistant cancers) or viral infections (including, but not limited to HIV, HBV, HCV, CMV, RSV, HSV, poliovirus, influenza, rhinovirus, west Nile virus, Ebola virus, foot and mouth virus, and papilloma virus infection). For example, to treat a disease or condition associated with the levels of a pathogenic protein, the patient or a patient's biological sample can be treated, as is evident to those skilled in the art, individually or in combination with one or more drugs under conditions suitable for the treatment.

In a further embodiment, the mAb, mAb conjugates and compositions thereof can be used in combination with other known treatments to treat conditions or diseases discussed above. For example, the mAb compounds and compositions of the present invention can be used in combination with one or more known therapeutic agents to treat breast, lung, prostate, colorectal, brain, esophageal, bladder, pancreatic, cervical, head and neck, and/or ovarian cancer, melanoma, lymphoma, glioma, multidrug resistant cancers, and/or HIV, HBV, HCV, CMV, RSV, HSV, poliovirus, influenza, rhinovirus, west Nile virus, Ebola virus, foot and mouth virus, and papilloma virus infection. The foregoing list is provided for illustrative purposes only and is not to be construed as limiting the invention in any way.

### BRIEF DESCRIPTION OF THE DRAWINGS

FIG. 1 shows a non-limiting example of a scheme for the synthesis of siNA molecules. The complementary siNA sequence strands, strand 1 and strand 2, are synthesized in tandem and are connected by a cleavable linkage, such as a nucleotide succinate or abasic succinate, which can be the same or different from the cleavable linker used for solid phase synthesis on a solid support. The synthesis can be either solid phase or solution phase, in the example shown, the synthesis is a solid phase synthesis. The synthesis is performed such that a protecting group, such as a dimethoxytrityl group, remains intact on the terminal nucleotide of the tandem oligonucleotide. Upon cleavage and deprotection of the oligonucleotide, the two siNA strands spontaneously hybridize to form a siNA duplex, which allows the purification of the duplex by utilizing the properties of the terminal protecting group, for example by applying a trityl on purification method wherein only duplexes/oligonucleotides with the terminal protecting group are isolated.

FIG. 2 shows a MALDI-TOF mass spectrum of a purified siNA duplex synthesized by a method of the invention. The two peaks shown correspond to the predicted mass of the separate siNA sequence strands. This result demonstrates that the siNA duplex generated from tandem synthesis can be purified as a single entity using a simple trityl-on purification methodology.

FIG. 3 shows a non-limiting proposed mechanistic representation of target RNA degradation involved in RNAi. Double-stranded RNA (dsRNA), which is generated by RNA-dependent RNA polymerase (RdRP) from foreign single-stranded RNA, for example viral, transposon, or other exogenous RNA, activates the DICER enzyme that in turn generates siNA duplexes. Alternately, synthetic or expressed siNA can be introduced directly into a cell by appropriate means. An active siNA complex forms which recognizes a target RNA, resulting in degradation of the target RNA by the RISC endonuclease complex or in the synthesis of additional RNA by RNA-dependent RNA polymerase (RdRP), which can activate DICER and result in additional siNA molecules, thereby amplifying the RNAi response.

FIG. 4A-F shows non-limiting examples of chemically-modified siNA constructs of the present invention. In the figure, N stands for any nucleotide (adenosine, guanosine, cytosine, uridine, or optionally thymidine, for example thymidine can be substituted in the overhanging regions designated by parenthesis (N N). Various modifications are shown for the sense and antisense strands of the siNA constructs.

FIG. 4A: The sense strand comprises 21 nucleotides wherein the two terminal 3′-nucleotides are optionally base paired and wherein all nucleotides present are ribonucleotides except for (N N) nucleotides, which can comprise ribonucleotides, deoxynucleotides, universal bases, or other chemical modifications described herein. The antisense strand comprises 21 nucleotides, optionally having a 3′-terminal glyceryl moiety wherein the two terminal 3′-nucleotides are optionally complementary to the target RNA sequence, and wherein all nucleotides present are ribonucleotides except for (N N) nucleotides, which can comprise ribonucleotides, deoxynucleotides, universal bases, or other chemical modifications described herein. A modified internucleotide linkage, such as a phosphorothioate, phosphorodithioate or other modified internucleotide linkage as described herein, shown as “s”, optionally connects the (N N) nucleotides in the antisense strand.

FIG. 4B: The sense strand comprises 21 nucleotides wherein the two terminal 3′-nucleotides are optionally base paired and wherein all pyrimidine nucleotides that may be present are 2′deoxy-2′-fluoro modified nucleotides and all purine nucleotides that may be present are 2′-O-methyl modified nucleotides except for (N N) nucleotides, which can comprise ribonucleotides, deoxynucleotides, universal bases, or other chemical modifications described herein. The antisense strand comprises 21 nucleotides, optionally having a 3′-terminal glyceryl moiety and wherein the two terminal 3′-nucleotides are optionally complementary to the target RNA sequence, and wherein all pyrimidine nucleotides that may be present are 2′-deoxy-2′-fluoro modified nucleotides and all purine nucleotides that may be present are 2′-O-methyl modified nucleotides except for (N N) nucleotides, which can comprise ribonucleotides, deoxynucleotides, universal bases, or other chemical modifications described herein. A modified internucleotide linkage, such as a phosphorothioate, phosphorodithioate or other modified internucleotide linkage as described herein, shown as “s”, optionally connects the (N N) nucleotides in the sense and antisense strand.

FIG. 4C: The sense strand comprises 21 nucleotides having 5′- and 3′-terminal cap moieties wherein the two terminal 3′-nucleotides are optionally base paired and wherein all pyrimidine nucleotides that may be present are 2′-O-methyl or 2′-deoxy-2′-fluoro modified nucleotides except for (N N) nucleotides, which can comprise ribonucleotides, deoxynucleotides, universal bases, or other chemical modifications described herein. The antisense strand comprises 21 nucleotides, optionally having a 3′-terminal glyceryl moiety and wherein the two terminal 3′-nucleotides are optionally complementary to the target RNA sequence, and wherein all pyrimidine nucleotides that may be present are 2′-deoxy-2′-fluoro modified nucleotides except for (N N) nucleotides, which can comprise ribonucleotides, deoxynucleotides, universal bases, or other chemical modifications described herein. A modified internucleotide linkage, such as a phosphorothioate, phosphorodithioate or other modified internucleotide linkage as described herein, shown as “s”, optionally connects the (N N) nucleotides in the antisense strand.

FIG. 4D: The sense strand comprises 21 nucleotides having 5′- and 3′-terminal cap moieties wherein the two terminal 3′-nucleotides are optionally base paired and wherein all pyrimidine nucleotides that may be present are 2′-deoxy-2′-fluoro modified nucleotides except for (N N) nucleotides, which can comprise ribonucleotides, deoxynucleotides, universal bases, or other chemical modifications described herein and wherein and all purine nucleotides that may be present are 2′-deoxy nucleotides. The antisense strand comprises 21 nucleotides, optionally having a 3′-terminal glyceryl moiety and wherein the two terminal 3′-nucleotides are optionally complementary to the target RNA sequence, wherein all pyrimidine nucleotides that may be present are 2′-deoxy-2′-fluoro modified nucleotides and all purine nucleotides that may be present are 2′-O-methyl modified nucleotides except for (N N) nucleotides, which can comprise ribonucleotides, deoxynucleotides, universal bases, or other chemical modifications described herein. A modified internucleotide linkage, such as a phosphorothioate, phosphorodithioate or other modified internucleotide linkage as described herein, shown as “s”, optionally connects the (N N) nucleotides in the antisense strand.

FIG. 4E: The sense strand comprises 21 nucleotides having 5′- and 3′-terminal cap moieties wherein the two terminal 3′-nucleotides are optionally base paired and wherein all pyrimidine nucleotides that may be present are 2′-deoxy-2′-fluoro modified nucleotides except for (N N) nucleotides, which can comprise ribonucleotides, deoxynucleotides, universal bases, or other chemical modifications described herein. The antisense strand comprises 21 nucleotides, optionally having a 3′-terminal glyceryl moiety and wherein the two terminal 3′-nucleotides are optionally complementary to the target RNA sequence, and wherein all pyrimidine nucleotides that may be present are 2′-deoxy-2′-fluoro modified nucleotides and all purine nucleotides that may be present are 2′-O-methyl modified nucleotides except for (N N) nucleotides, which can comprise ribonucleotides, deoxynucleotides, universal bases, or other chemical modifications described herein. A modified internucleotide linkage, such as a phosphorothioate, phosphorodithioate or other modified internucleotide linkage as described herein, shown as “s”, optionally connects the (N N) nucleotides in the antisense strand.

FIG. 4F: The sense strand comprises 21 nucleotides having 5′- and 3′-terminal cap moieties wherein the two terminal 3′-nucleotides are optionally base paired and wherein all pyrimidine nucleotides that may be present are 2′-deoxy-2′-fluoro modified nucleotides except for (N N) nucleotides, which can comprise ribonucleotides, deoxynucleotides, universal bases, or other chemical modifications described herein and wherein and all purine nucleotides that may be present are 2′-deoxy nucleotides. The antisense strand comprises 21 nucleotides, optionally having a 3′-terminal glyceryl moiety and wherein the two terminal 3′-nucleotides are optionally complementary to the target RNA sequence, and having one 3′-terminal phosphorothioate internucleotide linkage and wherein all pyrimidine nucleotides that may be present are 2′-deoxy-2′-fluoro modified nucleotides and all purine nucleotides that may be present are 2′-deoxy nucleotides except for (N N) nucleotides, which can comprise ribonucleotides, deoxynucleotides, universal bases, or other chemical modifications described herein. A modified internucleotide linkage, such as a phosphorothioate, phosphorodithioate or other modified internucleotide linkage as described herein, shown as “s”, optionally connects the (N N) nucleotides in the antisense strand. The antisense strand of constructs A-F comprise sequence complementary to any target nucleic acid sequence of the invention. Furthermore, when a glyceryl moiety (L) is present at the 3′-end of the antisense strand for any construct shown in FIG. 4A-F, the modified internucleotide linkage is optional.

FIG. 5A-F shows non-limiting examples of specific chemically-modified siNA sequences of the invention. A-F applies the chemical modifications described in FIG. 4A-F to an VEGFR1 siNA sequence. Such chemical modifications can be applied to any known sequence.

FIG. 6 shows non-limiting examples of different siNA constructs of the invention. The examples shown (constructs 1, 2, and 3) have 19 representative base pairs; however, different embodiments of the invention include any number of base pairs described herein. Bracketed regions represent nucleotide overhangs, for example comprising about 1, 2, 3, or 4 nucleotides in length, preferably about 2 nucleotides. Constructs 1 and 2 can be used independently for RNAi activity. Construct 2 can comprise a polynucleotide or non-nucleotide linker, which can optionally be designed as a biodegradable linker. In one embodiment, the loop structure shown in construct 2 can comprise a biodegradable linker that results in the formation of construct 1 in vivo and/or in vitro. In another example, construct 3 can be used to generate construct 2 under the same principle wherein a linker is used to generate the active siNA construct 2 in vivo and/or in vitro, which can optionally utilize another biodegradable linker to generate the active siNA construct 1 in vivo and/or in vitro. As such, the stability and/or activity of the siNA constructs can be modulated based on the design of the siNA construct for use in vivo or in vitro and/or in vitro.

FIG. 7 shows non-limiting examples of different stabilization chemistries (1-10) that can be used, for example, to stabilize the 3′-end of siNA sequences of the invention, including (1) [3-3′]-inverted deoxyribose; (2) deoxyribonucleotide; (3) [5′-3′]-3′-deoxyribonucleotide; (4) [5′-3′]-ribonucleotide; (5) [5′-3′]-3′-O-methyl ribonucleotide; (6) 3′-glyceryl; (7) [3′-5′]-3′-deoxyribonucleotide; (8) [3′-3′]-deoxyribonucleotide; (9) [5′-2′]-deoxyribonucleotide; and (10) [5-3′]-dideoxyribonucleotide. In addition to modified and unmodified backbone chemistries indicated in the figure, these chemistries can be combined with different backbone modifications as described herein, for example, backbone modifications having Formula I. In addition, the 2′-deoxy nucleotide shown 5′ to the terminal modifications shown can be another modified or unmodified nucleotide or non-nucleotide described herein, for example modifications having any of Formulae I-VII or any combination thereof.

FIG. 8 shows non-limiting examples of phosphorylated siNA molecules of the invention, including linear and duplex constructs and asymmetric derivatives thereof.

FIG. 9 shows non-limiting examples of chemically modified terminal phosphate groups of the invention.

FIG. 10A shows a non-limiting example of methodology used to design self complementary duplex forming oligonucleotide (DFO) constructs utilizing palidrome and/or repeat nucleic acid sequences that are identifed in a target nucleic acid sequence. (i) A palindrome or repeat sequence is identified in a nucleic acid target sequence. (ii) A sequence is designed that is complementary to the target nucleic acid sequence and the palindrome sequence. (iii) An inverse repeat sequence of the non-palindrome/repeat portion of the complementary sequence is appended to the 3′-end of the complementary sequence to generate a self complimentary DFO molecule comprising sequence complementary to the nucleic acid target. (iv) The DFO molecule can self-assemble to form a double stranded oligonucleotide. FIG. 10B shows a non-limiting representative example of a duplex forming oligonucleotide sequence. FIG. 10C shows a non-limiting example of the self assembly schematic of a representative duplex forming oligonucleotide sequence. FIG. 10D shows a non-limiting example of the self assembly schematic of a representative duplex forming oligonucleotide sequence followed by interaction with a target nucleic acid sequence resulting in modulation of gene expression.

FIG. 11 shows a non-limiting example of the design of self complementary DFO constructs utilizing palidrome and/or repeat nucleic acid sequences that are incorporated into the DFO constructs that have sequence complementary to any target nucleic acid sequence of interest. Incorporation of these palindrome/repeat sequences allow the design of DFO constructs that form duplexes in which each strand is capable of mediating modulation of target gene expression, for example by RNAi. First, the target sequence is identified. A complementary sequence is then generated in which nucleotide or non-nucleotide modifications (shown as X or Y) are introduced into the complementary sequence that generate an artificial palindrome (shown as XYXYXY in the Figure). An inverse repeat of the non-palindrome/repeat complementary sequence is appended to the 3′-end of the complementary sequence to generate a self complmentary DFO comprising sequence complementary to the nucleic acid target. The DFO can self-assemble to form a double stranded oligonucleotide.

FIG. 12 shows non-limiting examples of multifunctional siNA molecules of the invention comprising two separate polynucleotide sequences that are each capable of mediating RNAi directed cleavage of differing target nucleic acid sequences. FIG. 12A shows a non-limiting example of a multifunctional siNA molecule having a first region that is complementary to a first target nucleic acid sequence (complementary region 1) and a second region that is complementary to a second target nucleic acid sequence (complementary region 2), wherein the first and second complementary regions are situated at the 3′-ends of each polynucleotide sequence in the multifunctional siNA. The dashed portions of each polynucleotide sequence of the multifunctional siNA construct have complementarity with regard to corresponding portions of the siNA duplex, but do not have complementarity to the target nucleic acid sequences. FIG. 12B shows a non-limiting example of a multifunctional siNA molecule having a first region that is complementary to a first target nucleic acid sequence (complementary region 1) and a second region that is complementary to a second target nucleic acid sequence (complementary region 2), wherein the first and second complementary regions are situated at the 5′-ends of each polynucleotide sequence in the multifunctional siNA. The dashed portions of each polynucleotide sequence of the multifunctional siNA construct have complementarity with regard to corresponding portions of the siNA duplex, but do not have complementarity to the target nucleic acid sequences.

FIG. 13 shows non-limiting examples of multifunctional siNA molecules of the invention comprising a single polynucleotide sequence comprising distinct regions that are each capable of mediating RNAi directed cleavage of differing target nucleic acid sequences. FIG. 13A shows a non-limiting example of a multifunctional siNA molecule having a first region that is complementary to a first target nucleic acid sequence (complementary region 1) and a second region that is complementary to a second target nucleic acid sequence (complementary region 2), wherein the second complementary region is situated at the 3′-end of the polynucleotide sequence in the multifunctional siNA. The dashed portions of each polynucleotide sequence of the multifunctional siNA construct have complementarity with regard to corresponding portions of the siNA duplex, but do not have complementarity to the target nucleic acid sequences. FIG. 13B shows a non-limiting example of a multifunctional siNA molecule having a first region that is complementary to a first target nucleic acid sequence (complementary region 1) and a second region that is complementary to a second target nucleic acid sequence (complementary region 2), wherein the first complementary region is situated at the 5′-end of the polynucleotide sequence in the multifunctional siNA. The dashed portions of each polynucleotide sequence of the multifunctional siNA construct have complementarity with regard to corresponding portions of the siNA duplex, but do not have complementarity to the target nucleic acid sequences. In one embodiment, these multifunctional siNA constructs are processed in vivo or in vitro to generate multifunctional siNA constructs as shown in FIG. 12.

FIG. 14 shows non-limiting examples of multifunctional siNA molecules of the invention comprising two separate polynucleotide sequences that are each capable of mediating RNAi directed cleavage of differing target nucleic acid sequences and wherein the multifunctional siNA construct further comprises a self complementary, palindrome, or repeat region, thus enabling shorter bifunctional siNA constructs that can mediate RNA interference against differing target nucleic acid sequences. FIG. 14A shows a non-limiting example of a multifunctional siNA molecule having a first region that is complementary to a first target nucleic acid sequence (complementary region 1) and a second region that is complementary to a second target nucleic acid sequence (complementary region 2), wherein the first and second complementary regions are situated at the 3′-ends of each polynucleotide sequence in the multifunctional siNA, and wherein the first and second complementary regions further comprise a self complementary, palindrome, or repeat region. The dashed portions of each polynucleotide sequence of the multifunctional siNA construct have complementarity with regard to corresponding portions of the siNA duplex, but do not have complementarity to the target nucleic acid sequences. FIG. 14B shows a non-limiting example of a multifunctional siNA molecule having a first region that is complementary to a first target nucleic acid sequence (complementary region 1) and a second region that is complementary to a second target nucleic acid sequence (complementary region 2), wherein the first and second complementary regions are situated at the 5′-ends of each polynucleotide sequence in the multifunctional siNA, and wherein the first and second complementary regions further comprise a self complementary, palindrome, or repeat region. The dashed portions of each polynucleotide sequence of the multifunctional siNA construct have complementarity with regard to corresponding portions of the siNA duplex, but do not have complementarity to the target nucleic acid sequences.

FIG. 15 shows non-limiting examples of multifunctional siNA molecules of the invention comprising a single polynucleotide sequence comprising distinct regions that are each capable of mediating RNAi directed cleavage of differing target nucleic acid sequences and wherein the multifunctional siNA construct further comprises a self complementary, palindrome, or repeat region, thus enabling shorter bifunctional siNA constructs that can mediate RNA interference against differing target nucleic acid sequences. FIG. 15A shows a non-limiting example of a multifunctional siNA molecule having a first region that is complementary to a first target nucleic acid sequence (complementary region 1) and a second region that is complementary to a second target nucleic acid sequence (complementary region 2), wherein the second complementary region is situated at the 3′-end of the polynucleotide sequence in the multifunctional siNA, and wherein the first and second complementary regions further comprise a self complementary, palindrome, or repeat region. The dashed portions of each polynucleotide sequence of the multifunctional siNA construct have complementarity with regard to corresponding portions of the siNA duplex, but do not have complementarity to the target nucleic acid sequences. FIG. 15B shows a non-limiting example of a multifunctional siNA molecule having a first region that is complementary to a first target nucleic acid sequence (complementary region 1) and a second region that is complementary to a second target nucleic acid sequence (complementary region 2), wherein the first complementary region is situated at the 5′-end of the polynucleotide sequence in the multifunctional siNA, and wherein the first and second complementary regions further comprise a self complementary, palindrome, or repeat region. The dashed portions of each polynucleotide sequence of the multifunctional siNA construct have complementarity with regard to corresponding portions of the siNA duplex, but do not have complementarity to the target nucleic acid sequences. In one embodiment, these multifunctional siNA constructs are processed in vivo or in vitro to generate multifunctional siNA constructs as shown in FIG. 14.

FIG. 16 shows a non-limiting example of how multifunctional siNA molecules of the invention can target two separate target nucleic acid molecules, such as separate RNA molecules encoding differing proteins, for example a cytokine and its corresponding receptor, differing viral strains, a virus and a cellular protein involved in viral infection or replication, or differing proteins involved in a common or divergent biologic pathway that is implicated in the maintenance of progression of disease. Each strand of the multifunctional siNA construct comprises a region having complementarity to separate target nucleic acid molecules. The multifunctional siNA molecule is designed such that each strand of the siNA can be utilized by the RISC complex to initiate RNA interferance mediated cleavage of its corresponding target. These design parameters can include destabilization of each end of the siNA construct (see for example Schwarz et al., 2003, Cell, 115, 199-208). Such destabilization can be accomplished for example by using guanosine-cytidine base pairs, alternate base pairs (e.g., wobbles), or destabilizing chemically modified nucleotides at terminal nucleotide positions as is known in the art.

FIG. 17 shows a non-limiting example of how multifunctional siNA molecules of the invention can target two separate target nucleic acid sequences within the same target nucleic acid molecule, such as alternate coding regions of a RNA, coding and non-coding regions of a RNA, or alternate splice variant regions of a RNA. Each strand of the multifunctional siNA construct comprises a region having complementarity to the separate regions of the target nucleic acid molecule. The multifunctional siNA molecule is designed such that each strand of the siNA can be utilized by the RISC complex to initiate RNA interferance mediated cleavage of its corresponding target region. These design parameters can include destabilization of each end of the siNA construct (see for example Schwarz et al., 2003, Cell, 115, 199-208). Such destabilization can be accomplished for example by using guanosine-cytidine base pairs, alternate base pairs (e.g., wobbles), or destabilizing chemically modified nucleotides at terminal

FIG. 18 shows the chemical structure of a series of modified uridines used herein: (1) 5′-phosphate of 2′-deoxy-2′-C-allyl-uridine; (2): 2′-deoxy-2′-amino uridine; (3): 2′-O-methyl uridine; (4): 2′-deoxy uridine; (5): 2′-O-allyl uridine; (6): 2′-deoxy-2′-propyl uridine; (7): 2′-deoxy-2′-C-allyl cytidine; (8) 2′-deoxy-2′-fluoro uridine.

FIG. 19 shows the inhibition of binding of 1 μg/of CA1USR mAb by increasing concentrations of different nucleotide competitors. The change in binding by CA1USR to 2′-C-allyl U—KLH coupled to DNA-Bind plates with different concentrations of a series of nucleotide competitors in solution is shown. (-□-); 2′-C-allyl U; (-□-): 5′ UMP; (-□-): 3′ UMP; (-×-): 2′ UMP; (-+-): 5′ UTP; (-□-): 2′ deoxy UTP, respectively, were incubated with 1 μg/ml of CA1USR prior to addition of the competitor-antibody mixture to 2′-deoxy-2′-C-allyl Uridine coupled to KLH.

FIG. 20 shows the chemical structure of an ANGIOZYME® enzymatic nucleic acid (SEQ ID NO: 1).

FIG. 21 shows binding of CA1USR to different modified enzymatic nucleic acid molecules. Biotinylated versions of a series of modified enzymatic nucleic acids were bound in equimolar concentrations to streptavidin wells, and binding of 1 μg/ml of purified CA1USR was determined. (-□-) indicates binding of biotinylated enzymatic nucleic acid molecule; (-□-) 2′-deoxy U enzymatic nucleic acid molecule; (-+-): 2′-amino U enzymatic nucleic acid molecule; (-×-): 2′-O Me U enzymatic nucleic acid molecule; (-□-): 2′-O allyl U enzymatic nucleic acid molecule; (-□-): 2′-C-allyl C enzymatic nucleic acid molecule.

FIG. 22 shows the chemical structure of a HERZYME™ enzymatic nucleic acid (SEQ ID NO: 3).

FIG. 23 shows a non-limiting example of selective binding of monoclonal antibody FU1SR to a panel of polynucleotides containing differing chemical modifications including 2′-deoxy-2′-fluoro substituents.

FIG. 24 shows a non-limiting example of selective binding of monoclonal antibody FU2SR to a panel of polynucleotides containing differing chemical modifications including 2′-deoxy-2′-fluoro substituents.

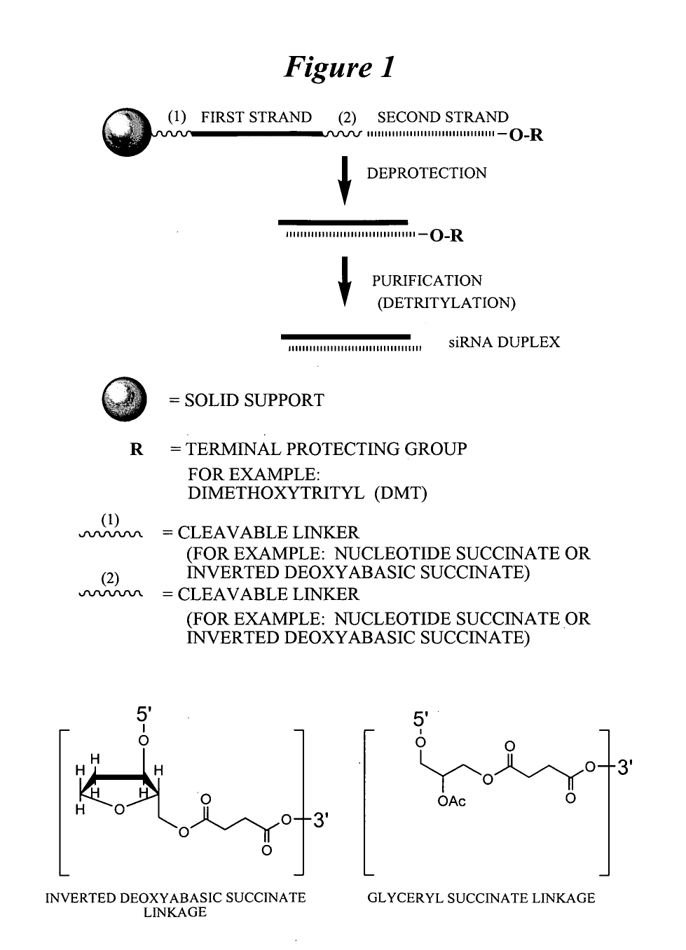

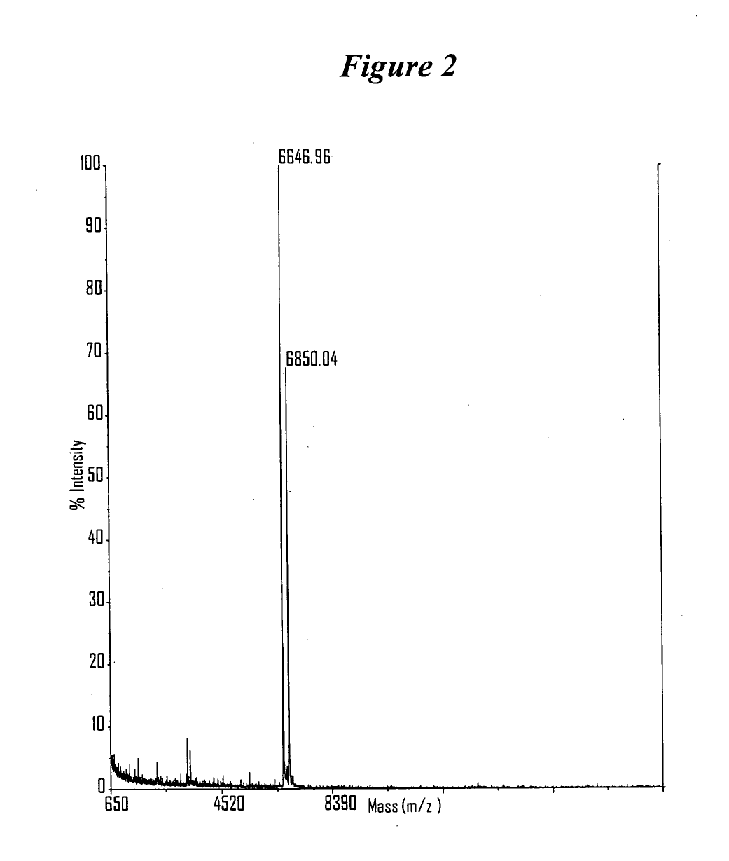

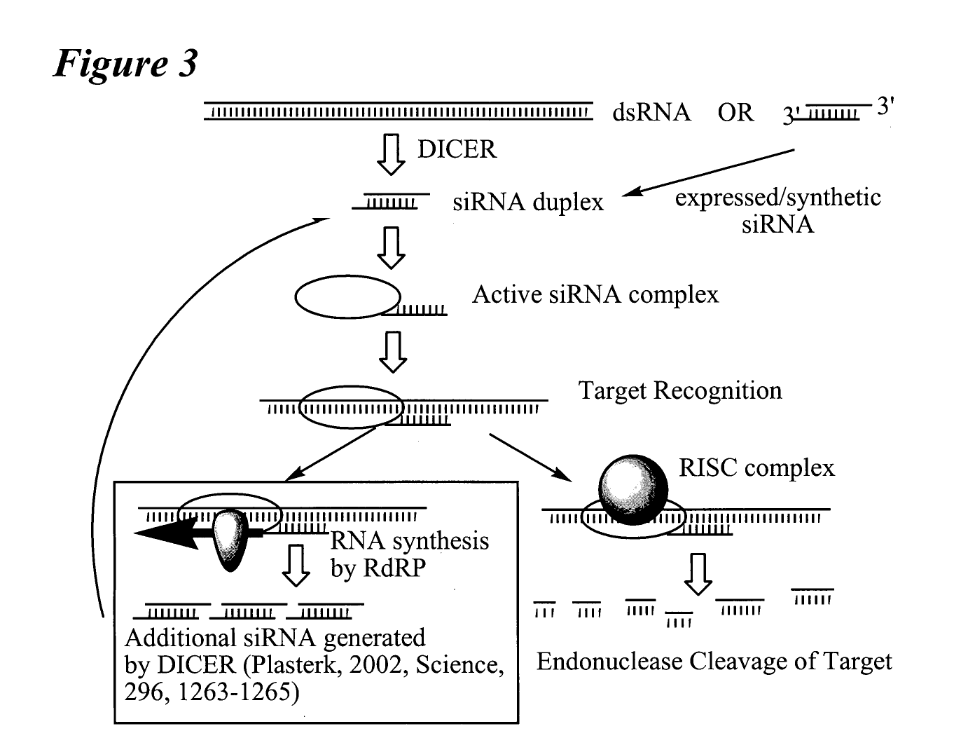

### DETAILED DESCRIPTION OF THE INVENTION

The term “biodegradable nucleic acid linker molecule” as used herein, refers to a nucleic acid molecule that is biodegradable and designed to connect one molecule to another molecule, for example, a biologically active molecule. The stability of the biodegradable nucleic acid linker molecule can be modulated by using various combinations of ribonucleotides, deoxyribonucleotides, and chemically modified nucleotides, for example 2′-O-methyl, 2′-fluoro, 2′-amino, 2′-O-amino, 2′-C-allyl, 2′-O-allyl, and other 2′-modified or base modified nucleotides. The biodegradable nucleic acid linker molecule can be a dimer, trimer, tetramer or longer nucleic acid molecule, for example, an oligonucleotide of about 2, 3, 4, 5, 6, 7, 8, 9, 10, 11, 12, 13, 14, 15, 16, 17, 18, 19, or 20 nucleotides in length, or can comprise a single nucleotide with a phosphorus based linkage, for example, a phosphoramidate or phosphodiester linkage. The biodegradable nucleic acid linker molecule can also comprise nucleic acid backbone, nucleic acid sugar, or nucleic acid base modifications.

The term “biodegradable” as used herein, refers to degradation in a biological system, for example, enzymatic degradation or chemical degradation.

The term “biologically active molecule” as used herein, refers to compounds or molecules that are capable of eliciting or modifying a biological response in a system. Non-limiting examples of biologically active molecules contemplated by the instant invention include therapeutically active molecules such as antibodies, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, siRNA, siNA, dsRNA, allozymes, aptamers, decoys and analogs thereof. Biologically active molecules of the invention can also include molecules capable of modulating the pharmacokinetics and/or pharmacodynamics of other biologically active molecules, for example, lipids and polymers such as polyamines, polyamides, polyethylene glycol and other polyethers.

The term “phospholipid” as used herein, refers to a hydrophobic molecule comprising at least one phosphorus group. For example, a phospholipid can comprise a phosphorus containing group and saturated or unsaturated alkyl group, optionally substituted with OH, COOH, oxo, amine, or substituted or unsubstituted aryl groups.

The term “enzymatic nucleic acid molecule” as used herein refers to a nucleic acid molecule which has complementarity in a substrate binding region to a specified gene target, and also has an enzymatic activity which is active to specifically cleave target RNA. That is, the enzymatic nucleic acid molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% can also be useful in this invention (see for example Werner and Uhlenbeck, 1995, Nucleic Acids Research, 23, 2092-2096; Hammann et al., 1999, Antisense and Nucleic Acid Drug Dev., 9, 25-31). The nucleic acids can be modified at the base, sugar, and/or phosphate groups. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terms describe nucleic acid molecules with enzymatic activity. The specific enzymatic nucleic acid molecules described in the instant application are not limiting of the invention and those skilled in the art will recognize that what is most important in an enzymatic nucleic acid molecule of this invention is that it has a specific substrate binding site which is complementary to one or more of the target nucleic acid regions, and that it has nucleotide sequences within or surrounding that substrate binding site which impart a nucleic acid cleaving and/or ligation activity to the molecule (Cech et al., U.S. Pat. No. 4,987,071; Cech et al., 1988, 260 JAMA 3030).

The term “nucleic acid molecule” as used herein, refers to a molecule having nucleotides. The nucleic acid can be single, double, or multiple stranded and can comprise modified or unmodified nucleotides or non-nucleotides or various mixtures and combinations thereof.

The term “antisense nucleic acid”, as used herein refers to a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to a substrate such that the substrate molecule forms a loop, and/or an antisense molecule can bind such that the antisense molecule forms a loop. Thus, the antisense molecule can be complementary to two or more non-contiguous substrate sequences or two (or more) non-contiguous sequence portions of an antisense molecule can be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49. In addition, antisense DNA can be used to target RNA by means of DNA-RNA interactions, thereby activating RNase H, which digests the target RNA in the duplex. The antisense oligonucleotides can comprise one or more RNAse H activating regions, which are capable of activating RNAse H cleavage of a target RNA. Antisense DNA can be synthesized chemically or expressed via the use of a single stranded DNA expression vector or equivalent thereof.

The term “RNase H activating region” as used herein refers to a region (generally greater than or equal to about 4 nucleotides to about 25 nucleotides in length, preferably from about 5 nucleotides to about 11 nucleotides in length) of a nucleic acid molecule capable of binding to a target RNA to form a non-covalent complex that is recognized by a cellular RNase H enzyme (see for example Arrow et al., US Patent No. 5,849,902; Arrow et al., U.S. Pat. No. 5,989,912). The RNase H enzyme binds to the nucleic acid molecule-target RNA complex and cleaves the target RNA sequence. The RNase H activating region comprises, for example, phosphodiester, phosphorothioate (preferably at least four of the nucleotides are phosphorothioate substitutions; more specifically, 4-11 of the nucleotides are phosphorothioate substitutions); phosphorodithioate, 5′-thiophosphate, or methylphosphonate backbone chemistry or a combination thereof. In addition to one or more backbone chemistries described above, the RNase H activating region can also comprise a variety of sugar chemistries. For example, the RNase H activating region can comprise deoxyribose, arabino, fluoroarabino or a combination thereof, nucleotide sugar chemistry. Those skilled in the art will recognize that the foregoing are non-limiting examples and that any combination of phosphate, sugar and base chemistry of a nucleic acid that supports the activity of RNase H enzyme is within the scope of the definition of the RNase H activating region and the instant invention.

The term “2-5A chimera” as used herein refers to an oligonucleotide containing a 5′-phosphorylated 2′-5′-linked adenylate residue. These chimeras bind to target RNA in a sequence-specific manner and activate a cellular 2-SA-dependent ribonuclease which, in turn, cleaves the target RNA (Torrence et al., 1993 Proc. Natl. Acad. Sci. USA 90, 1300; Silverman et al., 2000, Methods Enzymol., 313, 522-533; Player and Torrence, 1998, Pharmacol. Ther., 78, 55-113).

The term “gene” it as used herein refers to a nucleic acid that encodes an RNA, for example, nucleic acid sequences including, but not limited to, structural genes encoding a polypeptide.

The term “pathogenic protein” as used herein refers to one or more endogenous or exongenous proteins that are associated with a disease state or condition, for example a particular cancer or viral infection.

By “RNA” is meant a molecule comprising at least one ribonucleotide residue. By “ribonucleotide” is meant a nucleotide with a hydroxyl group at the 2′ position of a β-D-ribofuranose moiety. The terms include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siNA or internally, for example at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

The term “decoy” as used herein refers to a nucleic acid molecule or aptamer that is designed to preferentially bind to a predetermined ligand. Such binding can result in the inhibition or activation of a target molecule. The decoy or aptamer can compete with a naturally occurring binding target for the binding of a specific ligand. For example, it has been shown that over-expression of HIV trans-activation response (TAR) RNA acts as a “decoy,” which efficiently binds HIV tat protein, thereby preventing it from binding to TAR sequences present in the HIV RNA (Sullenger et al., 1990, Cell, 63, 601-608). This is but a single example and those in the art will recognize that other embodiments can be readily generated using techniques generally known in the art, see for example Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628. Similarly, a decoy RNA can be designed to bind to a receptor and block the binding of an effector molecule or a decoy RNA can be designed to bind to receptor of interest and prevent interaction with the receptor.

By “aptamer” or “nucleic acid aptamer” as used herein is meant a polynucleotide that binds specifically to a target molecule wherein the nucleic acid molecule has sequence that is distinct from sequence recognized by the target molecule in its natural setting. Alternately, an aptamer can be a nucleic acid molecule that binds to a target molecule where the target molecule does not naturally bind to a nucleic acid. The target molecule can be any molecule of interest. For example, the aptamer can be used to bind to a ligand-binding domain of a protein, thereby preventing interaction of the naturally occurring ligand with the protein. This is a non-limiting example and those in the art will recognize that other embodiments can be readily generated using techniques generally known in the art, see for example Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628.

The term “single stranded RNA” (ssRNA) as used herein refers to a naturally occurring or synthetic ribonucleic acid molecule comprising a linear single strand, for example, a messenger RNA (mRNA), transfer RNA (tRNA), or ribosomal RNA (rRNA).

The term “single stranded DNA” (ssDNA) as used herein refers to a naturally occurring or synthetic deoxyribonucleic acid molecule comprising a linear single strand, for example, a sense or antisense gene sequence or EST (Expressed Sequence Tag).

The term “double stranded RNA” or “dsRNA” as used herein refers to a double stranded RNA molecule capable of RNA interference, including short interfering RNA (siRNA) or short interfering nucleic acid (siNA).

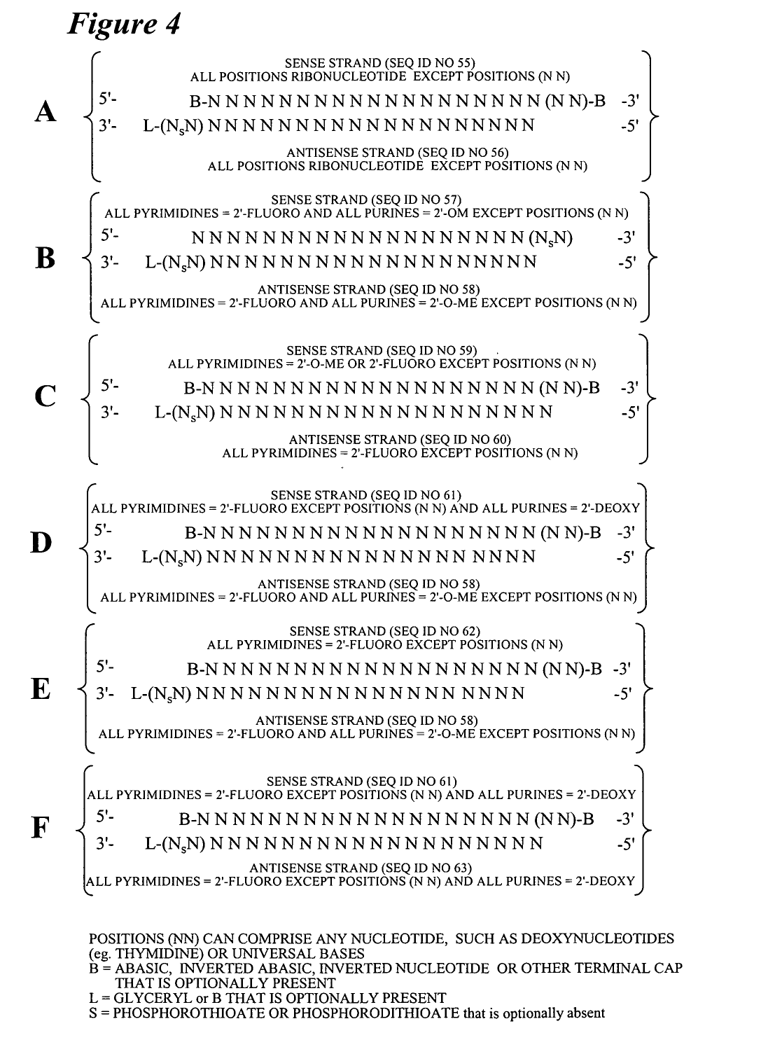

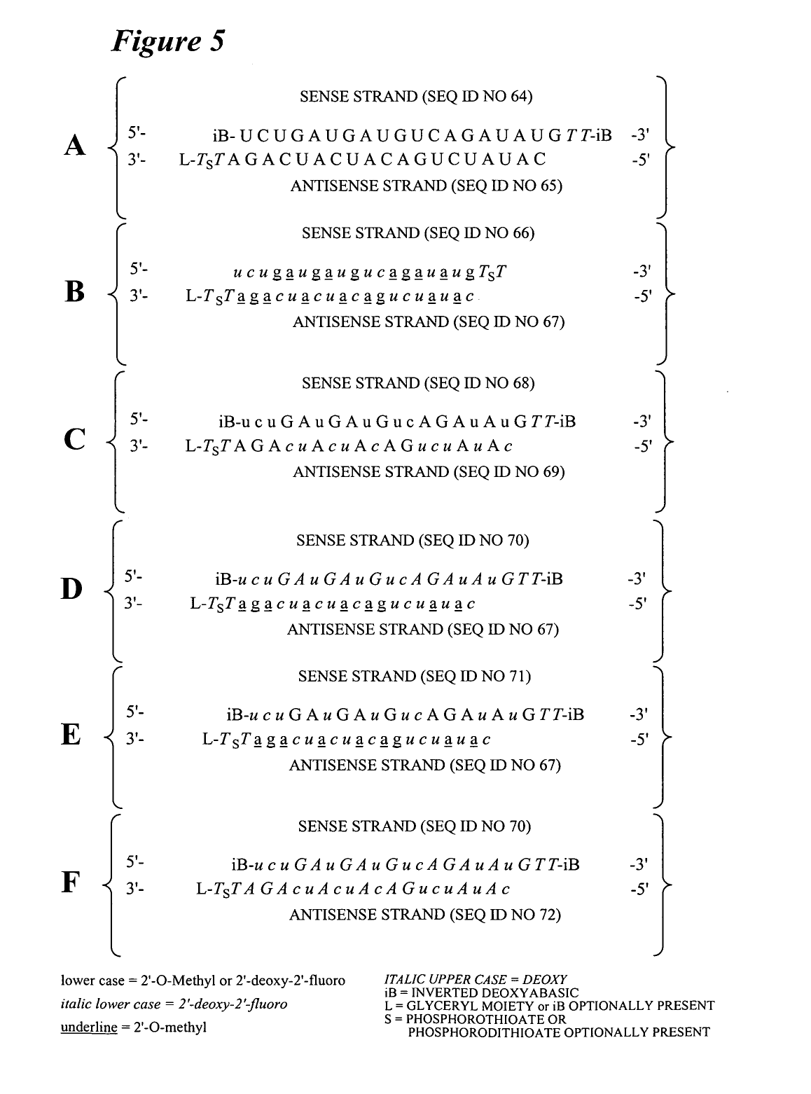

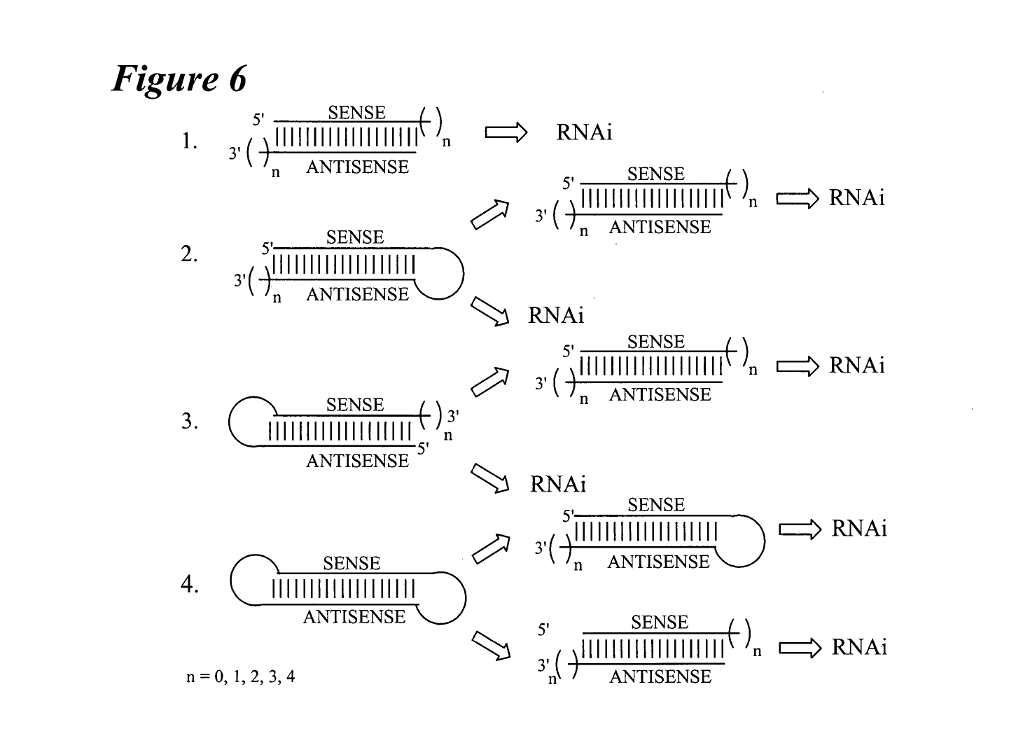

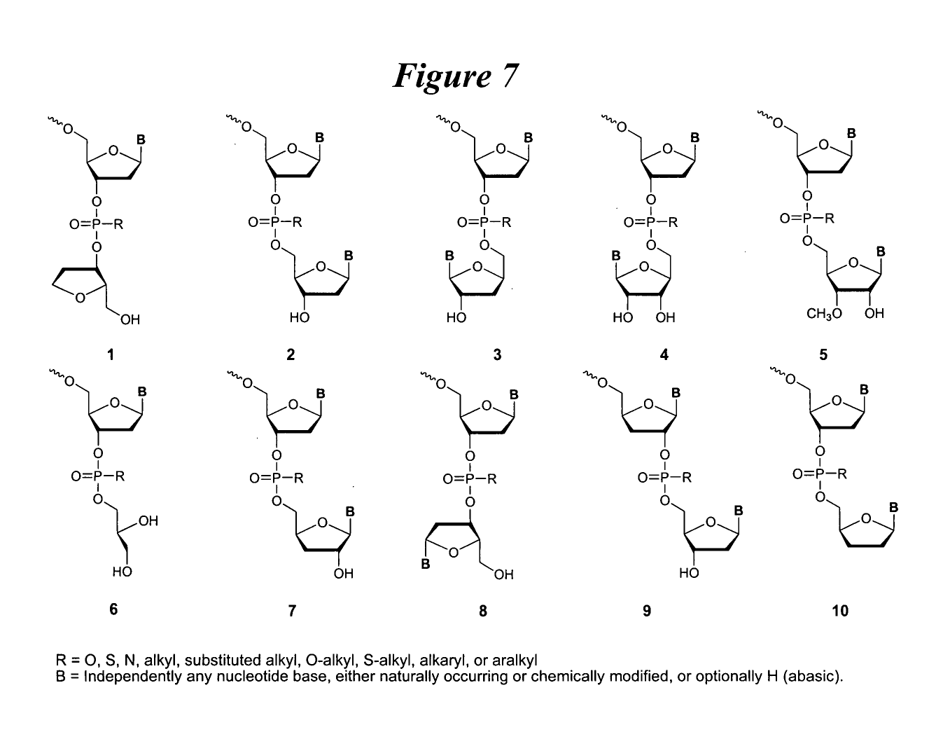

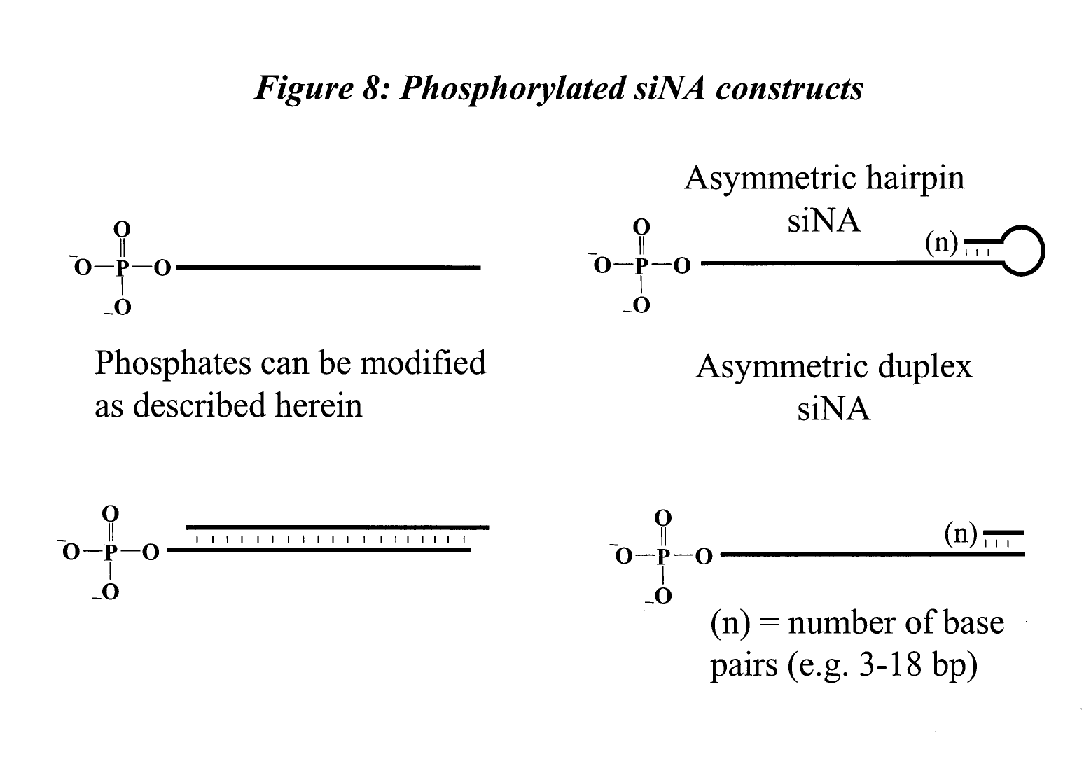

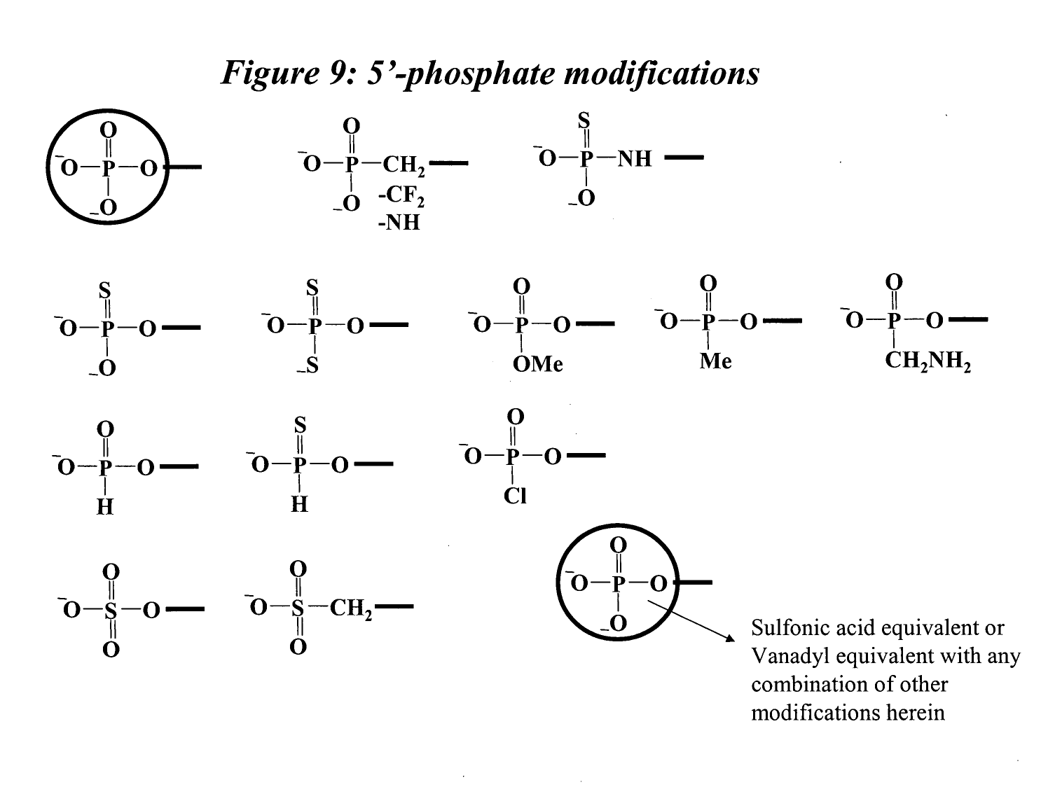

The term “short interfering nucleic acid”, “siNA”, “short interfering RNA”, “siRNA”, “short interfering nucleic acid molecule”, “short interfering oligonucleotide molecule”, or “chemically-modified short interfering nucleic acid molecule” as used herein refers to any nucleic acid molecule capable of inhibiting or down regulating gene expression or viral replication, for example by mediating RNA interference “RNAi” or gene silencing in a sequence-specific manner; see for example Zamore et al., 2000, Cell, 101, 25-33; Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237; Hutvagner and Zamore, 2002, Science, 297, 2056-60; McManus et al., 2002, RNA, 8, 842-850; Reinhart et al., 2002, Gene & Dev., 16, 1616-1626; and Reinhart & Bartel, 2002, Science, 297, 1831). Non limiting examples of siNA molecules of the invention are shown in FIGS. 4-6. For example the siNA can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siNA can be assembled from two separate oligonucleotides, where one strand is the sense strand and the other is the antisense strand, wherein the antisense and sense strands are self-complementary (i.e. each strand comprises nucleotide sequence that is complementary to nucleotide sequence in the other strand; such as where the antisense strand and sense strand form a duplex or double stranded structure, for example wherein the double stranded region is about 19 base pairs); the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof. Alternatively, the siNA is assembled from a single oligonucleotide, where the self-complementary sense and antisense regions of the siNA are linked by means of a nucleic acid based or non-nucleic acid-based linker(s). The siNA can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. The siNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siNA molecule capable of mediating RNAi. The siNA can also comprise a single stranded polynucleotide having nucleotide sequence complementary to nucleotide sequence in a target nucleic acid molecule or a portion thereof (for example, where such siNA molecule does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example Martinez et al., 2002, Cell., 110, 563-574 and Schwarz et al., 2002, Molecular Cell, 10, 537-568), or 5′,3′-diphosphate. In certain embodiments, the siNA molecule of the invention comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der waals interactions, hydrophobic intercations, and/or stacking interactions. In certain embodiments, the siNA molecules of the invention comprise nucleotide sequence that is complementary to nucleotide sequence of a target gene. In another embodiment, the siNA molecule of the invention interacts with nucleotide sequence of a target gene in a manner that causes inhibition of expression of the target gene. As used herein, siNA molecules need not be limited to those molecules containing only RNA, but further encompasses chemically-modified nucleotides and non-nucleotides. In certain embodiments, the short interfering nucleic acid molecules of the invention lack 2′-hydroxy (2′-OH) containing nucleotides. Applicant describes in certain embodiments short interfering nucleic acids that do not require the presence of nucleotides having a 2′-hydroxy group for mediating RNAi and as such, short interfering nucleic acid molecules of the invention optionally do not include any ribonucleotides (e.g., nucleotides having a 2′-OH group). Such siNA molecules that do not require the presence of ribonucleotides within the siNA molecule to support RNAi can however have an attached linker or linkers or other attached or associated groups, moieties, or chains containing one or more nucleotides with 2′-OH groups. Optionally, siNA molecules can comprise ribonucleotides at about 5, 10, 20, 30, 40, or 50% of the nucleotide positions. The modified short interfering nucleic acid molecules of the invention can also be referred to as short interfering modified oligonucleotides “siMON.” As used herein, the term siNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics. For example, siNA molecules of the invention can be used to epigenetically silence genes at both the post-transcriptional level or the pre-transcriptional level. In a non-limiting example, epigenetic regulation of gene expression by siNA molecules of the invention can result from siNA mediated modification of chromatin structure to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237).

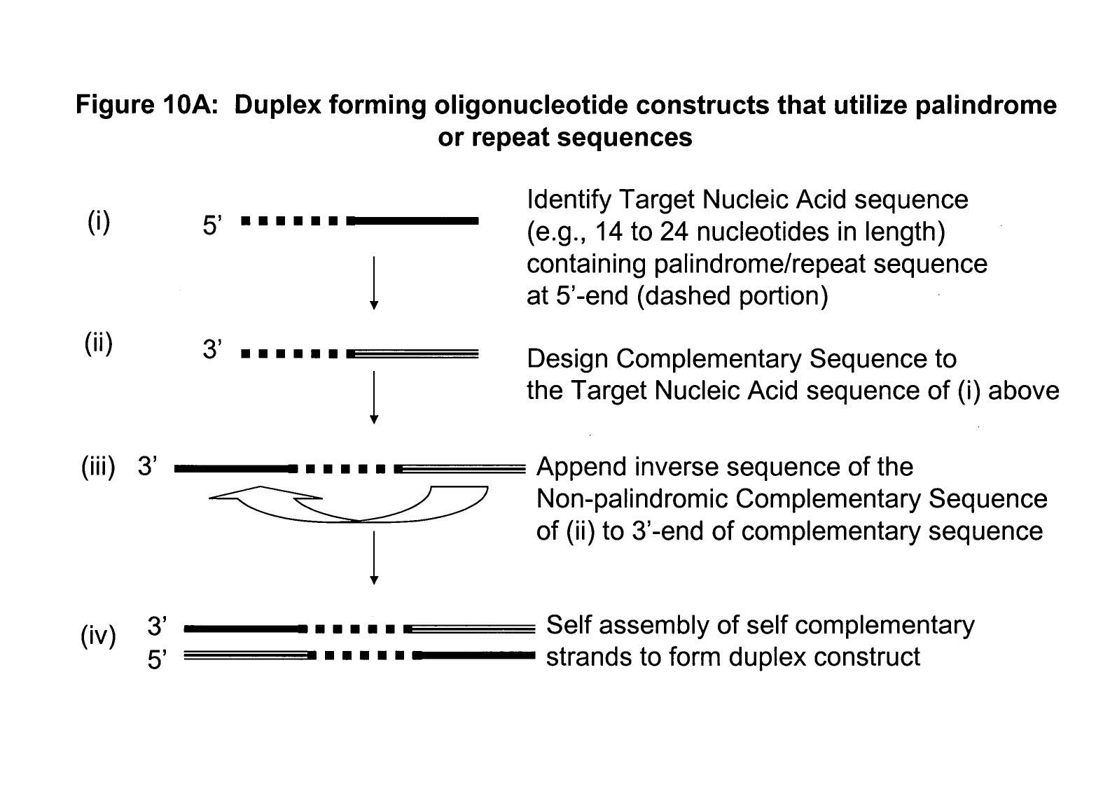

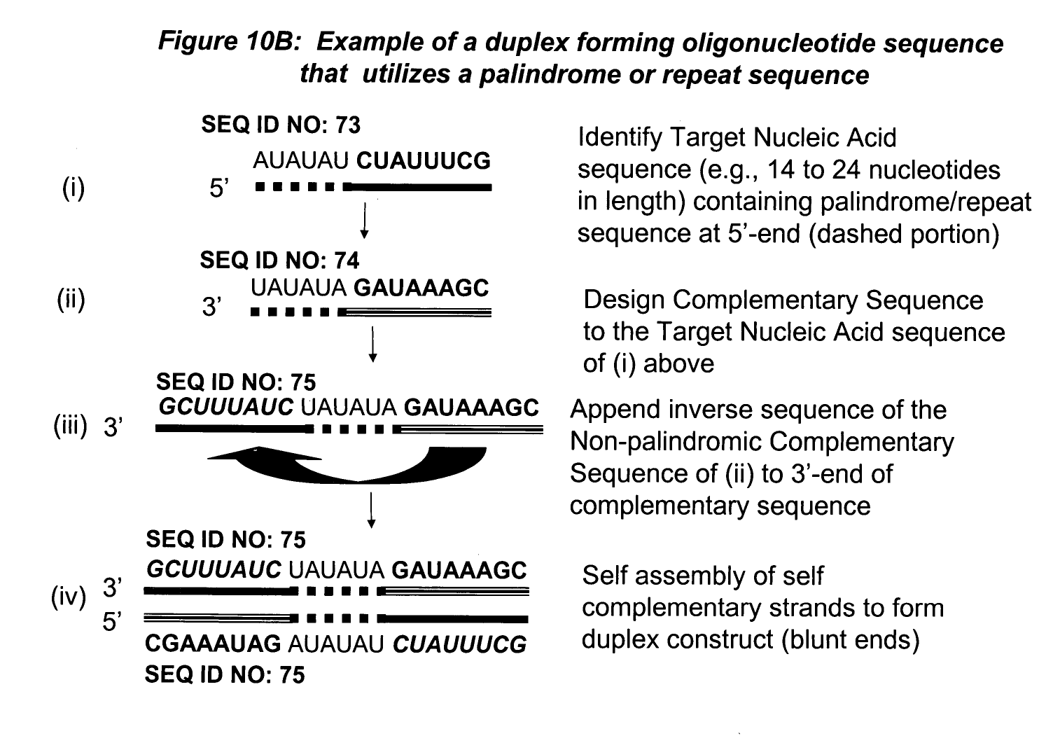

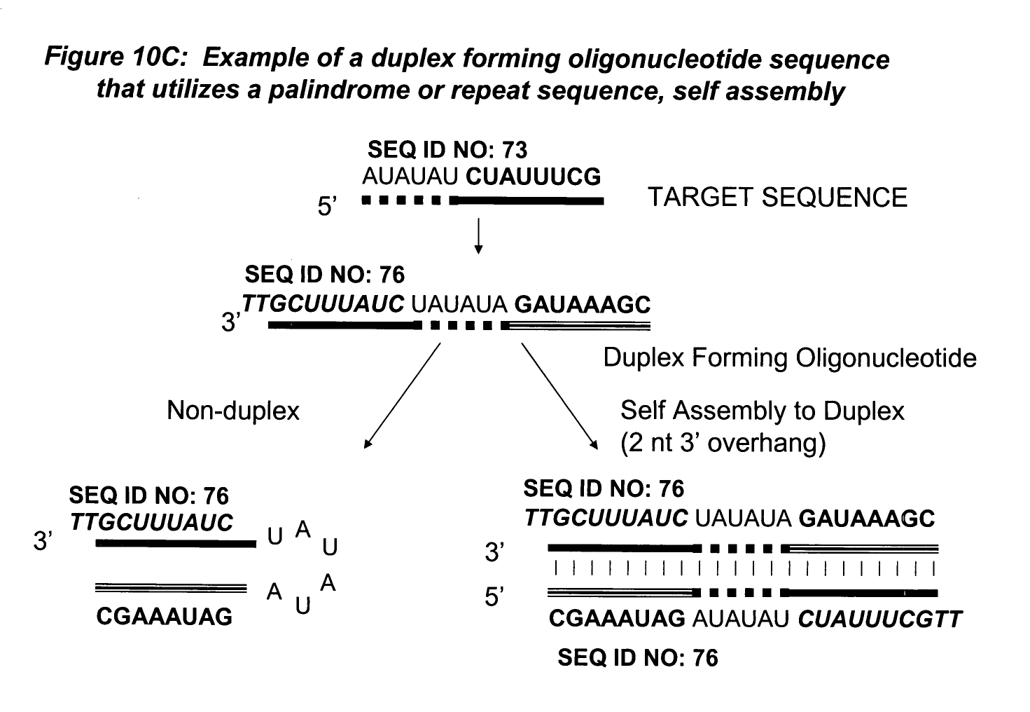

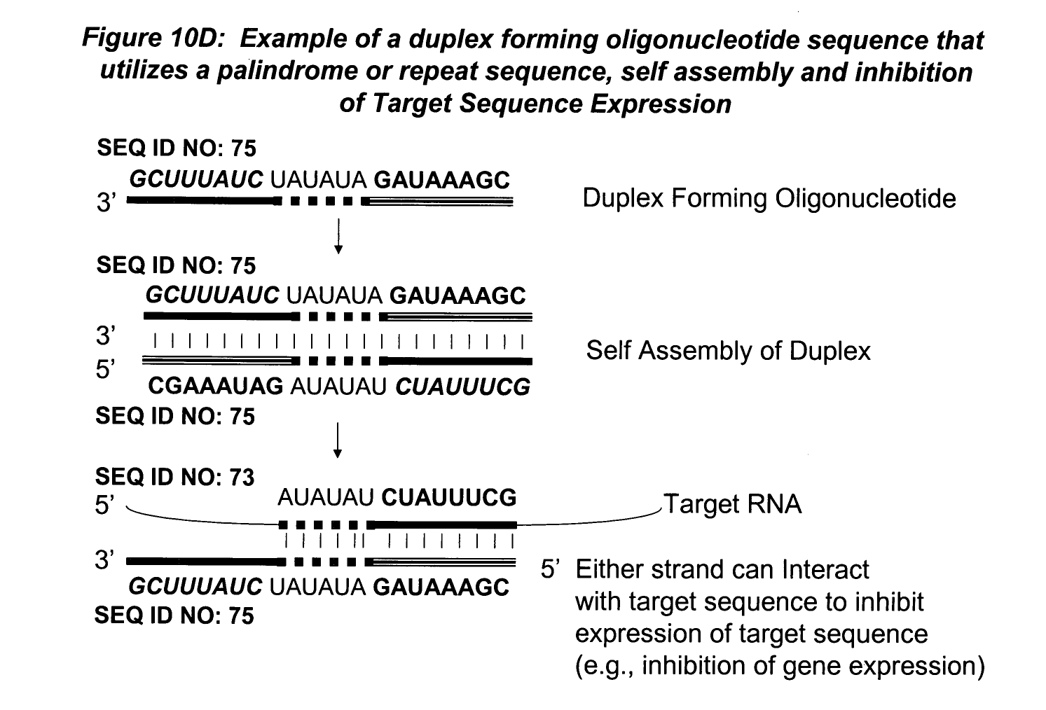

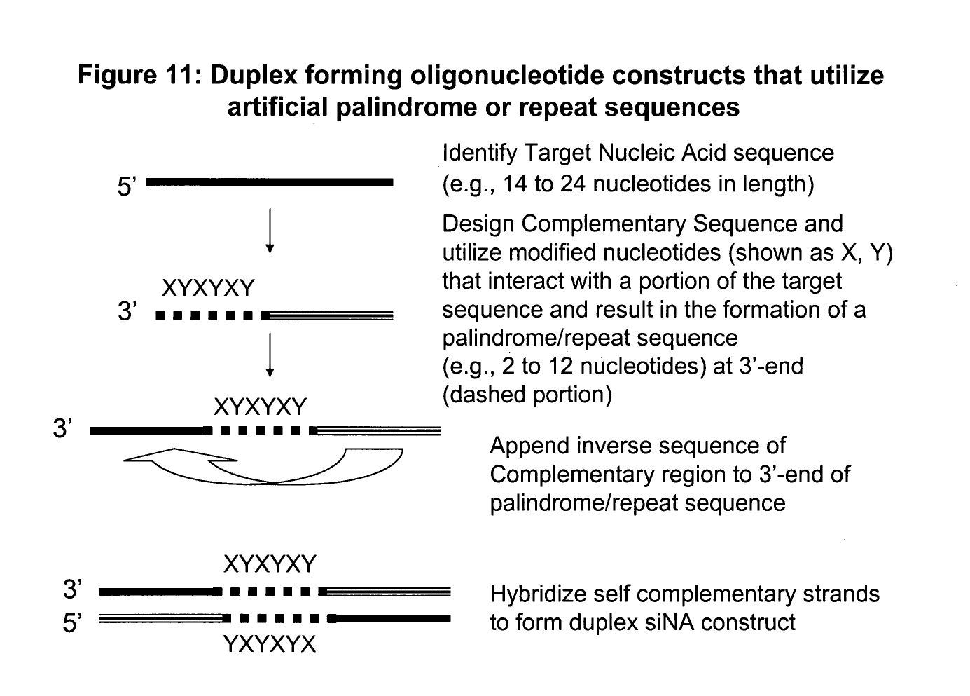

In one embodiment, a siNA molecule of the invention is a duplex forming oligonucleotide “DFO”, (see for example FIGS. 10-11 and Vaish et al., U.S. Ser. No. 10/727,780 filed Dec. 3, 2003).

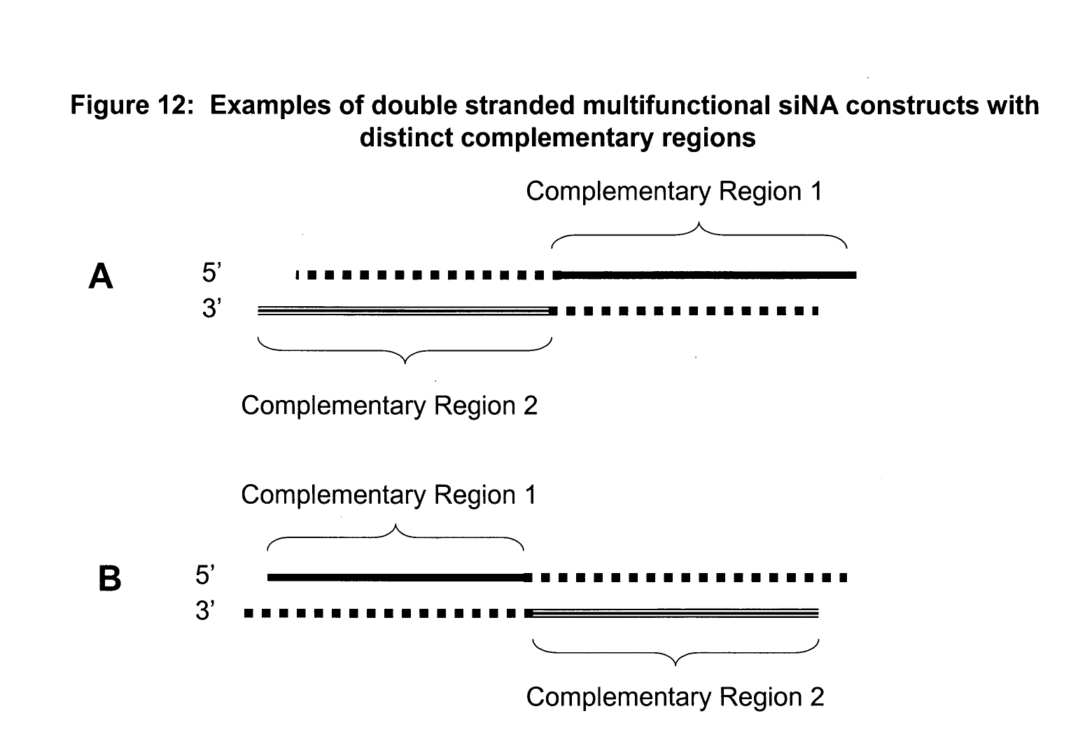

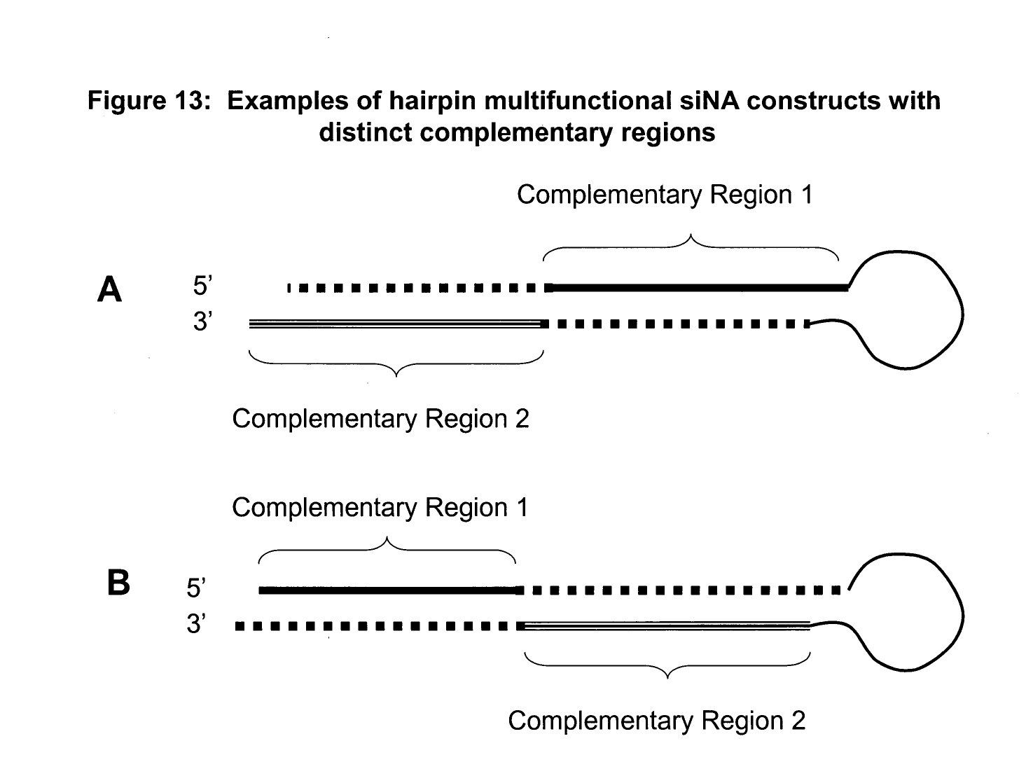

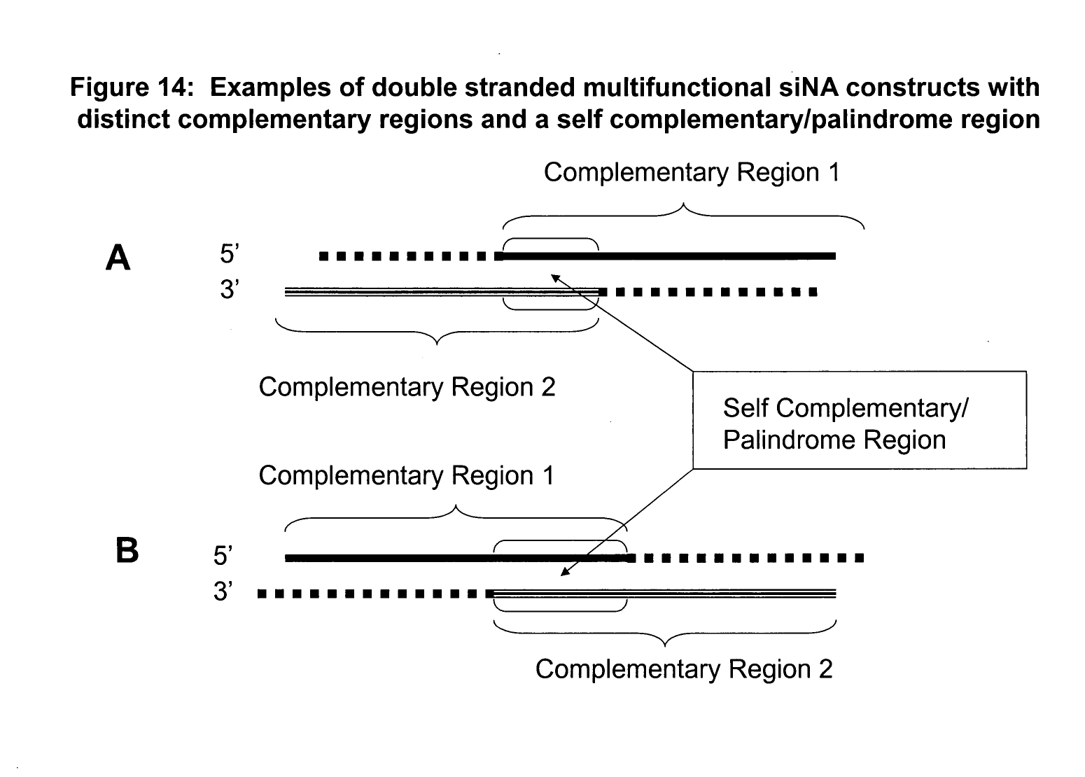

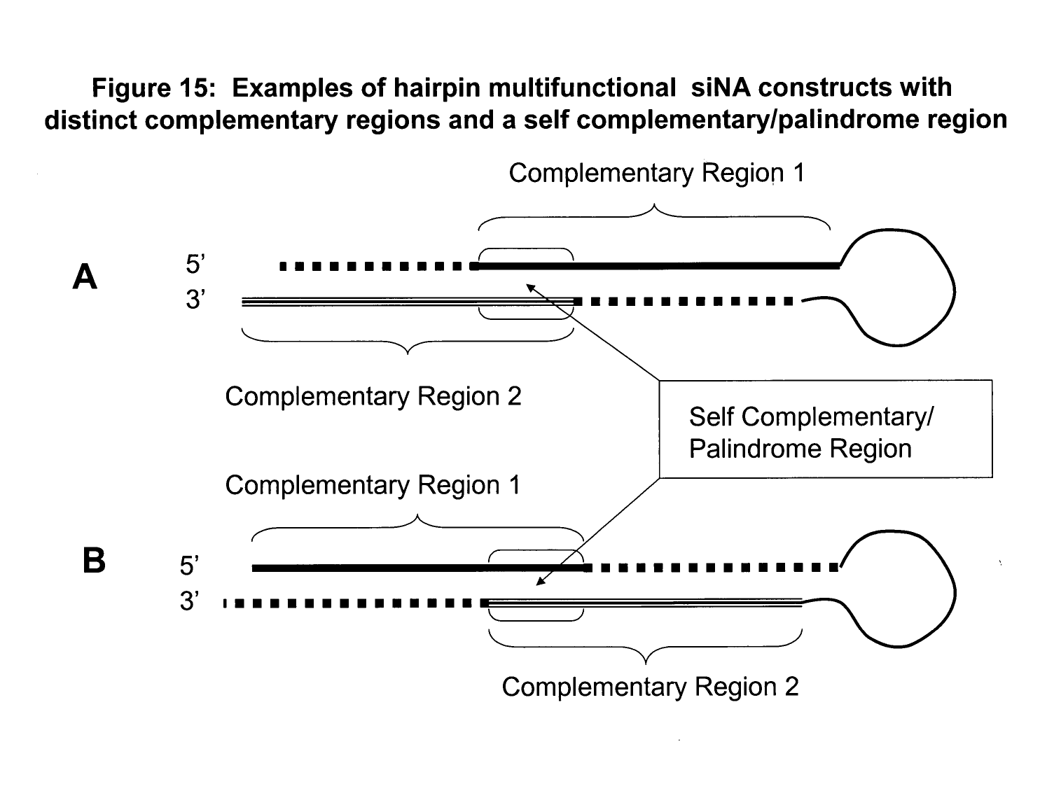

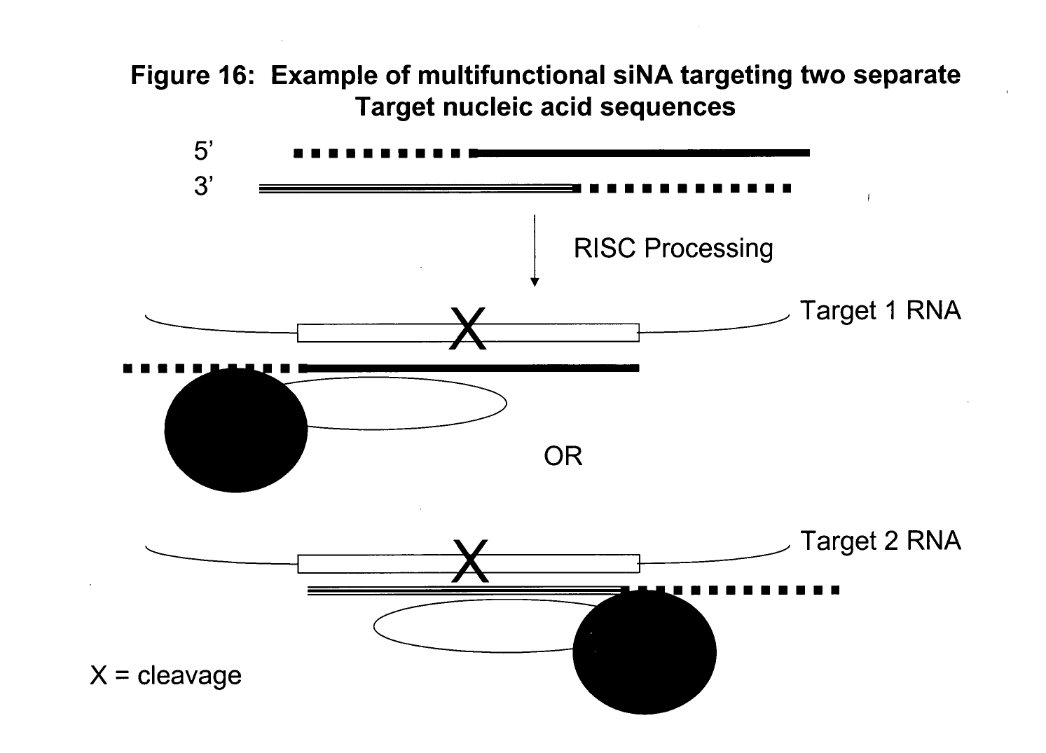

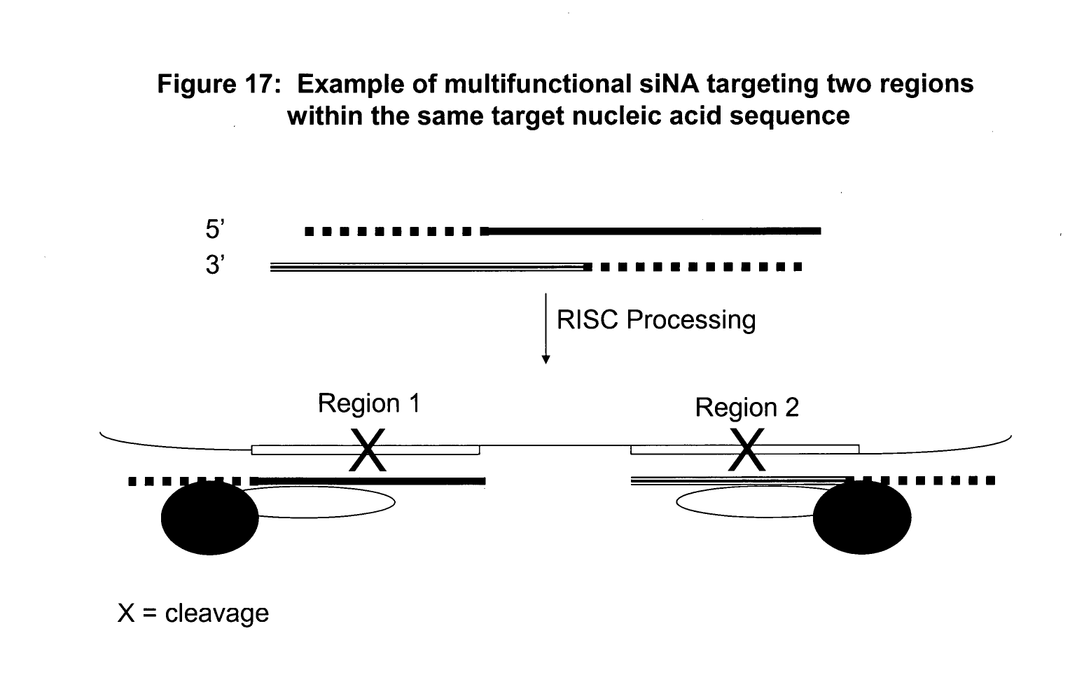

In one embodiment, a siNA molecule of the invention is a multifunctional siNA, (see for example FIGS. 12-18 and Jadhav et al., U.S. Ser. No. 60/543,480 filed Feb. 10, 2004). The multifunctional siNA of the invention can comprise sequence targeting, for example, two regions of of a target RNA.

By “asymmetric hairpin” as used herein is meant a linear siNA molecule comprising an antisense region, a loop portion that can comprise nucleotides or non-nucleotides, and a sense region that comprises fewer nucleotides than the antisense region to the extent that the sense region has enough complementary nucleotides to base pair with the antisense region and form a duplex with loop. For example, an asymmetric hairpin siNA molecule of the invention can comprise an antisense region having length sufficient to mediate RNAi in a cell or in vitro system (e.g. about 19 to about 22 (e.g., about 19, 20, 21, or 22) nucleotides) and a loop region comprising about 4 to about 8 (e.g., about 4, 5, 6, 7, or 8) nucleotides, and a sense region having about 3 to about 18 (e.g., about 3, 4, 5, 6, 7, 8, 9, 10, 11, 12, 13, 14, 15, 16, 17, or 18) nucleotides that are complementary to the antisense region. The asymmetric hairpin siNA molecule can also comprise a 5′-terminal phosphate group that can be chemically modified. The loop portion of the asymmetric hairpin siNA molecule can comprise nucleotides, non-nucleotides, linker molecules, or conjugate molecules as described herein.

By “asymmetric duplex” as used herein is meant a siNA molecule having two separate strands comprising a sense region and an antisense region, wherein the sense region comprises fewer nucleotides than the antisense region to the extent that the sense region has enough complementary nucleotides to base pair with the antisense region and form a duplex. For example, an asymmetric duplex siNA molecule of the invention can comprise an antisense region having length sufficient to mediate RNAi in a cell or in vitro system (e.g. about 19 to about 22 (e.g. about 19, 20, 21, or 22) nucleotides) and a sense region having about 3 to about 18 (e.g., about 3, 4, 5, 6, 7, 8, 9, 10, 11, 12, 13, 14, 15, 16, 17, or 18) nucleotides that are complementary to the antisense region.

By “modulate” is meant that the expression of the gene, or level of RNA molecule or equivalent RNA molecules encoding one or more proteins or protein subunits, or activity of one or more proteins or protein subunits is up regulated or down regulated, such that expression, level, or activity is greater than or less than that observed in the absence of the modulator. For example, the term “modulate” can mean “inhibit,” but the use of the word “modulate” is not limited to this definition.

By “inhibit”, “down-regulate”, or “reduce”, it is meant that the expression of the gene, or level of RNA molecules or equivalent RNA molecules encoding one or more proteins or protein subunits, or activity of one or more proteins or protein subunits, is reduced below that observed in the absence of the nucleic acid molecules (e.g., siNA, siRNA, ssRNA, dsRNA, antisense, aptamers, ribozymes, enzymatic nucleic acids, DNAzymes, decoys, triplex-forming oligonucleotides, 2′,5′-oligoadenylate chimeras) of the invention. In one embodiment, inhibition, down-regulation or reduction with an siNA molecule is below that level observed in the presence of an inactive or attenuated molecule. In another embodiment, inhibition, down-regulation, or reduction with siNA molecules is below that level observed in the presence of, for example, an siNA molecule with scrambled sequence or with mismatches. In another embodiment, inhibition, down-regulation, or reduction of gene expression with a nucleic acid molecule of the instant invention is greater in the presence of the nucleic acid molecule than in its absence.

By “gene”, or “target gene”, is meant, a nucleic acid that encodes an RNA, for example, nucleic acid sequences including, but not limited to, structural genes encoding a polypeptide. A gene or target gene can also encode a functional RNA (fRNA) or non-coding RNA (ncRNA), such as small temporal RNA (stRNA), micro RNA (miRNA), small nuclear RNA (snRNA), short interfering RNA (siRNA), small nucleolar RNA (snRNA), ribosomal RNA (rRNA), transfer RNA (tRNA) and precursor RNAs thereof. Such non-coding RNAs can serve as target nucleic acid molecules for siNA mediated RNA interference in modulating the activity of fRNA or ncRNA involved in functional or regulatory cellular processes. Abberant fRNA or ncRNA activity leading to disease can therefore be modulated by siNA molecules of the invention. siNA molecules targeting fRNA and ncRNA can also be used to manipulate or alter the genotype or phenotype of an organism or cell, by intervening in cellular processes such as genetic imprinting, transcription, translation, or nucleic acid processing (e.g., transamination, methylation etc.). The target gene can be a gene derived from a cell, an endogenous gene, a transgene, or exogenous genes such as genes of a pathogen, for example a virus, which is present in the cell after infection thereof. The cell containing the target gene can be derived from or contained in any organism, for example a plant, animal, protozoan, virus, bacterium, or fungus. Non-limiting examples of plants include monocots, dicots, or gymnosperms. Non-limiting examples of animals include vertebrates or invertebrates. Non-limiting examples of fungi include molds or yeasts.

The term “allozyme” as used herein refers to an allosteric enzymatic nucleic acid molecule, see for example George et al., U.S. Pat. Nos. 5,834,186 and 5,741,679, Shih et al., U.S. Pat. No. 5,589,332, Nathan et al., U.S. Pat. No. 5,871,914, all of which are herein incorporated by reference in their entireties; Nathan and Ellington, International PCT publication No. WO 00/24931, Breaker et al., International PCT Publication Nos. WO 00/26226 and 98/27104, and Sullenger et al., International PCT publication No. WO 99/29842. The term “2-5A chimera” as used herein refers to an oligonucleotide containing a 5′-phosphorylated 2′-5′-linked adenylate residue. These chimeras bind to target RNA in a sequence-specific manner and activate a cellular 2-5A-dependent ribonuclease which, in turn, cleaves the target RNA (see for example Torrence et al., 1993 Proc. Natl. Acad. Sci. USA 90, 1300; Silverman et al., 2000, Methods Enzymol., 313, 522-533; Player and Torrence, 1998, Pharmacol. Ther., 78, 55-113).

The term “triplex forming oligonucleotides” as used herein refers to an oligonucleotide that binds to a double-stranded DNA in a sequence-specific manner to form a triple-strand helix. Formation of such triple helix structure has been shown to inhibit transcription of the targeted gene (see for example Duval-Valentin et al., 1992 Proc. Natl. Acad. Sci. USA 89, 504; Fox, 2000, Curr. Med. Chem., 7, 17-37; Praseuth et. al., 2000, Biochim. Biophys. Acta, 1489, 181-206).

By “proliferative disease” or “cancer” as used herein is meant, any disease or condition characterized by unregulated cell growth or replication as is known in the art; including breast cancer, cancers of the head and neck, including various lymphomas such as mantle cell lymphoma, non-Hodgkins lymphoma, adenoma, squamous cell carcinoma, laryngeal carcinoma, cancers of the retina, cancers of the esophagus, multiple myeloma, ovarian cancer, uterine cancer, melanoma, colorectal cancer, lung cancer, bladder cancer, prostate cancer, glioblastoma, lung cancer (including non-small cell lung carcinoma), pancreatic cancer, cervical cancer, head and neck cancer, skin cancers, nasopharyngeal carcinoma, liposarcoma, epithelial carcinoma, renal cell carcinoma, gallbladder adeno carcinoma, parotid adenocarcinoma, endometrial sarcoma, multidrug resistant cancers; and proliferative diseases and conditions, such as neovascularization associated with tumor angiogenesis, macular degeneration (e.g., wet/dry AMD), corneal neovascularization, diabetic retinopathy, neovascular glaucoma, myopic degeneration and other proliferative diseases and conditions such as restenosis and polycystic kidney disease, and any other cancer or proliferative disease or condition that can respond to the level of a target nucleic acid in a cell or tissue, alone or in combination with other therapies.

By “inflammatory disease” or “inflammatory condition” as used herein is meant any disease or condition characterized by an inflammatory or allergic process as is known in the art, such as inflammation, acute inflammation, chronic inflammation, atherosclerosis, restenosis, asthma, allergic rhinitis, atopic dermatitis, septic shock, rheumatoid arthritis, inflammatory bowl disease, inflammotory pelvic disease, pain, ocular inflammatory disease, celiac disease, Leigh Syndrome, Glycerol Kinase Deficiency, Familial eosinophilia (FE), autosomal recessive spastic ataxia, laryngeal inflammatory disease; Tuberculosis, Chronic cholecystitis, Bronchiectasis, Silicosis and other pneumoconioses, and any other inflammatory disease or condition that can respond to the level of a target nucleic acid in a cell or tissue, alone or in combination with other therapies.

By “autoimmune disease” or “autoimmune condition” as used herein is meant, any disease or condition characterized by autoimmunity as is known in the art, such as multiple sclerosis, diabetes mellitus, lupus, celiac disease, Crohn's disease, ulcerative colitis, Guillain-Barre syndrome, scleroderms, Goodpasture's syndrome, Wegener's granulomatosis, autoimmune epilepsy, Rasmussen's encephalitis, Primary biliary sclerosis, Sclerosing cholangitis, Autoimmune hepatitis Addison's disease, Hashimoto's thyroiditis, fibromyalgia, Menier's syndrome; and transplantation rejection (e.g., prevention of allograft rejection) and any other autoimmune disease or condition that can respond to the level of a target nucleic acid in a cell or tissue, alone or in combination with other therapies.

By “homologous sequence” is meant, a nucleotide sequence that is shared by one or more polynucleotide sequences, such as genes, gene transcripts and/or non-coding polynucleotides. For example, a homologous sequence can be a nucleotide sequence that is shared by two or more genes encoding related but different proteins, such as different members of a gene family, different protein epitopes, different protein isoforms or completely divergent genes, such as a cytokine and its corresponding receptors. A homologous sequence can be a nucleotide sequence that is shared by two or more non-coding polynucleotides, such as noncoding DNA or RNA, regulatory sequences, introns, and sites of transcriptional control or regulation. Homologous sequences can also include conserved sequence regions shared by more than one polynucleotide sequence. Homology does not need to be perfect homology (e.g., 100%), as partially homologous sequences are also contemplated by the instant invention (e.g., 99%, 98%, 97%, 96%, 95%, 94%, 93%, 92%, 91%, 90%, 89%, 88%, 87%, 86%, 85%, 84%, 83%, 82%, 81%, 80% etc.).

By “conserved sequence region” is meant, a nucleotide sequence of one or more regions in a polynucleotide does not vary significantly between generations or from one biological system or organism to another biological system or organism. The polynucleotide can include both coding and non-coding DNA and RNA.

By “sense region” is meant a nucleotide sequence of a siNA molecule having complementarity to an antisense region of the siNA molecule. In addition, the sense region of a siNA molecule can comprise a nucleic acid sequence having homology with a target nucleic acid sequence.

By “antisense region” is meant a nucleotide sequence of a siNA molecule having complementarity to a target nucleic acid sequence. In addition, the antisense region of a siNA molecule can optionally comprise a nucleic acid sequence having complementarity to a sense region of the siNA molecule.

By “target nucleic acid” is meant any nucleic acid sequence to be detected in a system or whose expression or activity is to be modulated. The target nucleic acid can be DNA or RNA.

By “complementarity” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonuelcotide being based paired to a second nucleic acid sequence having 10 nucleotides represents 50%, 60%, 70%, 80%, 90%, and 100% complementary respectively). “Perfectly complementary” means that all the contiguous residues of a nucleic acid sequence will hydrogen bond with the same number of contiguous residues in a second nucleic acid sequence.

The term “biological sample” as used herein refers to any sample derived from a biological source, such as a cell, tissue, organism, or component thereof as is known in the art. Non-limiting examples of biological samples include biopsy, tissue, cells, blood, serum, plasma, cerebral spinal fluid, urine, stool, sputum, semen, synovial fluid, hair, skin, and the like.

The term “cell” as used herein refers to its usual biological sense, and does not refer to an entire multicellular organism. A cell can be in vitro, e.g., in cell culture, or present in a multicellular organism, including, e.g., birds, plants and mammals such as humans, cows, sheep, apes, monkeys, swine, dogs, and cats. A cell can be prokaryotic (e.g., a bacterial cell) or eukaryotic (e.g., a mammalian or plant cell).

The term “non-nucleotide” as used herein refers to any group or compound which can be incorporated into a nucleic acid chain in place of one or more nucleotide units, including either sugar and/or phosphate substitutions, allowing the remaining bases to exhibit their enzymatic activity. The group or compound is abasic in that it does not contain a commonly recognized nucleotide base, such as adenosine, guanine, cytosine, uracil or thynine.

The term “nucleotide” as used herein, refers to a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and the like (see for example, Usman et al., U.S. Pat. No. 5,767,263; Eckstein et al., U.S. Pat. No. 5,672,695; Usman et al., U.S. Pat. No. 6,140,491; all hereby incorporated herein by reference). There are several examples of modified nucleic acid bases known in the art as summarized by Limbach et al., 1994, Nucleic Acids Res. 22, 2183. Some non-limiting examples of chemically modified and other natural nucleic acid bases that can be introduced into nucleic acids include, for example, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g., 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and the like (see for example Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

The term “nucleoside” as used herein refers to a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, modified nucleosides, non-natural nucleosides, non-standard nucleosides and the like (see for example, Usman et al., U.S. Pat. No. 5,767,263; Eckstein et al., U.S. Pat. No. 5,672,695; Usman et al., U.S. Pat. No. 6,140,491;; all hereby incorporated herein by reference in their entirety). There are several examples of modified nucleic acid bases known in the art as summarized by Limbach et al., 1994, Nucleic Acids Res. 22, 2183. Some non-limiting examples of chemically modified and other natural nucleic acid bases that can be introduced into nucleic acids include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g., 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and the like (see for example Burgin et al., 1996, Biochemistry, 35, 14090; Uhlman & Peyman, supra). By “modified bases” in this aspect is meant nucleoside bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

The term “cap structure” as used herein, refers to chemical modifications, which have been incorporated at either terminus of the oligonucleotide (see for example Wincott et al., WO 97/26270, incorporated herein by reference in its entirety ). These terminal modifications protect the nucleic acid molecule from exonuclease degradation, and help in delivery and/or localization within a cell. The cap can be present at the 5′-terminus (5′-cap) or at the 3′-terminus (3′-cap) or can be present on both termini. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated herein by reference in its entirety).

The term “abasic” as used herein refers to sugar moieties lacking a base or having other chemical groups in place of a base at the 1′ position, for example a 3′,3′-linked or 5′,5′-linked deoxyabasic ribose derivative (for more details see Adamic et al., U.S. Pat. No. 5,998,203, incorporated herein by reference in its entirety).

The term “unmodified nucleoside” as used herein refers to one of the bases adenine, cytosine, guanine, thymine, uracil joined to the 1′ carbon of β-D-ribo-furanose.

The term “modified nucleoside” as used herein refers to any nucleotide base which contains a modification in the chemical structure of an unmodified nucleotide base, sugar and/or phosphate.

The term “carrier protein” as used herein, refers to any protein that is used as a scaffold for introducing an antigen during immunization. Non-limiting examples of carrier proteins include bovine serum albumin (BSA) or keyhole limpet hemocyanin.

The term “patient” or “subject” as used herein, refers to an organism, which is a donor or recipient of explanted cells or the cells themselves. “Patient” or “subject” also refers to an organism to which the nucleic acid molecules of the invention can be administered, for example, a mammal or mammalian cells, including a human or human cells.

The term “SJL mouse” as used herein refers to a mouse designated S (Swiss Webster) J(Jackson Labs) L (Lambert) or an equivalent strain of mouse highly susceptible to autoimmune disorders and suitable for raising antibodies.

The term “immunizing” as used herein refers to the process of administering an antigen to a suitable mammal for the purpose of generating antibodies to the antigen.

The term “fusion screen” or “fusion screening assay” as used herein refers to a method of screening for antibodies generated from a hybridoma fusion, or screening of antibodies generated via fusion of a lymphocyte or splenocyte to a myeloma, for example ELISA assay.

The term “hybridoma” as used herein refers to a cell that is created by fusing two cells, a secreting cell from the immune system, such as a B-cell, and an immortal cell, such as a myeloma, within a single membrane. The resulting hybrid cell can be cloned, producing identical daughter cells. Each of these daughter clones can secrete cellular products of the immune cell over several generations.

The term “immortal cell” refers to a cell or cell line that can be passaged in cell culture for several generations or indefinitely.

The term “coupling” as used herein refers to a reaction, either chemical or enzymatic, in which one atom, moiety, group, compound or molecule is joined to another atom, moiety, group, compound or molecule.

The term “linker molecule” as used herein refers to any diradical molecule that can connect one portion or component of a compound to another portion or component of the compound. Linkers can be of varying molecular weight, chemical composition, and/or length.

The term “degradable linker” or “cleavable linker” as used herein refers to linker moieties that are capable of cleavage under various conditions. Conditions suitable for cleavage include, but are not limited to, pH, UV irradiation, enzymatic activity, temperature, hydrolysis, elimination and substitution reactions, thermodynamic properties of the linkage, and the like.

The term “degradable nucleic acid linker” as used herein refers to degradable linkers comprising nucleic acids or oligonucleotides that are susceptible to chemical or enzymatic degradation, for example an oligoribonucleotide. The specific degree of degradability of the linker can be modulated by combining chemically modified nucleotides with naturally occurring nucleotides or by varying the number of pyrimidine nucleotides to purine nucleotides.

The term “photolabile linker” as used herein refers to linker moieties known in the art that are selectively cleaved under particular UV wavelengths. Compounds of the invention containing photolabile linkers can be used to deliver compounds to a target cell or tissue of interest and can be subsequently released in the presence of a UV source.

The term “nucleic acid conjugates” as used herein refers to nucleoside, nucleotide and oligonucleotide conjugates.

The term “monoclonal antibody conjugate” as used herein refers to any conjugate molecule comprising a monoclonal antibody coupled to another molecule, such as an nucleic acid molecule, polynucleotide, oligonucleotide, amino acid, peptide, polypeptide, lipid, phospholipid, or small molecule etc.

The term “lipid aggregate” as used herein refers to a lipid-containing composition, wherein the lipid is in the form of a liposome, micelle (non-lamellar phase) or other aggregates with one or more lipids.

The term “biological system” as used herein can be a eukaryotic system or a prokaryotic system, can be comprised of one or more bacterial cells, plant cells or mammalian cells, and extracts or lysates thereof. The system can be of plant origin, mammalian origin, yeast origin, Drosophila origin, or archebacterial origin.

The term “pharmacological composition” or “pharmaceutical formulation” refers to a composition or formulation in a form suitable for administration, for example, systemic administration, into a cell or patient, preferably a human. Suitable forms depend, in part, upon the use or the route of entry, for example oral, transdermal, or by injection. Such forms should not prevent the composition or formulation to reach a target cell (i.e., a cell to which the negatively charged polymer is targeted).

Other features and advantages of the invention will be apparent from the following description of the preferred embodiments and from the claims.

Administration

The compounds and compositions (e.g., nucleic acid molecule such as siNA or antibody complex thereof) of the invention can be adapted for use to treat, for example, variety of disease and conditions such as proliferative diseases and conditions and/or cancer including breast cancer, cancers of the head and neck including various lymphomas such as mantle cell lymphoma, non-Hodgkins lymphoma, adenoma, squamous cell carcinoma, laryngeal carcinoma, cancers of the retina, cancers of the esophagus, multiple myeloma, ovarian cancer, uterine cancer, melanoma, colorectal cancer, lung cancer, bladder cancer, prostate cancer, glioblastoma, lung cancer (including non-small cell lung carcinoma), pancreatic cancer, cervical cancer, head and neck cancer, skin cancers, nasopharyngeal carcinoma, liposarcoma, epithelial carcinoma, renal cell carcinoma, gallbladder adeno carcinoma, parotid adenocarcinoma, endometrial sarcoma, multidrug resistant cancers; and proliferative diseases and conditions, such as neovascularization associated with tumor angiogenesis, macular degeneration (e.g., wet/dry AMD), corneal neovascularization, diabetic retinopathy, neovascular glaucoma, myopic degeneration and other proliferative diseases and conditions such as restenosis and polycystic kidney disease,; inflammatory diseases and conditions such as inflammation, acute inflammation, chronic inflammation, atherosclerosis, restenosis, asthma, allergic rhinitis, atopic dermatitis, septic shock, rheumatoid arthritis, inflammatory bowl disease, inflammotory pelvic disease, pain, ocular inflammatory disease, celiac disease, Leigh Syndrome, Glycerol Kinase Deficiency, Familial eosinophilia (FE), autosomal recessive spastic ataxia, laryngeal inflammatory disease; Tuberculosis, Chronic cholecystitis, Bronchiectasis, Silicosis and other pneumoconioses; autoimmune diseases and conditions such as multiple sclerosis, diabetes mellitus, lupus, celiac disease, Crohn's disease, ulcerative colitis, Guillain-Barre syndrome, scleroderms, Goodpasture's syndrome, Wegener's granulomatosis, autoimmune epilepsy, Rasmussen's encephalitis, Primary biliary sclerosis, Sclerosing cholangitis, Autoimmune hepatitis Addison's disease, Hashimoto's thyroiditis, fibromyalgia, Menier's syndrome; and transplantation rejection (e.g., prevention of allograft rejection) and any other diseases or conditions that are related to or will respond to the levels of gene expression in a cell or tissue, alone or in combination with other therapies.

For example, a siNA molecule can comprise a delivery vehicle, including liposomes, for administration to a subject; carriers and diluents and their salts; and/or can be present in pharmaceutically acceptable formulations. Methods for the delivery of nucleic acid molecules are generally known in the art and are, for example, described in Akhtar et al., 1992, Trends Cell Bio., 2, 139; Delivery Strategies for Antisense Oligonucleotide Therapeutics, ed. Akhtar, 1995, Maurer et al., 1999, Mol. Membr. Biol., 16, 129-140; Hofland and Huang, 1999, Handb. Exp. Pharmacol., 137, 165-192; and Lee et al., 2000, ACS Symp. Ser., 752, 184-192, all of which are incorporated herein by reference. Beigelman et al., U.S. Pat. No. 6,395,713 and Sullivan et al., PCT WO 94/02595 further describe the general methods for delivery of nucleic acid molecules. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis, or by incorporation into other vehicles, such as biodegradable polymers, hydrogels, cyclodextrins (see for example Gonzalez et al., 1999, Bioconjugate Chem., 10, 1068-1074; Wang et al., International PCT publication Nos. WO 03/47518 and WO 03/46185), poly(lactic-co-glycolic)acid (PLGA) and PLCA microspheres (see for example U.S. Pat. No. 6,447,796 and U.S. patent application Publication No. US 2002130430), biodegradable nanocapsules, and bioadhesive microspheres, or by proteinaceous vectors (O'Hare and Normand, International PCT Publication No. WO 00/53722).

In one embodiment, the nucleic acid molecules of the invention can also be formulated or complexed with polyethyleneimine and derivatives thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives. Alternatively, the nucleic acid/vehicle combination is locally delivered by direct injection or by use of an infusion pump.

In one embodiment, the compounds and compositions the invention are administered to the CNS. Experiments have demonstrated the efficient in vivo uptake of nucleic acids by neurons. As an example of local administration of nucleic acids to nerve cells, Sommer et al., 1998, Antisense Nuc. Acid Drug Dev., 8, 75, describe a study in which a 15 mer phosphorothioate antisense nucleic acid molecule to c-fos is administered to rats via microinjection into the brain. Antisense molecules labeled with tetramethylrhodamine-isothiocyanate (TRITC) or fluorescein isothiocyanate (FITC) were taken up by exclusively by neurons thirty minutes post-injection. A diffuse cytoplasmic staining and nuclear staining was observed in these cells. As an example of systemic administration of nucleic acid to nerve cells, Epa et al., 2000, Antisense Nuc. Acid Drug Dev., 10, 469, describe an in vivo mouse study in which beta-cyclodextrin-adamantane-oligonucleotide conjugates were used to target the p75 neurotrophin receptor in neuronally differentiated PC12 cells. Following a two week course of IP administration, pronounced uptake of p75 neurotrophin receptor antisense was observed in dorsal root ganglion (DRG) cells. In addition, a marked and consistent down-regulation of p75 was observed in DRG neurons. Additional approaches to the targeting of nucleic acid to neurons are described in Broaddus et al., 1998, J. Neurosurg., 88(4), 734; Karle et al., 1997, Eur. J. Pharmocol., 340(2/3), 153; Bannai et al., 1998, Brain Research, 784(1,2), 304; Rajakumar et al., 1997, Synapse, 26(3), 199; Wu-pong et al., 1999, BioPharm, 12(1), 32; Bannai et al., 1998, Brain Res. Protoc., 3(1), 83; Simantov et al., 1996, Neuroscience, 74(1), 39. Nucleic acid molecules of the invention are therefore amenable to delivery to and uptake by cells that express a target nucleic acid for modulation of gene expression.

The delivery of compounds and compositions of the invention can be accomplished by a variety of different strategies. Traditional approaches to CNS delivery include, but are not limited to, intrathecal and intracerebroventricular administration, implantation of catheters and pumps, direct injection or perfusion at the site of injury or lesion, injection into the brain arterial system, or by chemical or osmotic opening of the blood-brain barrier. Other approaches include the use of various transport and carrier systems, for example, conjugates and biodegradable polymers. Furthermore, gene therapy approaches, for example, as described in Kaplitt et al., U.S. Pat. No. 6,180,613 and Davidson, WO 04/013280, can be used to express nucleic acid molecules in the CNS.

In one embodiment, the nucleic acid molecules or the invention are administered via pulmonary delivery, such as by inhalation of an aerosol or spray dried formulation administered by an inhalation device or nebulizer, providing rapid local uptake of the nucleic acid molecules into relevant pulmonary tissues. Solid particulate compositions containing respirable dry particles of micronized nucleic acid compositions can be prepared by grinding dried or lyophilized nucleic acid compositions, and then passing the micronized composition through, for example, a 400 mesh screen to break up or separate out large agglomerates. A solid particulate composition comprising the nucleic acid compositions of the invention can optionally contain a dispersant which serves to facilitate the formation of an aerosol as well as other therapeutic compounds. A suitable dispersant is lactose, which can be blended with the nucleic acid compound in any suitable ratio, such as a 1 to 1 ratio by weight.

Aerosols of liquid particles comprising a nucleic acid composition of the invention can be produced by any suitable means, such as with a nebulizer (see for example U.S. Pat. No. 4,501,729). Nebulizers are commercially available devices which transform solutions or suspensions of an active ingredient into a therapeutic aerosol mist either by means of acceleration of a compressed gas, typically air or oxygen, through a narrow venturi orifice or by means of ultrasonic agitation. Suitable formulations for use in nebulizers comprise the active ingredient in a liquid carrier in an amount of up to 40% w/w preferably less than 20% w/w of the formulation. The carrier is typically water or a dilute aqueous alcoholic solution, preferably made isotonic with body fluids by the addition of, for example, sodium chloride or other suitable salts. Optional additives include preservatives if the formulation is not prepared sterile, for example, methyl hydroxybenzoate, anti-oxidants, flavorings, volatile oils, buffering agents and emulsifiers and other formulation surfactants.

The aerosols of solid particles comprising the active composition and surfactant can likewise be produced with any solid particulate aerosol generator. Aerosol generators for administering solid particulate therapeutics to a subject produce particles which are respirable, as explained above, and generate a volume of aerosol containing a predetermined metered dose of a therapeutic composition at a rate suitable for human administration. One illustrative type of solid particulate aerosol generator is an insufflator. Suitable formulations for administration by insufflation include finely comminuted powders which can be delivered by means of an insufflator. In the insufflator, the powder, e.g., a metered dose thereof effective to carry out the treatments described herein, is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant. The active ingredient typically comprises from 0.1 to 100 w/w of the formulation.

A second type of illustrative aerosol generator comprises a metered dose inhaler. Metered dose inhalers are pressurized aerosol dispensers, typically containing a suspension or solution formulation of the active ingredient in a liquified propellant. During use these devices discharge the formulation through a valve adapted to deliver a metered volume to produce a fine particle spray containing the active ingredient. Suitable propellants include certain chlorofluorocarbon compounds, for example, dichlorodifluoromethane, trichlorofluoromethane, dichlorotetrafluoroethane and mixtures thereof. The formulation can additionally contain one or more co-solvents, for example, ethanol, emulsifiers and other formulation surfactants, such as oleic acid or sorbitan trioleate, anti-oxidants and suitable flavoring agents. Other methods for pulmonary delivery are known and described in, for example, U.S. patent application No. 20040037780, and U.S. Pat. Nos. 6,592,904; 6,582,728; 6,565,885.

In one embodiment, a compound or composition of the invention is complexed with membrane disruptive agents such as those described in U.S. patent application Publication No. 20010007666, incorporated by reference herein in its entirety including the drawings. In another embodiment, the membrane disruptive agent or agents and the siNA molecule are also complexed with a cationic lipid or helper lipid molecule, such as those lipids described in U.S. Pat. No. 6,235,310, incorporated by reference herein in its entirety including the drawings.

Thus, the invention features a pharmaceutical composition comprising one or more compounds or compositions of the invention in an acceptable carrier, such as a stabilizer, buffer, and the like. The compounds and compositions of the invention can be administered (e.g., RNA, DNA or protein) and introduced into a subject by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration, suppositories for rectal administration, sterile solutions, suspensions for injectable administration, and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or subject, including for example, a human. Suitable forms, in part, depend upon the use or the route of entry, for example, oral, transdermal, or by injection. Such forms should not prevent the composition or formulation from reaching a target cell (i.e., a cell to which the negatively charged nucleic acid is desirable for delivery). For example, pharmacological compositions injected into the blood stream should be soluble. Other factors are known in the art, and include considerations such as toxicity and forms that prevent the composition or formulation from exerting its effect.

By “systemic administration” is meant in vivo systemic absorption or accumulation of drugs in the blood stream followed by distribution throughout the entire body. Administration routes that lead to systemic absorption include, without limitation: intravenous, subcutaneous, intraperitoneal, inhalation, oral, intrapulmonary and intramuscular. Each of these administration routes exposes the siNA molecules of the invention to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cells producing excess repeat expansion genes.

By “pharmaceutically acceptable formulation” is meant a composition or formulation that allows for the effective distribution of the nucleic acid molecules of the instant invention in the physical location most suitable for their desired activity. Non-limiting examples of agents suitable for formulation with the nucleic acid molecules of the instant invention include: P-glycoprotein inhibitors (such as Pluronic P85),; biodegradable polymers, such as poly (DL-lactide-coglycolide) microspheres for sustained release delivery (Emerich, D F et al, 1999, Cell Transplant, 8, 47-58); and loaded nanoparticles, such as those made of polybutylcyanoacrylate. Other non-limiting examples of delivery strategies for the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058.

The invention also features the use of the composition comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

The present invention also includes compositions prepared for storage or administration that include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985), hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray, or rectally in dosage unit formulations containing conventional non-toxic pharmaceutically acceptable carriers, adjuvants and/or vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known in the art for the manufacture of pharmaceutical compositions. Such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in a mixture with excipients suitable for the manufacture of aqueous suspensions. Such excipients are suspending agents, for example, sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example, glycerol, propylene glycol, sorbitol, glucose or sucrose. Such formulations can also contain a demulcent, a preservative, and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono-or diglycerides. In addition, fatty acids such as oleic acid find use in the preparation of injectables.

The nucleic acid molecules of the invention can also be administered in the form of suppositories, e.g., for vaginal or rectal administration of the drug. These compositions can be prepared by mixing the drug with a suitable non-irritating excipient that is solid at ordinary temperatures but liquid at body temperature and will therefore melt in the body to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per subject per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular subject depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

In one embodiment, the invention comprises compositions suitable for administering compounds and compositions of the invention to specific cell types. For example, the asialoglycoprotein receptor (ASGPr) (Wu and Wu, 1987, J. Biol. Chem. 262, 4429-4432) is unique to hepatocytes and binds branched galactose-terminal glycoproteins, such as asialoorosomucoid (ASOR). In another example, the folate receptor is overexpressed in many cancer cells. Binding of such glycoproteins, synthetic glycoconjugates, or folates to the receptor takes place with an affinity that strongly depends on the degree of branching of the oligosaccharide chain, for example, triatennary structures are bound with greater affinity than biatenarry or monoatennary chains (Baenziger and Fiete, 1980, Cell, 22, 611-620; Connolly et al., 1982, J. Biol. Chem., 257, 939-945). Lee and Lee, 1987, Glycoconjugate J., 4, 317-328, obtained this high specificity through the use of N-acetyl-D-galactosamine as the carbohydrate moiety, which has higher affinity for the receptor, compared to galactose. This “clustering effect” has also been described for the binding and uptake of mannosyl-terminating glycoproteins or glycoconjugates (Ponpipom et al., 1981, J. Med. Chem., 24, 1388-1395). The use of galactose, galactosamine, or folate based conjugates to transport exogenous compounds across cell membranes can provide a targeted delivery approach to, for example, the treatment of liver disease, cancers of the liver, or other cancers. The use of bioconjugates can also provide a reduction in the required dose of therapeutic compounds required for treatment. Furthermore, therapeutic bioavialability, pharmacodynamics, and pharmacokinetic parameters can be modulated through the use of nucleic acid bioconjugates of the invention. Non-limiting examples of such bioconjugates are described in Vargeese et al., U.S. Ser. No. 10/201,394, filed Aug. 13, 2001; and Matulic-Adamic et al., U.S. Ser. No. 10/151,116, filed May 17, 2002.

In one embodiment, nucleic acid molecules of the invention are complexed with or covalently attached to nanoparticles, such as Hepatitis B virus S, M, or L evelope proteins (see for example Yamado et al., 2003, Nature Biotechnology, 21, 885). In one embodiment, nucleic acid molecules of the invention are delivered with specificity for human tumor cells, specifically non-apoptotic human tumor cells including for example T-cells, hepatocytes, breast carcinoma cells, ovarian carcinoma cells, melanoma cells, intestinal epithelial cells, prostate cells, testicular cells, non-small cell lung cancers, small cell lung cancers, etc.

The compounds of the present invention can also be administered to a patient or subject in combination with other therapeutic compounds to increase the overall therapeutic effect. The use of multiple compounds to treat an indication can increase the beneficial effects while reducing the presence of side effects.

Synthesis of Nucleic Acid Molecules

Synthesis of nucleic acids greater than 100 nucleotides in length is difficult using automated methods, and the cost of such molecules is prohibitive. In this invention, small nucleic acid motifs (“small” refers to nucleic acid motifs less than about 100 nucleotides in length, preferably less than about 80 nucleotides in length, and more preferably less than about 50 nucleotides in length; e.g., antisense oligonucleotides, hammerhead or NCH ribozymes) are preferably used for exogenous delivery. The simple structure of these molecules increases the ability of the nucleic acid to invade targeted regions of RNA structure. Exemplary molecules of the instant invention are chemically synthesized, and others can similarly be synthesized.

Oligonucleotides (for example DNA) are synthesized using protocols known in the art as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on standard equipment (e.g., a 394 Applied Biosystems, Inc. synthesizer) using a 0.2 μmol scale protocol with a 2.5 min coupling step for 2′-O-methylated nucleotides and a 45 sec coupling step for 2′-deoxy nucleotides. Table 1 outlines the amounts and the contact times of the reagents used in the synthesis cycle.

TABLE 1

| | | | | | |
|---|---|---|---|---|---|
| Reagent | Equivalents | Amount | Wait Time* DNA | Wait Time* 2′-O-methyl | Wait Time* RNA |

A. 2.5 μmol Synthesis Cycle ABI 394 Instrument

| | | | | | | |
|---|---|---|---|---|---|---|
| Phosphoramidites | 6.5 | 163 | μL | 45 sec | 2.5 min | 7.5 min |
| S-Ethyl Tetrazole | 23.8 | 238 | μL | 45 sec | 2.5 min | 7.5 min |
| Acetic Anhydride | 100 | 233 | μL | 5 sec | 5 sec | 5 sec |
| N-Methyl | 186 | 233 | μL | 5 sec | 5 sec | 5 sec |
| Imidazole | | | | | | |
| TCA | 176 | 2.3 | mL | 21 sec | 21 sec | 21 sec |
| Iodine | 11.2 | 1.7 | mL | 45 sec | 45 sec | 45 sec |
| Beaucage | 12.9 | 645 | μL | 100 sec | 300 sec | 300 sec |
| Acetonitrile | NA | 6.67 | mL | NA | NA | NA |

B. 0.2 μmol Synthesis Cycle ABI 394 Instrument

| | | | | | | |
|---|---|---|---|---|---|---|
| Phosphoramidites | 15 | 31 | μL | 45 sec | 233 sec | 465 sec |
| S-Ethyl Tetrazole | 38.7 | 31 | μL | 45 sec | 233 min | 465 sec |
| Acetic Anhydride | 655 | 124 | μL | 5 sec | 5 sec | 5 sec |
| N-Methyl | 1245 | 124 | μL | 5 sec | 5 sec | 5 sec |
| Imidazole | | | | | | |
| TCA | 700 | 732 | μL | 10 sec | 10 sec | 10 sec |
| Iodine | 20.6 | 244 | μL | 15 sec | 15 sec | 15 sec |
| Beaucage | 7.7 | 232 | μL | 100 sec | 300 sec | 300 sec |
| Acetonitrile | NA | 2.64 | mL | NA | NA | NA |

C. 0.2 μmol Synthesis Cycle 96 well Instrument

| | Equivalents: DNA/ | Amount: DNA/2′-O- | | | Wait Time* 2′-O- | Wait Time* |
|---|---|---|---|---|---|---|
| Reagent | 2′-O-methyl/Ribo | methyl/Ribo | | Wait Time* DNA | methyl | Ribo |
| Phosphoramidites | 22/33/66 | 40/60/120 | μL | 60 sec | 180 sec | 360 sec |
| S-Ethyl Tetrazole | 70/105/210 | 40/60/120 | μL | 60 sec | 180 min | 360 sec |
| Acetic Anhydride | 265/265/265 | 50/50/50 | μL | 10 sec | 10 sec | 10 sec |
| N-Methyl | 502/502/502 | 50/50/50 | μL | 10 sec | 10 sec | 10 sec |
| Imidazole | | | | | | |
| TCA | 238/475/475 | 250/500/500 | μL | 15 sec | 15 sec | 15 sec |
| Iodine | 6.8/6.8/6.8 | 80/80/80 | μL | 30 sec | 30 sec | 30 sec |
| Beaucage | 34/51/51 | 80/120/120 | | 100 sec | 200 sec | 200 sec |
| Acetonitrile | NA | 1150/1150/1150 | μL | NA | NA | NA |

*Wait time does not include contact time during delivery.

Alternatively, syntheses at the 0.2 μmol scale can be performed on a 96-well plate synthesizer, such as the instrument produced by Protogene (Palo Alto, Calif.) with minimal modification to the cycle. In a non-limiting example, a 33-fold excess (60 μL of 0.11 M=6.6 μmol) of 2′-O-methyl phosphoramidite and a 105-fold excess of S-ethyl tetrazole (60 μL of 0.25 M=15 μmol) can be used in each coupling cycle of 2′-O-methyl residues relative to polymer-bound 5′-hydroxyl. In a non-limiting example, a 22-fold excess (40 μL of 0.11 M=4.4 μmol) of deoxy phosphoramidite and a 70-fold excess of S-ethyl tetrazole (40 μL of 0.25 M=10 μmol) can be used in each coupling cycle of deoxy residues relative to polymer-bound 5′-hydroxyl. Average coupling yields on a 394 Applied Biosystems, Inc. synthesizer, determined by colorimetric quantitation of the trityl fractions, are typically 97.5-99%. Other oligonucleotide synthesis reagents for a 394 Applied Biosystems, Inc. synthesizer include, but are not limited to: detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM I₂, 49 mM pyridine, 9% water in THF (PERSEPTIVE™). High quality acetonitrile (e.g., Burdick & Jackson Synthesis Grade) can be used directly from the reagent bottle. S-Ethyltetrazole solution (0.25 M in acetonitrile) can be made up from the solid obtained from standard suppliers (e.g., American International Chemical, Inc.). Alternately, for the introduction of phosphorothioate linkages, Beaucage reagent (3H-1,2-Benzodithiol-3-one 1,1-dioxide, 0.05 M in acetonitrile) is used.

Deprotection of the DNA oligonucleotides can be performed as follows: the polymer-bound trityl-on oligoribonucleotide is transferred to a 4 mL glass screw top vial and suspended in a solution of 40% aq. methylarnine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder. Standard drying or lyophilization methods known to those skilled in the art can be used.

The method of synthesis used for normal RNA including certain enzymatic nucleic acid molecules follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on standard equipment (e.g., a 394 Applied Biosystems, Inc. synthesizer) using a 0.2 μmol scale protocol with a 7.5 min coupling step for alkylsilyl protected nucleotides and a 2.5 min coupling step for 2′-O-methylated nucleotides. Table 1 outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on a 394 Applied Biosystems, Inc. synthesizer, determined by colorimetric quantitation of the trityl fractions, are typically 97.5-99%. Other oligonucleotide synthesis reagents for a 394 Applied Biosystems, Inc. synthesizer include: detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I₂, 49 mM pyridine, 9% water in THF (PERSEPTIVE™). High quality acetonitrile (e.g., Burdick & Jackson Synthesis Grade) can be used directly from the reagent bottle. S-Ethyltetrazole solution (0.25 M in acetonitrile) can be made up from the solid obtained from standard suppliers (e.g., American International Chemical, Inc.). Alternately, for the introduction of phosphorothioate linkages, Beaucage reagent (3H-1,2-Benzodithiol-3-one 1,1-dioxide 0.05 M in acetonitrile) can be used.

Deprotection of the RNA can be performed using either a “two-pot” or “one-pot” protocol as follows. For the two-pot protocol, the polymer-bound trityl-on oligoribonucleotide is transferred to a 4 mL glass screw top vial and suspended in a solution of 40% aqueous (aq.) methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA·3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA·3HF (0.1 mL) is added and the vial is heated at 65° C. for 15 min. The sample is cooled at −20° C. and then quenched with 1.5 M NH₄HCO₃.

For purification of the trityl-on oligomers, the quenched NH₄HCO₃ solution can be loaded onto a C-18 containing cartridge that had been prewashed with acetonitrile followed by 50 mM TEAA. After washing the loaded cartridge with water, the RNA can be detritylated with 0.5% TFA for 13 min. The cartridge can then be washed again with water, salt exchanged with 1 M NaCl and washed with water again. The oligonucleotide can then be eluted with 30% acetonitrile.

Inactive hammerhead ribozymes or binding attenuated control ((BAC) oligonucleotides) can be synthesized by substituting a U for G₅ and a U for A₁₄ (numbering from Hertel, K. J., et al., 1992, Nucleic Acids Res., 20, 3252). Similarly, one or more nucleotide substitutions can be introduced in other enzymatic nucleic acid molecules to inactivate the molecule and such molecules can serve as a negative control.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the example described above including, but not limited to, 96 well format, with the ratio of chemicals used in the reaction being adjusted accordingly.

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention can be modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Ribozymes can be purified by gel electrophoresis using methods known in the art or purified by high pressure liquid chromatography (HPLC; See Wincott et al., Supra, the totality of which is hereby incorporated herein by reference) and can be re-suspended in water.

Indications

Particular conditions and disease states contemplated by the instant invention include, but are not limited to proliferative diseases and conditions and/or cancer including breast cancer, cancers of the head and neck, including various lymphomas such as mantle cell lymphoma, non-Hodgkins lymphoma, adenoma, squamous cell carcinoma, laryngeal carcinoma, cancers of the retina, cancers of the esophagus, multiple myeloma, ovarian cancer, uterine cancer, melanoma, colorectal cancer, lung cancer, bladder cancer, prostate cancer, glioblastoma, lung cancer (including non-small cell lung carcinoma), pancreatic cancer, cervical cancer, head and neck cancer, skin cancers, nasopharyngeal carcinoma, liposarcoma, epithelial carcinoma, renal cell carcinoma, gallbladder adeno carcinoma, parotid adenocarcinoma, endometrial sarcoma, and multidrug resistant cancers. Other proliferative diseases and conditions, such as neovascularization associated with tumor angiogenesis, macular degeneration (e.g., wet/dry AMD), corneal neovascularization, diabetic retinopathy, neovascular glaucoma, and myopic degeneration, as well as restenosis and polycystic kidney disease, are contemplated by the invention. In addition, inflammatory diseases and conditions such as inflammation, acute inflammation, chronic inflammation, atherosclerosis, restenosis, asthma, allergic rhinitis, atopic dermatitis, septic shock, rheumatoid arthritis, inflammatory bowl disease, inflammotory pelvic disease, pain, ocular inflammatory disease, celiac disease, deep dermal burn, Leigh Syndrome, Glycerol Kinase Deficiency, Familial eosinophilia (FE), autosomal recessive spastic ataxia, laryngeal inflammatory disease; Tuberculosis, Chronic cholecystitis, Bronchiectasis, Silicosis and other pneumoconioses, as well as autoimmune diseases and conditions such as multiple sclerosis, diabetes mellitus, lupus, celiac disease, Crohn's disease, ulcerative colitis, Guillain-Barre syndrome, scleroderms, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, fibromyalgia, Menier's syndrome, and transplantation rejection (e.g., prevention of allograft rejection) are contemplated. Other diseases or conditions that are related to or will respond to the levels of a target nucleic acid in a cell or tissue, alone or in combination with other therapies, are further contemplated by the invention.

The use of statins, anti-inflammatory compounds, immunomodulations, radiation treatments and chemotherapeutics as are known in the art are non-limiting examples of chemotherapeutic agents that can be combined with or used in conjunction with the nucleic acid molecules (e.g. siNA molecules) of the instant invention. Those skilled in the art will recognize that other compounds and therapies can similarly be readily combined with the nucleic acid molecules of the instant invention (e.g. siNA molecules) and are hence within the scope of the instant invention. Such compounds and therapies are well known in the art and include, without limitation, Gemcytabine, cyclophosphamide, folates, antifolates, pyrimidine analogs, fluoropyrimidines, purine analogs, adenosine analogs, topoisomerase I inhibitors, anthrapyrazoles, retinoids, antibiotics, anthacyclins, platinum analogs, alkylating agents, nitrosoureas, plant derived compounds such as vinca alkaloids, epipodophyllotoxins, tyrosine kinase inhibitors, taxols, radiation therapy, surgery, nutritional supplements, gene therapy, radiotherapy, for example 3D-CRT, immunotoxin therapy, for example ricin, and monoclonal antibodies. Specific examples of chemotherapeutic compounds that can be combined with or used in conjuction with the nucleic acid molecules of the invention include, but are not limited to, Paclitaxel; Docetaxel; Methotrexate; Doxorubin; Edatrexate; Vinorelbine; Tomaxifen; Leucovorin; 5-fluoro uridine (5-FU); Ionotecan; Cisplatin; Carboplatin; Amsacrine; Cytarabine; Bleomycin; Mitomycin C; Dactinomycin; Mithramycin; Hexamethylmelamine; Dacarbazine; L-asperginase; Nitrogen mustard; Melphalan, Chlorambucil; Busulfan; Ifosfamide; 4-hydroperoxycyclophosphamide; Thiotepa; Irinotecan (CAMPTOSAR®, CPT-11, Camptothecin-11, Campto) Tamoxifen; Herceptin; IMC C225; ABX-EGF; and combinations thereof. The above list of compounds are non-limiting examples of compounds and/or methods that can be combined with or used in conjunction with the nucleic acid molecules (e.g. siNA) of the instant invention for oncology and related diseases and disorders. Those skilled in the art will recognize that other drug compounds and therapies can similarly be readily combined with the nucleic acid molecules of the instant invention (e.g., siNA, siRNA, ssRNA, dsRNA, antisense, aptamers, ribozymes, enzymatic nucleic acids, DNAzymes, decoys, triplex-forming oligonucleotides, 2′,5′-oligoadenylate chimeras molecules) for treatment of other diseases and conditions, such as inflammatory, allergic, and autoimmune diseases and conditions, and are hence within the scope of the instant invention.

The molecules of the invention can also be used in conjunction with other known methods, therapies, or drugs. For example, the use of monoclonal antibodies (e.g.; mAb IMC C225, mAb ABX-EGF) treatment, tyrosine kinase inhibitors (TKIs), for example OSI-774 and ZD1839, chemotherapy, and/or radiation therapy, are all non-limiting examples of methods that can be combined with or used in conjunction with the compounds of the instant invention. Common chemotherapies that can be combined with nucleic acid molecules of the present invention include, but are not limited to, various combinations of cytotoxic drugs to kill cancer cells. These drugs include, but are not limited to, paclitaxel (Taxol), docetaxel, cisplatin, methotrexate, cyclophosphamide, doxorubin, fluorouracil carboplatin, edatrexate, gemcitabine, vinorelbine, and the like. Those skilled in the art will recognize that other drug compounds and therapies can be similarly and readily combined with the compounds of the instant invention and are hence within the scope of the instant invention.

Diagnostic Uses

The compounds of the present invention, for example, monoclonal antibodies and/or mAb nucleic acid conjugate molecules, can be used as diagnostic tools to detect the presence of a nucleic acid molecule in a biological system or sample. Specifically, compounds of the invention are used to detect nucleic acid molecules without the need for labeling the nucleic acid molecules of interest. This feature is especially important during clinical trials in which nucleic acid therapeutics need to be assayed to determine the pharmacokinetic and metabolic properties of these compounds. In preclinical development of nucleic acid therapeutics, tagged versions of the nucleic acid therapeutic are typically used to evaluate the in vivo characteristics of a particular compound. However, during clinical trials in humans, such tags cannot be used. The monoclonal antibodies of the invention are therefore useful in bioanalytical applications as diagnostic reagents. These antibodies are used to detect the presence of target nucleic acid molecules in samples derived from a variety of sources, including biological samples derived from a patient or subject in a clinical trial. As such, the antibodies of the invention can be used to quantitate the amount of nucleic acid therapeutic in a sample. Methods of quantitation using antibody systems are known in the art, see for example Johansen et al., U.S. Pat. No. 6,087,188; Ramakrishnan, U.S. Pat. No. 5,395,938; and Fujisawa, et al., U.S. Pat. No. 5,028,524; all incorporated by reference herein.

### EXAMPLE 1

### Development of a CA1USR Antibody Specific for 2′-deoxy-2′-C-allyl Uridine Containing Nucleic Acid Molecules

As the ANGIOZYME® ribozyme therapeutic has moved through preclinical studies and into clinical trials, the need has arisen for a reagent to detect this molecule in blood and tissue samples. For preclinical studies, addition of tags such as 2′ bromo-deoxyuridine (BrdU) and fluorescein to the ribozyme during synthesis is useful for localization studies. In clinical trials, however, the ribozymes administered therapeutically do not carry such tags. For clinical localization studies, a monoclonal antibody (mAb) was developed to recognize the non-native 2′-deoxy-2′-C-allyl Uridine modification that is present at a single site in the ANGIOZYME® ribozyme therapeutic. The data presented herein show that the mAb CAIUSR, has a high degree of affinity for the 2′-deoxy-2′-C-allyl Uridine modification. Nucleotides that do not contain the 2′-deoxy-2′-C-allyl Uridine modification were incapable of competing for the binding of the CALUSR mAb to 2′-deoxy-2′-C-allyl Uridine coupled to protein. Replacement of 2′-deoxy-2′-C-allyl Uridine with any of a series of commonly employed nucleotide modifications drastically reduced mAb binding, which implies that the epitope seen by CA1USR comprises both the C-allyl modification and uridine. Finally, the mAb was used to directly localize two structurally different ribozymes containing the 2′-deoxy-2′-C-allyl Uridine modification in the kidneys of mice treated with the ribozymes, and its utility demonstrated as an in vivo detection reagent.

Conjugation of 2′-deoxy-2′-C-allyl Uridine to Proteins

2′-deoxy-2′-C-allyl Uridine 5′-phosphate was synthesized from 2′-deoxy-2′-C-allyl Uridine (Beigelman et al., 1995, Nucleic Acids Research, 23, 4434) by the method of Yoshikawa (Yoshikawa et al., 1969, Bull. Chem. Japan, 42, 3505) with subsequent purification on DEAE Sephadex using a gradient of TEAB (0-0.4 M). The resulting yield was 80% (31P NMR δ=29.3 ppm). 2′-deoxy-2′-C-allyl Uridine 5′-phosphate was covalently coupled to either bovine serum albumin (BSA) or keyhole lympet hemocyanin (KLH) through the phosphate group of the nucleotide to primary amines of the protein by carbodiimide chemistry with 1-ethyl-3-diisopropylaminocarbodiimide-HCl (EDC) The method used here was modified from the method published by Halloran and Parker (Halloran and Parker, 1966, J. Immunol., 6, 373) as follows: BSA was dissolved in 10 mM Tris-HCl, pH 7.5, to a concentration of 125mg/mI; 2′-C-allyl-Uridine 5′-phosphate at a concentration of 216.67 mg/ml in water was adjusted to pH 7.5 with SN NaOH. A 300 μl volume of 2′-deoxy-2′-C-allyl Uridine was mixed with 200 μl of the BSA solution; 65 μl of EDC at 1 g/ml was added (=65 mg) and the reaction was incubated for 24 h at room temperature in the dark. The solution was dialyzed at room temperature against multiple changes of 10 mM Tris, pH 7.5, until the spectrophotometric profile of a separate “mock” reaction (nucleotide and protein without EDC) was the same as that of the protein alone. For coupling of 2′-deoxy-2′-C-allyl Uridine 5′-phosphate to KLH, the concentrations of nucleotide and protein were reduced to 10 mg and 3.85 mg, respectively; 100mg of EDC was added; the total volume of the reaction was adjusted to 500 μl with lOM Tris, pH 7.5. The remainder of the procedure was identical to that for the BSA coupling.

Monoclonal Antibody Production

Twelve week-old female SJL/Jax mice were immunized in the left hind footpad with 100 μg 2′-deoxy-2′-C-allyl Uridine-conjugated BSA (based on concentration of BSA) emulsified in Complete Freund's Adjuvant (total volume of 25 μl). Subsequent immunizations were administered at the same site with a minimum interval of two weeks. The second immunization consisted of 100 μg of 2′-deoxy-2′-C-allyl Uridine-BSA conjugate in Incomplete Freund's Adjuvant (IFA); the third immunization consisted of 200 μg of unconjugated 2′-deoxy-2′-C-allyl Uridine in IFA. Three days after a fourth immunization with 100 μg 2′-C-allyl U-conjugated BSA in IFA, the popliteal lymph node was removed and fused with 653 myeloma cells. Fusion products were plated in 96 well plates, and supernatants were screened for specific anti-2′-deoxy-2′-C-allyl Uridine activity, as described below. Positive hybrids were cloned in soft agar.

Fusion Screening Assay

N-oxy-succinimide ester derivatized 96 well plates (DNA-Bind; Costar/Corning, Corning, N.Y.) were coated overnight at room temperature with 50 ng/well of 2′-deoxy-2′-C-allyl Uridine-KLH. After two washes with phosphate buffered saline, 0.1% Tween (PBS-T), plates were blocked for 1 hour with PBS plus 1% casein (Pierce, Rockford, Ill.). Hybridoma supernatant was incubated on the plates for 1 hour, after which wells were washed 4 times with PBS-T. Positive wells were detected with peroxidase-conjugated F(ab)′2 Goat anti-mouse IgG (Pel-Freez, Rogers, Ark.), and developed with TMB substrate (Kirkegaard and Perry, 2 component; Gaithersburg, Md.). Plates were read on a Molecular Dynamics plate reader (Molecular Dynamics, Sunnyvale, Calif.) at 450 nm with 595 nm correction.

Competition Enzyme Immunoassay (EIA)

DNA-Bind plates were coated overnight with 50 ng/well of 2′-deoxy-2′-C-allyl Uridine-conjugated KLH. 60 μl volumes of the purified CA1USR mAb, at a concentration of 500 ng/ml, were incubated at 37° C. for 30 minutes with 60 μl volumes of a series of concentrations of competitor nucleotides, from a concentration of 7.8 to 500 μg/ml. The antibody-competitor mixtures were then plated on the 2′-C-allyl-KLH coated DNA-Bind plates, and the EIA proceeded as described for the fusion screening assay.

EIA on Biotinylated C-allyl Oligonucleotides and Ribozymes

Oligonucleotides and ribozymes were synthesized with biotin at one end, then bound to streptavidin plates (Hi-Bind Streptavidin plates; Roche Molecular Systems, Indianapolis, Ind.) for 1 hour at room temperature. CA1USR MAb was added at 1 g/ml, and incubated for 1 hour. The remainder of the EIA proceeded as described in the fusion screening assay.

Immunohistochemistry

Tissues were harvested from mice injected subcutaneously with either saline or a ribozyme from 60 to 120 minutes after injection. Animals were perfused with normal saline, then with a solution of 3% paraformaldehyde, 0.5% gluteraldehyde prior to tissue harvest. The tissue was placed in a 30% sucrose solution at 4° C. overnight, then removed, and frozen in a Gentle Jane (Instrumedics; Hackensack, N.J.) Snap-Freezing System with Cryo-Gel Embedding Medium (Instrumedics). Five micron (μm) sections were cut and placed on slides; the slides were allowed to air dry. Prior to staining, slides were quenched in 3% hydrogen peroxide and 0.5% horse serum in PBS, then blocked with M.O.M. Mouse Ig Blocking Reagent (Vector Labs, Burlingame, Calif.) for 1 hour, washed with PBS, and stained with the CA1USR MAb at 5μg/ml in M.O.M. diluent for 30 minutes. After washing, slides were incubated with M.O.M. Biotinylated Anti-Mouse IgG Reagent for 10 minutes, washed, and incubated with Vectastain Elite ABC Reagent for 5 minutes, then developed in DAB chromogen (Vector Labs). Slides were rinsed, counterstained with hematoxylin (Richard-Allan Scientific, Kalamazoo, Mich.), dehydrated, and cover-slipped. Slides were viewed on a Nikon Microphot FXA microscope.

Results

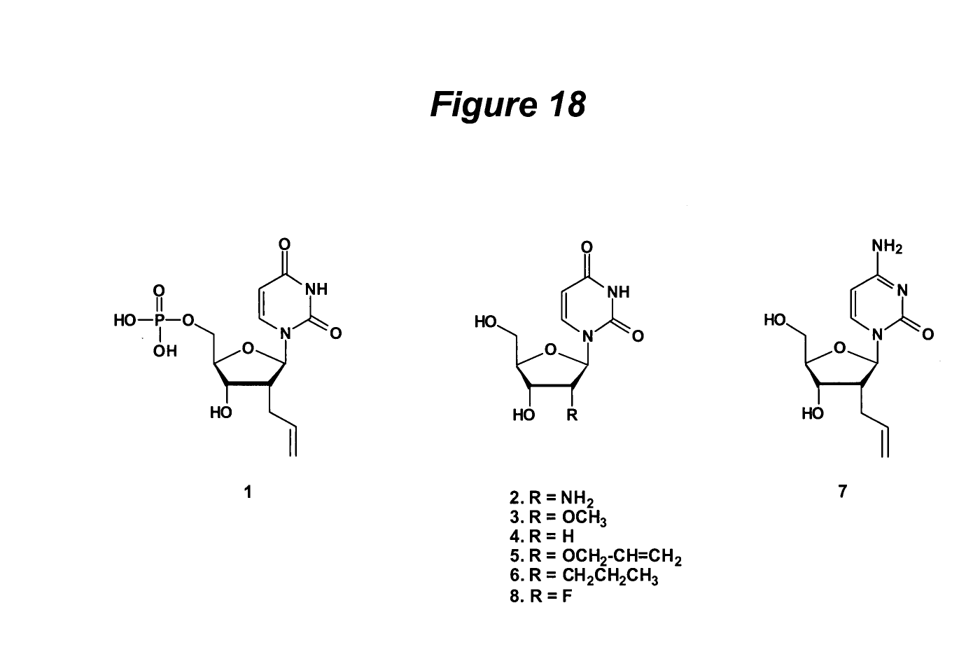

FIG. 18 depicts the chemical structure of the 5′ phosphate of 2′-deoxy-2′-C-allyl Uridine. The molecule was conjugated to either BSA or KLH by EDC. Removal of free from protein-bound 2′-deoxy-2′-C-allyl Uridine phosphate was accomplished by extensive dialysis. Dialysis was considered complete when the OD260/280 was the same for protein alone as that of mock 2′-deoxy-2′-C-allyl Uridine 5′-phosphate-conjugated protein (2′-deoxy-2′-C-allyl Uridine 5′-phosphate and protein without EDC). The degree of substitution on the individual proteins was estimated by the molar concentration of the protein and nucleotide based on the OD280 and extinction coefficient for the protein and OD260 and extinction coefficient for 2′-deoxy-2′-C-allyl Uridine. The degree of substitution for the 2′-deoxy-2′-C-allyl Uridine-BSA conjugate used for immunization is estimated to be five molecules of 2′-deoxy-2′-C-allyl Uridine per molecule of BSA. For KLH, which displays molecular weight heterogeneity, the degree of substitution was estimated to be from 70 to 200 molecules of 2′-deoxy-2′-C-allyl Uridine per molecule of KLH. A hybrid binding to 2′-deoxy-2′-C-allyl Uridine-KLH was identified and cloned. The clone, termed CA1USR, is a murine IgG1k.

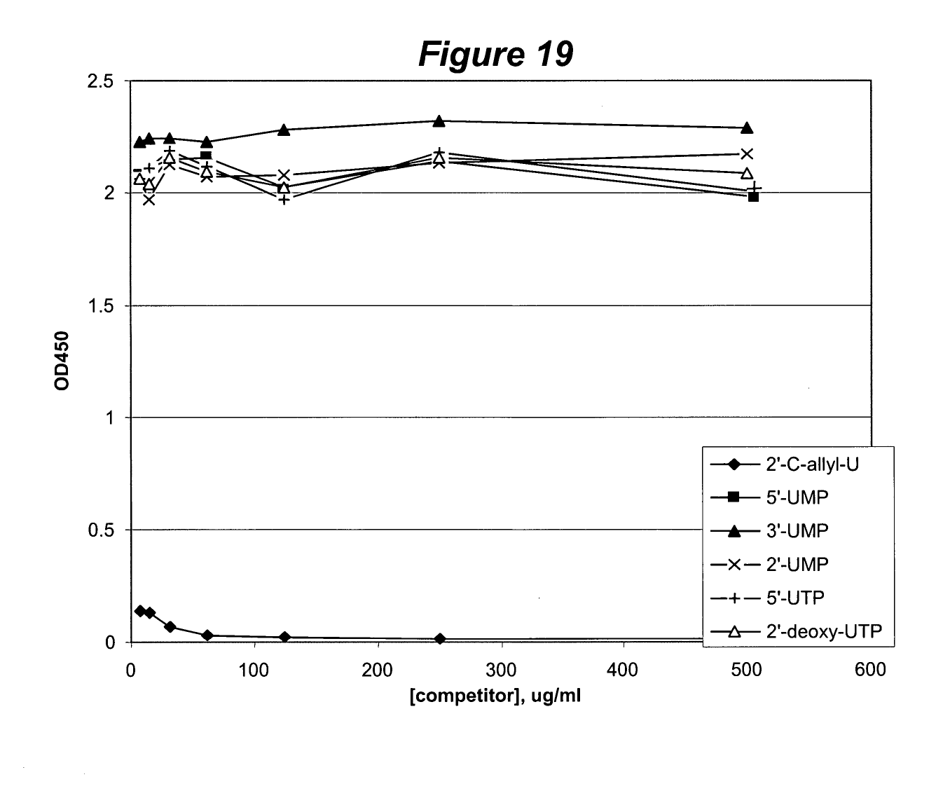

For determination of fine specificity of CA1USR, a series of nucleotides of structure similar to 2′-deoxy-2′-C-allyl Uridine were used to inhibit the binding of the CA1 USR mAb to 2′-deoxy-2′-C-allyl Uridine-coupled KLH captured on DNA-Bind plates. Results are presented in FIG. 19. Of six different uridine nucleotides, only 2′-deoxy-2′-C-allyl Uridine was able to inhibit the binding of CA1USR mAb to 2′-deoxy-2′-C-allyl Uridine-KLH. Other nucleotides containing adenine, cytosine, or guanine had no ability to inhibit 2′-deoxy-2′-C-allyl Uridine mAb binding.

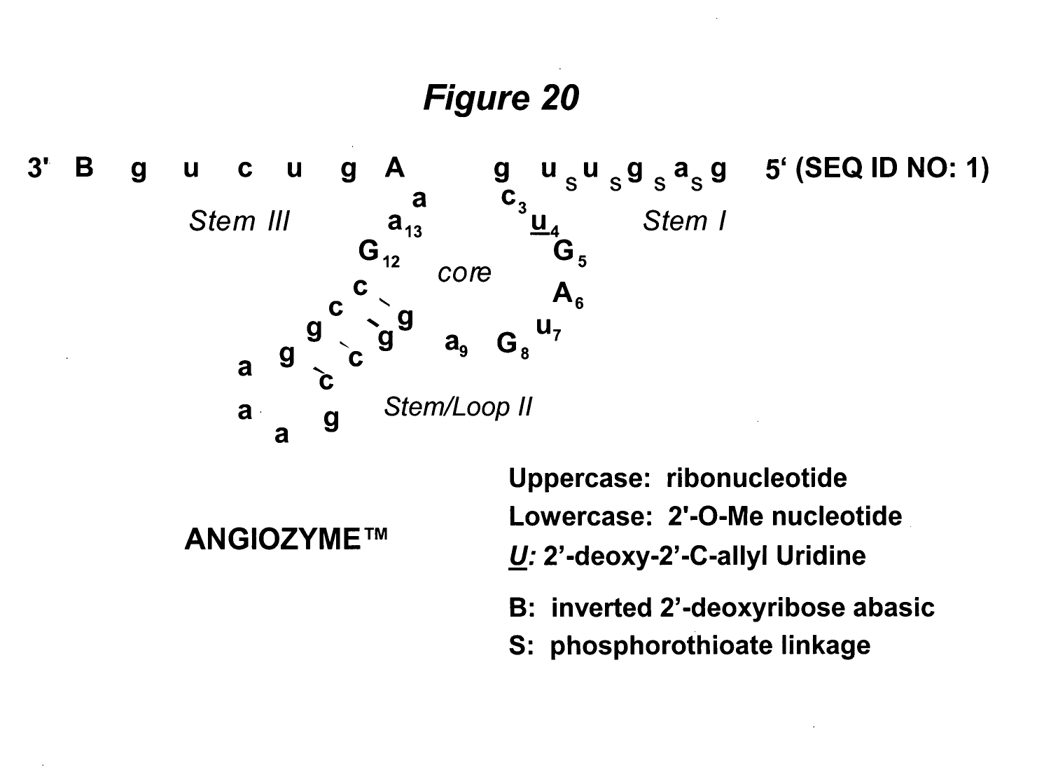

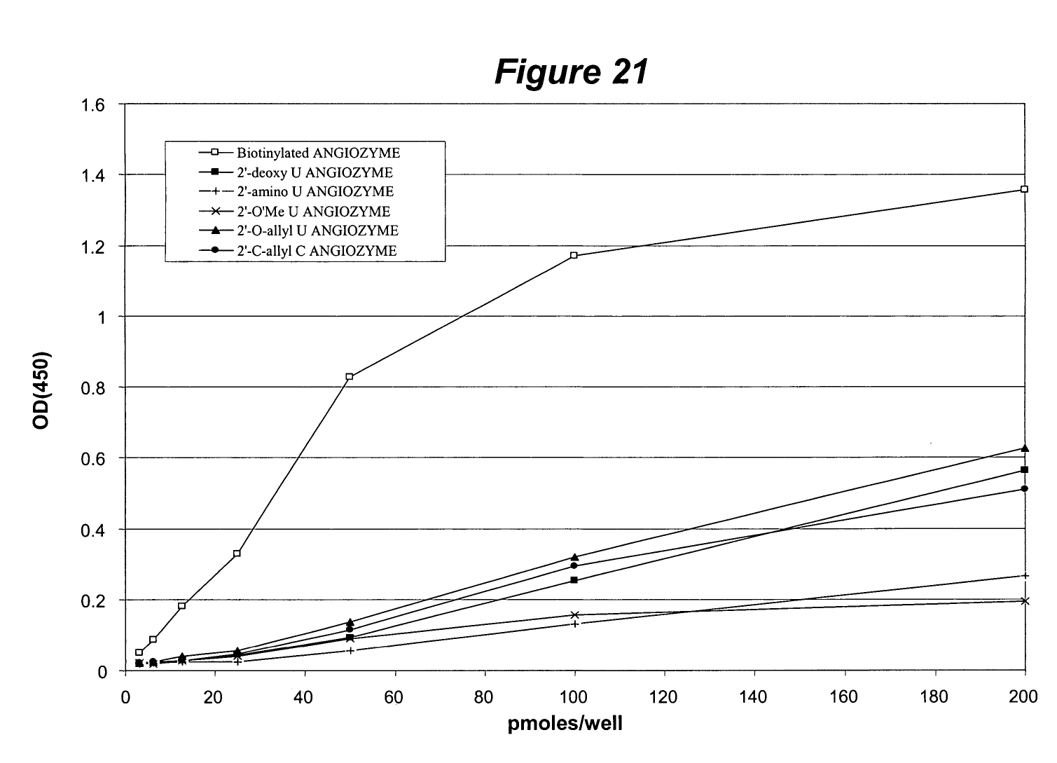

A synthetic ribozyme containing the 2′-deoxy-2′-C-allyl Uridine modification was utilized to determine whether the mAb binds this modification in the context of an oligonucleotide. The structure of the ANGIOZYME® ribozyme directed against VEGFR-1 mRNA is shown in FIG. 20. The ANGIOZYME® ribozyme is a synthetic 35-mer hammerhead ribozyme consisting of VEGFR-1 sequence-specific complementary binding arms, a catalytic core, and a stem-loop that confers the required structure for catalytic activity of the molecule. The 2′-deoxy-2′-C-allyl Uridine modification is shown in the molecule as U4, eight nucleotides from the 5′ end. To further examine which components of 2′-deoxy-2′-C-allyl Uridine are essential for antibody binding, six different U4 2′-ribose modified ribozymes otherwise equivalent in sequence to the ANGIOZYME® ribozyme with biotin at the 5′ end were synthesized: 2′-amino U, 2′-O-methyl Uridine, 2′-deoxy Uridine, 2′-O-allyl Uridine, and 2′-propyl Uridine (FIG. 18). In addition, a ribozyme having the same sequence as the ANGIOZYME® ribozyme but containing 2′-deoxy-2′-C-allyl Cytidine (FIG. 18) instead of 2′-deoxy-2′-C-allyl Uridine was synthesized. The biotinylated, modified ribozymes were added to streptavidin plates at equimolar concentrations to determine the ability of saturating concentrations of the CA1USR mAb to bind each ribozyme. Results are shown in FIG. 21.

The mAb specifically binds the biotinylated ANGIOZYME® ribozyme in a concentration-dependent fashion. mAb binding is drastically reduced on an ANGIOZYME® ribozyme with modified uridines other than 2′-deoxy-2′-C-allyl at the U4 position. Of importance is the lack of binding by the mAb to an ANGIOZYME® ribozyme when 2′-C-allyl uridine is replaced by 2′C-allyl cytidine. The binding and competition experiments suggest that both the 2′-C-allyl modification and the Uracil nucleobase form the epitope that is recognized by the CA1USR mAb.

The CA1USR mAb was utilized to localize the ANGIOZYME® ribozyme in vivo, by immunohistochemistry on tissues from mice treated subcutaneously with either saline or the ANGIOZYME® ribozyme. In kidneys from animals administered three subcutaneous 100 mg/kg injections of the ANGIOZYME® ribozyme, intense 2′-deoxy-2′-C-allyl Uridine mAb immunoreactivity is evident in granular structures within the cytoplasm of cortical tubular epithelial cells. Hematoxylin and eosin microscopy of the ANGIOZYME® ribozyme-treated kidneys showed these granular structures to be basophilic in character. In contrast to the kidneys from the ANGIOZYME® ribozyme treated mice, those from saline treated mice demonstrated no staining with the CA1USR mAb.

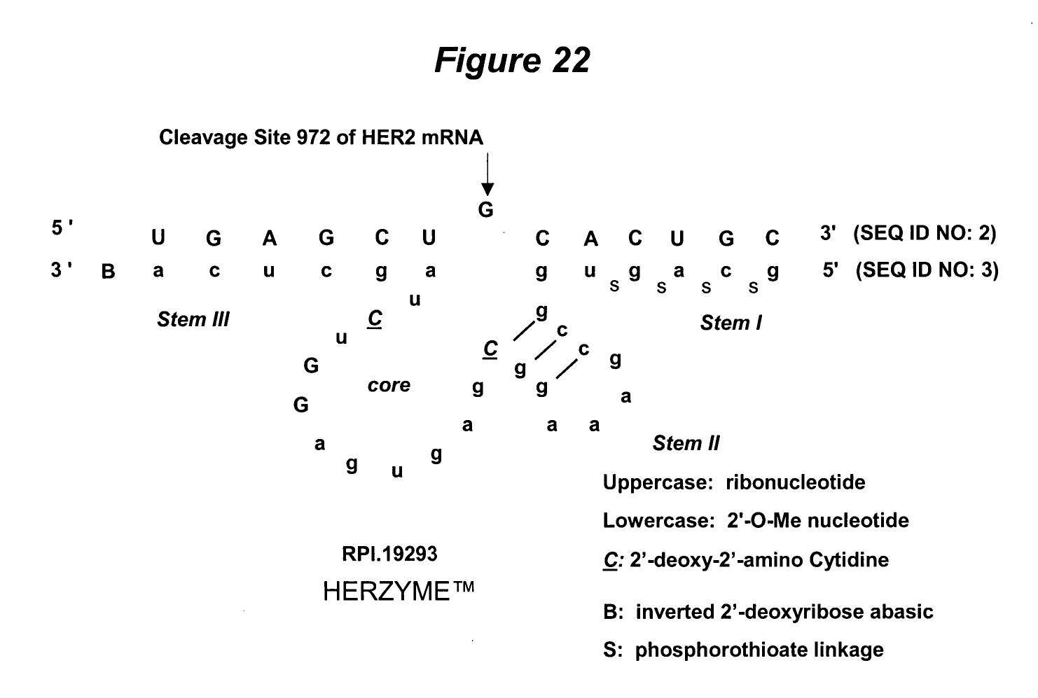

The CA1USR mAb was further examined for its in vivo specificity. A ribozyme targeted to Her2 mRNA with a proposed two-dimensional structure different from the hammerhead motif of the ANGIOZYME® ribozyme was synthesized with the 2′-deoxy-2′-C-allyl modification at the U14 position. The structure of this ribozyme, which is designated by the trademark HERZYME™, is depicted in FIG. 22. Mice were injected subcutaneously three times with a concentration of 100 mg/kg of either the HERZYME™ ribozyme or the 2′-deoxy-2′-C-allyl U14 modified version of the HERZYME™ ribozyme to determine whether the CA1USR mAb could detect the modified ribozyme in vivo. In mice treated with the 2′-C-allyl U14 modified version of the HERZYME™ ribozyme, CA1USR mAb stained granules in kidney cells intensely. In mice treated with the unmodified HERZYME™ ribozyme, there was no staining found in the kidney cells.

As novel therapeutics based on nucleic acid chemistries move into preclinical and clinical studies, the need for reagents and methods of detection both in vitro and in vivo has become paramount to understanding their modes of action, localization, and other important parameters. Monoclonal antibodies provide useful tools for assay development. However, the generation of high affinity mAbs directed against nucleic acid structures has required overcoming immunological barriers. As a general rule, nucleic acids are non-immunogenic; this is probably due to mechanisms of immune tolerance that protect an organism from the pathologic consequences of autoimmune responses. Generation of high affinity mAb's to these structures requires overcoming an animal's natural mechanisms of tolerance. For this reason, applicant utilized the SJL strain of inbred mice, because they have been reported to generate autoantibodies to denatured DNA complexed with methylated BSA better than five other strains of mice, including the most common strain used for monoclonal antibody production, BALB/c (Kearney et al., 1979, J. Immunol., 123, 1548). In fact, during the experiments to generate the 2′-deoxy-2′-C-allyl Uridine mAb described here, applicant immunized three different strains of mice: BALB/c, C57 X BALB F1, and SJL; only SJL mice generated anti-2′-deoxy-2′-C-allyl Uridine mAbs. A second fusion with SJL mice also resulted in generation of a 2′-deoxy-2′-C-allyl Uridine mAb, but of apparent lower affinity than that described here.

Nucleic acids immunized into mice, even with Freund's Complete Adjuvant, behave as haptens, and, even in the SJL mice, resulted in mAb's of the IgM class, which can be less desirable than IgG's. Such small molecules generally require covalent coupling to a carrier protein, such as bovine serum albumin, keyhole limpet hemocyanin, or other carrier protein known in the art, for development of a mature, high affinity immune response. Several methods, including periodate oxidation of the nucleotide for activation and coupling, were used for generating mAbs to other modified nucleosides and nucleotides. mAbs generated by this coupling method include an antibody directed against bromodeoxyuridine and mAbs to methyl methyladenosine and deoxycytodine. Applicant favored the coupling of the phosphate group to primary amines of the carrier proteins through conjugation with EDC, since this conjugation does not affect the structure of either the ribose or base of the coupled nucleotide. This allows the animal to recognize the 2′-deoxy-2′-C-allyl Uridine in a structure more similar to its form in a ribozyme or other nucleic acid molecule.

Based on EIA results, the inferred specificity of the mAb appears to involve both the 2′-deoxy-2′-C-allyl modification and the uracil nucleobase. The inability of any other ribonucleotide to inhibit the binding of the mAb to 2′-deoxy-2′-C-allyl Uridine-coupled to KLH and the fact that the mAb binds very poorly to a ribozyme substituted with 2′-deoxy-2′-C-allyl Cytidine instead of 2′-deoxy-2′-C-allyl Uridine, suggest that both the 2′-deoxy-2′-C-allyl modification and uracil nucleobase are necessary for antibody binding.

Other mAbs have been generated to modified nucleotides, but few appear to have the exquisite specificity of the CA1USR mAb. A series of mAbs to either bromo-deoxy uridine or iodo-deoxy uridine have been generated, among them three mAbs termed IU-1, IU-4, and B-44, respectively. In contrast to the CA1USR mAb, whose epitope does not permit substitution of the 2′-C allyl-U modification, all of these antibodies recognize more than one halogen modification of deoxyuridine. The success of the CA1USR mAb in localizing two structurally different ribozymes containing the 2′-deoxy-2′-C-allyl Uridine modification in mouse kidney proximal tubules confirms its utility for in vivo localization. The results of the ANGIOZYME® ribozyme localization are consistent with those observed in immunohistochemical evaluation of a tetramethylrhodamine labeled form of the ANGIOZYME® ribozyme in murine kidneys. Such findings obviate the necessity for synthesizing different kinds of tagged ribozymes for localization studies in preclinical animal models of disease. Additionally, this mAb enables the localization of 2′-deoxy-2′-C-allyl Uridine-containing ribozymes in biopsies of patients treated with these ribozymes during clinical trials.

Similarly, the development of other monoclonal antibodies to recognize other unique nucleic acid molecules, based on recognition of particular nucleotides or recognition of the particular three dimensional structure of the entire nucleic acid molecule, can be developed using the methods of the instant invention. For example, the use of 2′-deoxy-2′-C-allyl Uridine in place of Uridine in a given nucleic acid molecule of interest can allow for detection of that nucleic acid molecule by CA1USR. Alternately, other monoclonal antibodies can be developed to recognize the unique structure of different nucleic acid molecules, such as enzymatic nucleic acid molecules having SEQ ID NOS: 1, 3, 16, or 17.

### EXAMPLE 2

### In Vivo Localization of Chemically Modified siNA in Murine Tissues by Immunohistochemistry with Novel Monoclonal Antibodies FU1SR and FU2SR

Monoclonal Antibody Production

Immunogens (shown in Table 2) were synthesized with a biotin molecule at the 5′ end of each sequence. Biotinylated immunogen was incubated with avidin (Pierce, Rockford, Ill.) at a 10:1 (biotin-immunogen: avidin) ratio one hour prior to immunization. SJL/Jax female mice (12 weeks of age) were injected with an emulsification of 100 μg immunogen (based on the concentration of avidin) in adjuvant into a footpad. A total of four biweekly immunizations were performed. Cells from the popliteal lymph nodes were fused with the X63Ag8.653 myeloma and plated in 96 well plates. Supernatants from wells containing growing hybrids were screened by enzyme-linked immunoassay against avidin-coated 96 well plates, and against the biotinylated immunogen bound to avidin-coated plates. Hybrids of interest were cloned in soft agarose. The two clones described here (FU1SR and FU2SR) are of the mouse IgG2b subclass.

Specificity Testing Screens

Oligonucleotides were synthesized with biotin at one end, and subsequently bound to avidin-coated 96 well plates. Affinity purified antibodies (1 μg/ml) were added from clones expanded in vitro. After one hour incubation, plates were washed with PBS-0.05 % Tween, and then peroxidase-conjugated goat anti-mouse Ig was added. After washing, reactions were developed with TMB, stopped with 1M phosphoric acid, and read on an ELISA plate reader at 450 nm with 595nm correction.

Immunohistochemistry

Livers from mice injected with either saline, or a cholesterol-conjugated siNA directed against Hepatitis B virus (SEQ ID NOS: 53 and 54) were collected after perfusion with saline followed by a paraformaldehyde-gluteraldehyde solution. After cryosectioning, slides were quenched with 3% hydrogen peroxide. The immunohistochemistry procedure was carried out with the Vector Mouse-On-Mouse kit (Vector Labs, Burlingame, Calif.), according to the manufacturer's protocol. mAbs were used as Protein A purified proteins at a concentration of 5 μg/ml. Digital images were obtained with a Nikon Eclipse 600 microscope equipped with a Nikon Cool-Pix digital camera.

Results

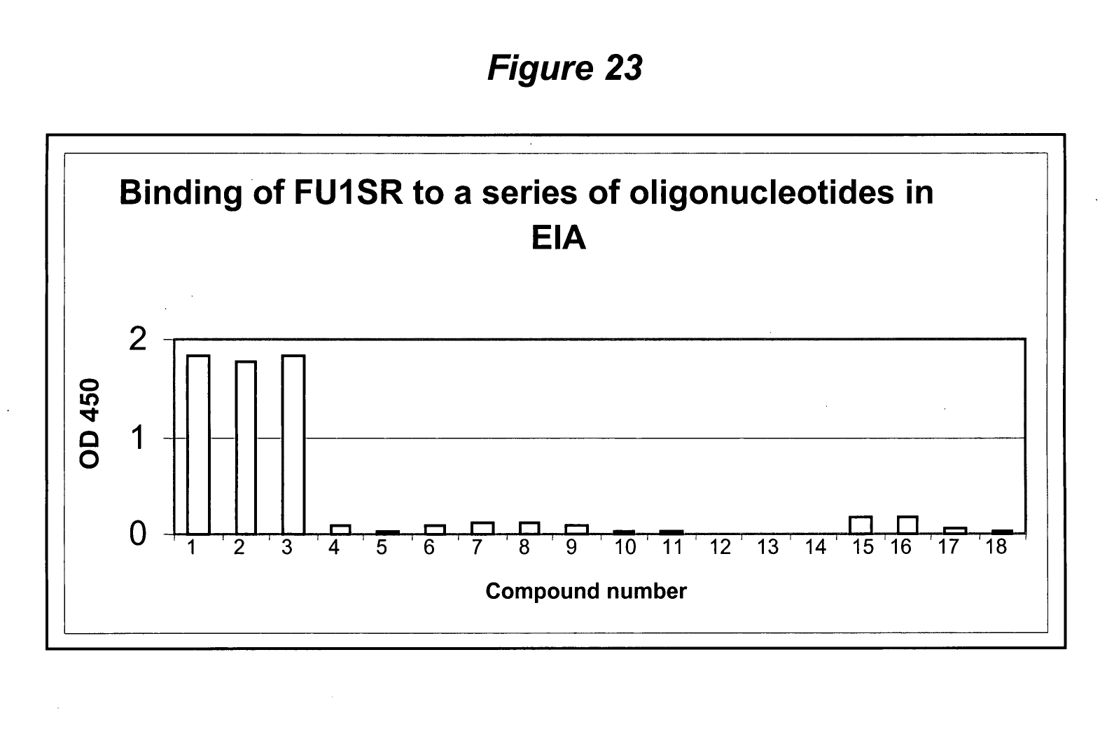

Specificity Binding Data. mAbs FU1SR and FU2SR were derived from immune cells of mice immunized with either or both of the immunogens shown in Table 2. The antibodies were tested against a panel of sequences (Table 3) with different sugar modifications (FIG. 18) to determine binding requirements of the mAbs. FIGS. 23 (FU1SR) and 24 (FJ2SR) depict the binding of two mAbs on a panel of oligonucleotides covering all DNA and RNA bases, and certain 2′ ribose modifications. The mAbs bind only those sequences that contain the 2′-fluorouridine modification. The mAbs fail to bind either compound 15, 16, or 17, which contain the immunizing base without the 2′ fluoro modification, or compound 18, which contains cytidine with a 2′-fluoro modified ribose. The binding data suggests that both the 2′-fluoro modification and the uridine form the epitope(s) bound by the FU1SR and FU2SR mAbs.

Fine Specificity Binding Data. The immunogens for production of these mAbs consist of either a polyA, poly-2′-FU hairpin (FU1SR), or both the hairpin and poly 2′-FU (FU2SR). Applicant designed and tested oligonucleotides containing a single 2′-FU surrounded by different unmodified and modified nucleotides, to test whether the epitope(s) detected by the mAbs were affected by the nature of adjacent nucleotides. Results are shown in Table 4.

Both mAbs bind a single 2′-FU with adjacent polyuridines (compound 19); however, only FU2SR binds a single 2′-FU when only a single uridine is adjacent both 5′ and 3′ (compound 26). Binding of both mAbs is lost when 2′-O-Me modifications are placed both 5′ and 3′ of a single 2′-FU (compounds 21, 21, and 22), including the 2′-O-Me uridine modifications (compound 22). FU2SR binds 2′-FU in the context of adjacent adenosines while FU1SR does not (compound 23). Neither mAb binds 2′-FU with adjacent cytidines or guanosines (compounds 24 and 25). The data suggest that spatial changes that arise adjacent to a single 2′-FU affect binding of the mAb epitopes, and that there are subtle differences in the epitopes bound by FU1SR and FU2SR, respectively.

Oligonucleotides were designed with replacement of a nucleotide either 5′ or 3′ for those oligonucleotides bound by FU2SR, to determine their effects on its epitope (Table 3). Replacement of adenosine with 2′-O-Me adenosine either 5′ or 3′ results in loss of FU2SR binding (compounds 27 and 28). Binding is maintained when 2′-O-Me uridine replaced uridine at the 5′, but not at the 3′-position of the 2′-FU (compounds 29 and 30). Binding is greatly reduced or lost when cytidine replaces uridine, either 5′ or 3′ of 2′-FU (31, 32).

When tested against biotinylated oligonucleotides with lengths and modifications used in siNA molecules, both mAbs bind when poly 2′-FU stretches exist (compound 33), but binding affinities differ between FU1SR and FU2SR when only two 2′-FU's are adjacent (compound 34).

Immunohistochemistry. The ability of both mAbs to detect siNA in vivo is demonstrated by immunohistochemistry. Both mAbs display a signal (brown precipitate of peroxidase substrate) in liver sections from mice injected subcutaneously with a cholesterol conjugated siNA (SEQ ID NOs: 53 and 54, Table 5) duplex directed against HBV four hours after administration, while neither mAb generates a signal on saline-treated liver sections.

One skilled in the art would readily appreciate that the present invention is well adapted to carry out the objects and obtain the ends and advantages mentioned, as well as those inherent therein. The methods and compositions described herein are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that various substitutions and modifications can be made to the invention disclosed herein without departing from the scope and spirit of the invention. Thus, such additional embodiments are within the scope of the present invention and the following claims.

The invention illustratively described herein suitably can be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by various embodiments, optional features, modification and variation of the concepts herein disclosed can be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

TABLE 2

Immunogens used for development of mAbs
directed against 2′-Fluorouridine

| Compound | | SEQ |
|---|---|---|
| Number | Sequence | ID |
| 1 | b AAAAAAAAAA TTCG uuuuuuuuuu B | 18 |
| 2 | b uuuuuuuuuu B | 19 |

Key
b = biotin
A = deoxy Adenosine
T = Thymidine
u = 2′-deoxy-2′-fluoro Uridine
B = inverted abasic

FU1SR was derived from immune cells of a mouse injected four times with biotinylated compound 1-avidin complex

FU2SR was derived from immune cells of a mouse injected three times with biotinylated compound 1 complex, followed by three times with biotinylated compound 2-avidin complex

TABLE 3

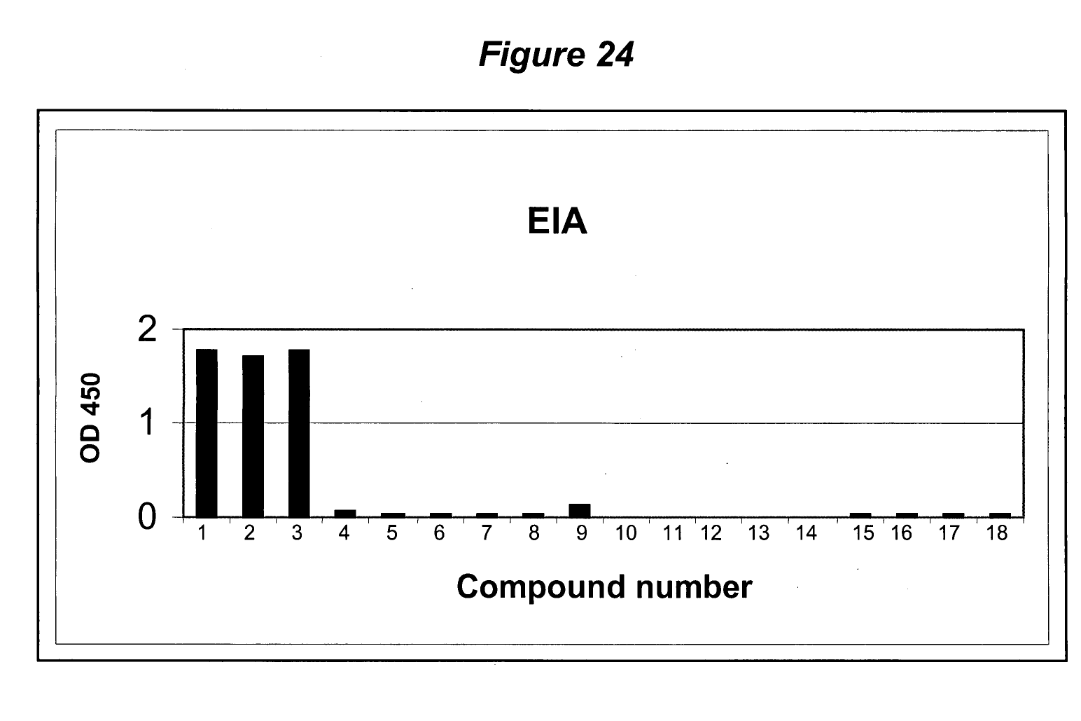

Sequences Used in Selective Binding Studies
Shown in FIGS. 23 and 24

| | | |
|---|---|---|
| Compound | | |
| number | Sequence | SEQ ID |

| | | |
|---|---|---|
| 1 | b AAAAAAAAAA TTCG uuuuuuuuuu B | 18 |
| 2 | b uuuuuuuuuu B | 19 |
| 3 | b aaaaaaaaaa uuccccccg uuuuuuuuuu B | 20 |
| 4 | b AATGTGCACTTCGCTTCACCT B | 21 |
| 5 | b AGAGGTGAAGCGAAGTGCACA B | 22 |
| 6 | b cGaaAgucugB | 23 |
| 7 | b asasasasasa | 24 |
| 8 | b gsgsgsgsgsg | 25 |
| 9 | b gscsasgsuggccg | 26 |
| 10 | b uuuuuu | 27 |
| 11 | b uuuuuu | 28 |
| 12 | b cccccc | 29 |
| 13 | b cscscscscsc | 30 |
| 14 | b AATGTGCACTTC | 31 |
| 15 | b UUUUUUUUUU B | 32 |
| 16 | b AAAAAAAAAATTCGUUUUUUUUUU B | 33 |
| 17 | b UUUUUU | 34 |
| 18 | b cccccCCCCC | 35 |

Sequence Key:
b = biotin
Upper Case Bold = 2′-deoxy
Upper Case = Ribonucleotide
lower case bold = 2′-deoxy-2′-fluoro
Lower case underline = 2′-O-methyl
c = 2′-O-allyl
ₛ = phosphorothioate
B = inverted abasic

TABLE 4

Binding differences between FU1SR and FU2SR:
adjacent nucleotides affect binding

| | | | | |
|---|---|---|---|---|
| Compound | | SEQ | | |
| number | Sequence | ID | FU1USR | FU2SR |

| | | | | |
|---|---|---|---|---|
| 2 | b uuuuuuuu B | 36 | +^{a} | + |
| 19 | b UUUUUuUUUU | 37 | + | + |
| 20 | b agauaaagcg | 38 | − | − |
| 21 | b agcucaagcg | 39 | − | − |
| 22 | b aguuuaagcg | 40 | − | − |
| 23 | b agAuAaagcg | 41 | − | + |
| 24 | b agGuGaagcg | 42 | − | − |
| 25 | b agCuCaagcg | 43 | − | − |
| 26 | b agUuUaagcg | 44 | − | + |
| 27 | b agauAaagcg | 45 | − | − |
| 28 | b agAuaaagcg | 46 | − | − |
| 29 | b aguuUaagcg | 47 | − | + |
| 30 | b agUuuaagcg | 48 | − | − |
| 31 | b agUuCaagcg | 49 | − | − to |
| | | | | +/− |
| 32 | b agCuUaagcg | 50 | − | − |
| 33 | b B GGAcuucucucAAuuuucu | 51 | + | + |
| | TT B | | | |
| 34 | b agaaaauugagagaaguccTₛT | 52 | +/− to | + |
| | | | + | |

^{a}(−) = OD ₄₅₀ < 0.05; (− to +/−) = OD₄₅₀ from 0.05 to 0.5; (+/− to +) = OD₄₅₀ from 0.5 to 1.0; (+) = OD₄₅₀ > 1.5
Sequence Key:
b = biotin
Upper Case Bold = 2′-deoxy
Upper Case = Ribonucleotide
lower case bold = 2′-deoxy-2′-fluoro
lower case underline = 2′-O-methyl
ₛ = phosphorothioate
B = inverted abasic

TABLE 5
Cholesterol Modified siNA targeting HBV RNA

| | |
|---|---|
| 5′ (H)2 ZTa B uGuGcAcuucGcuucAccuTT B | (SEQ ID NO: 53, sense strand) |
| 5′ AGGuGAAGcGAAGuGcAcATsT | (SEQ ID NO: 54, antisense strand) |

1. An isolated antibody having binding affinity for a nucleic acid molecule having a 2′-deoxy-2′-fluoro Uridine nucleoside and/or nucleotide.

2. The isolated antibody of claim 1, wherein said nucleic acid molecule comprises a short interfering nucleic acid (siNA).

3. The isolated antibody of claim 2, wherein said siNA is a duplex siNA.

4. The isolated antibody of claim 3, wherein the 2′-deoxy-2′-fluoro Uridine nucleoside is present in one or both strands of said duplex siNA.

5. The isolated antibody of claim 2, wherein said siNA is a hairpin siNA.

6. The antibody of claim 1, wherein said antibody is a monoclonal antibody.

7. The antibody of claim 1, wherein said antibody is a murine IgG2b antibody.

8. A method for generating a monoclonal antibody (mAb) having binding affinity for nucleic acid molecules having a 2′-deoxy-2′-fluoro Uridine nucleoside or nucleotide comprising:
(a) conjugating a polynucleotide comprising a 2′-deoxy-2′-fluoro Uridine to a carrier protein to form a polynucleotide-protein conjugate;
(b) immunizing a mammal with the conjugate from (a);
(c) obtaining antibody producing cells from the immunized mammal of (b);
(d) fusing the cells obtained in step (c) with a myeloma cell under conditions suitable for generating a hybridoma; and
(e) using supernatant from the hybridoma of (d) in a fusion screen under conditions suitable for isolating the monoclonal antibody.

9. The method of claim 8, wherein the mammal is a mouse.

10. The method of claim 9, wherein said mouse is a SJL mouse.

11. The method of claim 8, wherein the 2′-deoxy-2′-fluoro Uridine polynucleotide is a biotinylated polynucleotide.

12. The method of claim 8, wherein said nucleic acid molecule having a 2′-deoxy-2′-fluoro Uridine nucleoside or nucleotide is a siNA molecule.

13. The method of claim 12, wherein said siNA is a duplex siNA.

14. The method of claim 12, wherein said siNA is a hairpin siNA.

15. A method for generating a monoclonal antibody (mAb) having binding affinity for a short interfering nucleic acid (siNA) comprising:
(a) conjugating the siNA to a carrier protein to form a siNA-protein conjugate;
(b) immunizing a mammal with the conjugate from (a);
(c) obtaining antibody producing cells from the immunized mammal of (b);
(d) fusing the cells obtained in step (c) with a myeloma under conditions suitable for generating a hybridoma; and
(e) using supernatant from the hybridoma of (d) in a fusion screen under conditions suitable for isolating the monoclonal antibody.

16. The method of claim 15, wherein said siNA is a duplex siNA.

17. The method of claim 15, wherein said siNA is a hairpin siNA.

18. A method for detecting the presence of a nucleic acid molecule having a 2′-deoxy-2′-fluoro Uridine nucleotide in a patient comprising:
(a) obtaining a biological sample from the patient; and
(b) contacting the sample of (a) with a monoclonal antibody having specific binding affinity for the nucleic acid molecule having a 2′-deoxy-2′-fluoro Uridine nucleotide under conditions suitable for detecting the presence of said nucleic acid molecule in the patient.

19. The method of claim 18, wherein said nucleic acid molecule having a 2′-deoxy-2′-fluoro Uridine nucleotide is a siNA molecule.

20. The method of claim 19, wherein said siNA is a duplex siNA.

21. The method of claim 19, wherein said siNA is a hairpin siNA.

22. A method for detecting the presence of a siNA in a patient comprising:
(a) obtaining a biological sample from the patient; and
(b) contacting the sample of (a) with a monoclonal antibody having specific binding affinity for the siNA under conditions suitable for detecting the presence of said siNA in the patient.

23. The method of claim 22, wherein the patient is a patient treated with a siNA molecule having one or more 2′-deoxy-2′-fluoro Uridine nucleotides.

24. A method for screening candidate 2′-deoxy-2′-fluoro modified siNA molecules for bioavailability in a mammal comprising:
(a) administering the candidate siNA molecule to the mammal;
(b) obtaining a biological sample from the mammal; and
(c) contacting the sample of (b) with the antibody of claim 1 under conditions suitable for detecting the presence of the siNA molecule in the sample.

25. The method of claim 24, wherein the mammal is a mouse, rat, or pig.

26. The method of claim 24, wherein the mammal is a human.

27. A method for determining the level of a 2′-deoxy-2′-fluoro modified siNA in a mammal comprising:
(a) administering the candidate siNA molecule to the mammal;
(b) obtaining a biological sample from the mammal;
(c) contacting the sample of (b) with the antibody of claim 1 under conditions suitable for detecting the presence of the siNA molecule in the sample, and
(d) assaying for the siNA molecule in the sample under conditions suitable to determine the level of the siNA molecule in the sample and/or mammal.

28. The method of claim 27, wherein the mammal is a mouse, rat, or pig.

29. The method of claim 27, wherein the mammal is a human.
